(19) United States
(12) Patent Application Publication
(10) Pub. No.: US 2023/0304075 A1
(43) Pub. Date: Sep. 28, 2023

(54) NUCLEIC ACID-BASED BIOSENSOR AND ASSOCIATED METHODS

(71) Applicant: FACible BioDiagnostics LLC, Meridian, ID (US)

(72) Inventors: Steven J. Burden, Meridian, ID (US); Clémentine FN Gibard Bohachek, Boise, ID (US); Jonathon C. Reeck, Boise, ID (US); Hunter J. Covert, Boise, ID (US); Tanner B. Pollock, Boise, ID (US); Nicholas H. Shults, Boise, ID (US)

(21) Appl. No.: 17/996,928

(22) PCT Filed: Apr. 22, 2021
(86) PCT No.: PCT/US2021/028699

§ 371 Date: Oct. 22, 2022

Related U.S. Application Data
(60) Provisional application No. 63/064,856, filed on Aug. 12, 2020.
(60) Provisional application No. 63/013,999, filed on Apr. 22, 2020.

(51) Int. Cl. C12Q 1/6825; C12Q 1/70

(52) U.S. Cl. CPC C12Q 1/6825; C12Q 1/701

(57) ABSTRACT

The present disclosure relates to biosensors comprising a sensor region, a linker region, and a reporter region. The sensor region is an aptamer and includes a target domain configured to bind to a target and a reporter domain configured to bind to a reporter. The linker domain operably connects the target domain to the reporter domain. Binding of the target to the target domain results in a conformational change, such as an allosteric change, to the aptamer resulting in a second signal emitted by the reporter that differs from a first signal emitted by the reporter compared to the target unbound state. Methods of selecting biosensors and their use to detect the presence of a target in a sample are provided herein.

### CROSS-REFERENCE TO RELATED APPLICATION(S)

This application claims the benefit of U.S. Provisional Pat. Application Nos. 63/013,999, filed Apr. 22, 2020, and 63/064,856, filed Aug. 12, 2020, both of which are incorporated herein by reference in their entirety.

### TECHNICAL FIELD

The present application relates generally to nucleic acid-based biosensors comprising a reporter (e.g., an RNA aptamer), a linker, and a reporter, and to methods of preparing and using the same. The present disclosure relates to the fields of biology, chemistry, medicinal chemistry, medicine, molecular biology, and pharmacology.

### BACKGROUND

Ribonucleic acid (RNA) aptamers (RNA aptamers and aptamers) are short, single-stranded RNA molecules that fold into stable three-dimensional shapes and are useful for binding to certain structural features of target molecules. RNA aptamers having high affinity and specificity for target molecules, such as proteins, nucleic acids, small molecules, and ions, have previously been selected from complex libraries using the Selective Enrichment of Ligands by Exponential Enrichment (SELEX) protocol (Tuerk and Gold, 1990; Ellington and Szostak, 1992, Ellington et al., 1990, In vitro selection of RNA molecules that bind specific ligands. Nature 346: 818-822. https://doi.org/10.1038/346818a0; Bock et al. 1992, Selection of single-stranded DNA molecules that bind and inhibit human thrombin. Nature 355: 564-566. https://doi.org/10.1038/355564a0, both herein incorporated in their entirety). Most therapeutic RNA aptamers are exogenously administered to cells that express a target molecule (e.g., a target cell) by binding to extracellular domains of certain cell surface proteins. These have been used to inhibit a function of the target molecule or as vehicles to deliver a therapeutic agent to the target cell. (Zhou, et al., 2009; Famalingam, et al., 2011; Ditzler, et al., 2011; Whatley, et al., 2013; Shum, Zhou and Rossi, 2013; Duclair, et al.).

In addition to therapeutic uses, aptamers can be designed to bind a reporter molecule, such as a fluorescent molecule, making them useful reagents (e.g., biosensors) for diagnostic and testing purposes. RNA aptamer biosensors have a high fluorescence signal above a certain background level following a conformational change in the aptamer that occurs after target binding in an allosteric site resulting in fluorescence enhancement. RNA Mango aptamers bind biotinylated derivatives of Thiazole Orange with low nanomolar Kd and fluorescence enhancement of approximately 1,100-fold. There are currently several variants of the RNA Mango aptamer, each having with different nucleotide sequences resulting in different ligand affinities and fluorescence enhancements (Dolgosheina et al., 2014, RNA Mango aptamer-fluorophore: a bright, high affinity, complex for RNA labeling and tracking. ACS Chem Biol. http://dx.doi.org/10.1021/cb500499x; Trachman et al., 2019, Structural basis for high-affinity fluorophore binding and activation by RNA Mango. Nat Chem Biol 13: 807-813; Autour et al., 2018, Fluorogenic RNA Mango aptamers for imaging small non-coding RNAs in mammalian cells. Nat Commun 9: 656, all herein incorporated in their entirety by reference). The structure of the original RNA Mango aptamer, Mango-I, includes a single base-paired stem capped by a three-tiered G-quadruplex where the ligand binds. The emitted fluorescent signal can be dim or bright and is detectable above a certain amount of background fluorescence. In certain instances, a DNA aptamer emits a low signal, such as a dim signal, when a target is bound to the binding site and while useful for certain applications, the current structure of RNA Mango-I prevents its use in more complex functionalities. For example, the inclusion of additional nucleotide elements to the G-quadruplex core disrupts ligand binding and/or causes a switching effect.

Other fluorogenic aptamers, such as RNA Spinach, also have G-quadruplex ligand binding sites, but with more than one base-paired stem joined to the quadruple. These additional helical elements have been used in several applications that require more complex structures but lack the high signal to low background ratio of the RNA Mango aptamers making them less sensitive.

Accordingly, there is a need to identify new RNA aptamers useful as biosensors for a variety of different therapeutic and diagnostic purposes, in vitro and in vivo.

### SUMMARY

The application relates generally to biosensors comprising a reporter and an aptamer with at least one stem. The present disclosure provides aptamers of the biosensors having a target domain comprising a randomized region of at least 30 nucleotides that replaces or is disposed within the at least one stem, a reporter domain configured to bind to the reporter, and a linker domain operably connected between the target domain and the reporter domain. In some embodiments, the aptamer is an RNA aptamer. In certain embodiments, the RNA aptamer comprises a nucleotide sequence of GGAACCUN_{Y}AUGAUGGAGAGGCGCAAGGUUAACCGCCUCAGGUUCC (SEQ ID NO: 104), GGAACCCN_{Y}GUGAUGGAGAGGCGCAAGGUUAACCGCCUCAGGUUCC (SEQ ID NO:105), or GGAACCNN_{Y}NUGAUGGAGAGGCGCAAGGUUAACCGCCUCAGGUUCC (SEQ ID N0:106). In such embodiments, the randomized region replaces a first stem and is represented by N_{Y} in the nucleotide sequence. In certain embodiments, N_{Y} is N30, N45, N46, or N60.

In some embodiments, the aptamer is an RNA aptamer. In certain embodiments, the RNA aptamer comprises a nucleotide sequence of N_{Y}CUCGCUUCGGCGAUGAUGGAGAGGCGCAAGGUUAACCGCCUCAGN_{Y} (SEQ ID NO:116), N_{Y}CCCGCUUCGGCGGUGAUGGAGAGGCGCAAGGUUAACCGCCUCAGN_{Y} (SEQ ID NO:117), or N_{Y}CNCGCUUCGGCG NUGAUGGAGAGGCGCAAGGUUAACCGCCUCAGN_{Y} (SEQ ID NO:118). In such embodiments, the randomized region replaces an outside stem and is represented by N_{Y} in the nucleotide sequence. In some embodiments, the randomized region is 30 nucleotides and N_{Y} is N15. In some aspects, the aptamer comprising the randomized region of 30 nucleotides has a nucleotide sequence of NNNNNNNNNNNNNNNCUCGCUUCGGCGAUGAUGGAGAGGCGCAAGGUUAACCGCC UCAGNNNNNNNNNNNNNNN (SEQ ID NO:122) or of NNNNNNNNNNNNNNN CCCGCUUCGGCGGUGAUGGAGAGGCGCAAGGUUAACCGCCUCAGNNNNNNNNNNN NNNN (SEQ ID NO:123), or of NNNNNNNNNNNNNNNCNCGCU UCGGCGNUGAUGGAGAGGCGCAAGGUUAACCGCCUCAGNNNNNNNNNNNNNNN (SEQ ID NO:124).

In some embodiments, the randomized region is 46 nucleotides and N_{Y} is N23. In some aspects, the aptamer comprising the randomized region of 46 nucleotides replaces the outside stem and has a nucleotide sequence of NNNNNNNNNNNNNNNNNNNNNNNCUCGCUUCGGCGAUGAUGGAGAGGCGCAAGGU UAACCGCCUCAGNNNNNNNNNNNNNNNNNNNNNNN (SEQ ID NO:128), NNNNNNNNNNNNNNNNNNNNNNNCCCGCUUCGGCGGUGAUGGAGAGGCGCAAGG UUAACCGCCUCAGNNNNNNNNNNNNNNNNNNNNNNN (SEQ ID NO:129), or of NNNNNNNNNNNNNNNNNNNNNNNCNCGCUUCGGCGNUGAUGGAGAGGCGCAAGGU UAACCGCCUCAGNNNNNNNNNNNNNNNNNNNNNNN (SEQ ID NO:130).

In some embodiments, the randomized region is 60 nucleotides and N_{Y} is N30. In some aspects, the aptamer comprising the randomized region of 60 nucleotides has a nucleotide sequence of NNNNNNNNNNNNNNNNNNNNNNNNNNNNNNCUCGCUUCGGCGAUGAUGGAGAGGC GCAAGGUUAACCGCCUCAGNNNNNNNNNNNNNNNNNNNNNNNNNNNNNN (SEQ ID NO:134), NNNNNNNNNNNNNNNNNNNNNNNNNNNNNNCCCGCUUCGGCGGU GAUGGAGAGGCGCAAGGUUAACCGCCUCAGNNNNNNNNNNNNNNNNNNNNNNNNN NNNNN (SEQ ID NO:135), or of NNNNNNNNNNNNNNN NNNNNNNNNNNNNNNCNCGCUUCGGCGNUGAUGGAGAGGCGCAAGGUUAACCGCC UCAGNNNNNNNNNNNNNNNNNNNNNNNNNNNNNN (SEQ ID NO:136).

In certain embodiments, the RNA aptamer comprises a nucleotide sequence of GGAACCUCGCUUCGGCGAUGAUGGAGN_{Y}CAGGUUCC (SEQ ID NO:119), GGAACCCCGCUUCGGCGGUGAUGGAGN_{Y}CAGGUUCC (SEQ ID NO:120), or GGAACCNCGCUUCGGCGNUGAUGGAGN_{Y}CAGGUUCC (SEQ ID NO:121). In such embodiments, the randomized region replaces an inside stem and is represented by N_{Y} in the nucleotide sequence. In some embodiments, the randomized region is 30 nucleotides and N_{Y} is N30. In some aspects, the aptamer comprising the randomized region of 30 nucleotides has a nucleotide sequence of GGAACCUCGCUUCG GCGAUGAUGGAGNNNNNNNNNNNNNNNNNNNNNNNNNNNNNNCAGGUUCC (SEQ ID NO:125), GGAACCCCGCUUCGGCGGUGAUGGAGNNNNNNNNNNNNNNNNNNNNNN NNNNNNNNCAGGUUCC (SEQ ID NO:126), GGAACCNCGCUUCGGCGNUGA UGGAGNNNNNNNNNNNNNNNNNNNNNNNNNNNNNNCAGGUUCC (SEQ ID NO:127).

In some embodiments, the randomized region is 45 nucleotides and N_{Y} is N45. In some aspects, the aptamer comprising the randomized region of 45 nucleotides has a nucleotide sequence of GGAACCUCGCUUCGGCGAUGAUGGAGNNNNNNN NNNNNNNNNNNNNNNNNNNNNNNNNNNNNNNNNNNNNNCAGGUUCC (SEQ ID NO:131), GGAACCCCGCUUCGGCGGUGAUGGAGNNNNNNNNNNNNNNNNNNNNNN NNNNNNNNNNNNNNNNNNNNNNNCAGGUUCC (SEQ ID NO:132), or GGAACCNCGCUUCGGCGNUGAUGGAGNNNNNNNNNNNNNNNNNNNNNNNNNNNNN NNNNNNNNNNNNNNNNCAGGUUCC (SEQ ID NO:133).

In some embodiments, the randomized region is 60 nucleotides and N_{Y} is N60. In some aspects, the aptamer comprising the randomized region of 60 nucleotides has a nucleotide sequence of GGAACCUCGCUUCGGCGAUGAUGGAGNNNNNNNNNNN NNNNNNNNNNNNNNNNNNNNNNNNNNNNNNNNNNNNNNNNNNNNNNNNNCAGGUU CC (SEQ ID NO:137) or of GGAACCCCGCUUCG GCGGUGAUGGAGNNNNNNNNNNNNNNNNNNNNNNNNNNNNNNNNNNNNNNNNNNN NNNNNNNNNNNNNNNNNCAGGUUCC (SEQ ID NO:138), or GGAACCNCGCUUCGGCGNUGAUGGAGNNNNNNNNNNNNNNNNNNNNNNNNNNNNN NNNNNNNNNNNNNNNNNNNNNNNNNNNNNNNCAGGUUCC (SEQ ID NO:139).

In some embodiments, the nucleotide sequence of the randomized region in the target domain that binds the target is identified by SELEX.

In some embodiments, the reporter is a fluorescent molecule. In certain embodiments, the fluorescent molecule is sulforhodamine-dinitroaniline.

The present disclosure also provides methods of detecting a target in sample comprising contacting the sample with the biosensors of the present technology. In some embodiments, the target is a pathogen, a small molecule, a solvent, or an ion. In certain embodiments, the pathogen is a bacterial pathogen, a viral pathogen, a prokaryotic pathogen, a fungal pathogen, or a combination thereof. In some aspects, the pathogen is adenovirus, coronavirus, human metapneumovirus, human rhinovirus/enterovirus, influenza, parainfluenza, respiratory syncytial virus, bordatella pertussis, chlamydophia penumoniae, SARS-CoV, SARS-CoV2, MERS-CoV, UPEC, E. coli, klebsiella pneumoniae, proteus mirabilis, pseudomonas aeruginosa, staphylococcus saprophyticus, enterococcus faecalis, enterococcus faecim, clostridioides difficile, methicillin-resistant staphylococcus aureus, proteins synthesized by antibiotic resistant bacteria, West Nile virus, Zika virus, Ebola virus, salmonella, equine herpesvirus type I) and type IV, human immunodeficiency virus (HIV), hepatitis A, hepatitis B, hepatitis C, malaria, Dengue virus, norovirus, rotavirus, astrovirus, Marburg virus, rabies, small pox, measles, or hantavirus. In other aspects, the small molecule is a toxin or a pharmaceutical agent. In certain aspects, the small molecule is a cannabinoid, bisphenol A, fluoride, or benzene. In further aspects, the cannabinoid is cannabidiol, cannabinol, or tetrahydrocannabinol. In some aspects, the solvent is acetone, cyclohexane, acetic acid, ethanol, or benzene. In certain aspects, the ion is potassium, chloride, sodium, lithium, magnesium, mercury, or lead.

### BRIEF DESCRIPTION OF THE DRAWINGS

Many aspects of the present disclosure can be better understood with reference to the following figures. The components in the figures are not necessarily to scale. Instead, emphasis is placed on illustrating clearly the principles of the present disclosure.

FIG. 1 is a conceptual illustration of the SRB-2 aptamer backbone sequence, a location of where a randomized region (e.g., N_{Y} position) is disposed on stem 2, and a reporter (sulforhodamine-dinitroaniline, “SR-DN”) having a Kd of 310 nM in accordance with embodiments of the present technology.

FIG. 2 is a conceptual illustration of the SRB-2 aptamer backbone sequence, a location of where a randomized region will be inserted on the outside stem (circle near bottom) or the inside stem (circle near SR-DN), and SR-DN reporter having a Kd of 310 nM in accordance with embodiments of the present technology.

FIGS. 3A and 3B depict predictive thermograms for N30-SRB-2 libraries with primers (FIG. 3A) and without primers (FIG. 3B) in accordance with embodiments of the present technology.

FIGS. 4A and 4B depict predictive thermograms for N45-SRB-2 libraries with primers (FIG. 4A) and without primers (FIG. 4B) in accordance with embodiments of the present technology.

### DETAILED DESCRIPTION

The present technology is directed to biosensors comprising an aptamer (e.g., an aptamer having a target domain, a reporter domain, and a linker domain) and a reporter for determining the presence of a target in a sample, and associated systems and methods of use. Some embodiments of the present technology, for example, are directed to biosensors having aptamers that, upon binding of the target to the target domain, undergo a conformational change resulting in a change in signal emission, reduced or enhanced signal emission, signal quenching, or enhanced signal quenching by the reporter (e.g., a second signal or a second state) compared to a signal emitted when the aptamer is not bound to the target (e.g., a first signal or a first state). The conformational change can be an allosteric change. In some embodiments, the aptamer is based on an SRB-2 aptamer backbone and includes a length of randomized nucleic acids within at least one stem of the SRB-2 aptamer backbone. The length of the randomized region can be selected based on the target of interest and specific aptamer sequences including the randomized region identified using SELEX. In some embodiments, the randomized region is about 15 nucleotides, about 20 nucleotides, about 25 nucleotides, about 30 nucleotides, about 35 nucleotides, about 40 nucleotides, about 45 nucleotides, about 50 nucleotides, about 55 nucleotides, or about 60 nucleotides.

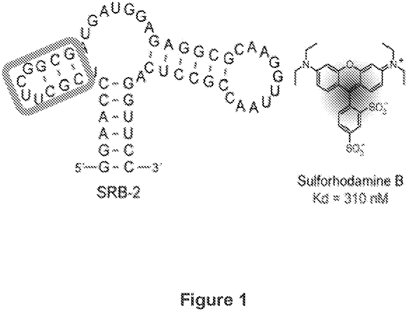

Useful reporters include fluorescent reporters, such as SR-DN. Binding of the aptamer to the target can be detected by a change between a first signal emitted in a target unbound state and a second signal emitted in a target bound state. In some embodiments, the first signal has a greater intensity than the second signal. In other embodiments, the second signal has a greater intensity than the first signal. In either of these embodiments, the signal is determined after quenching has been detected. These aptamers and reporters, and other aptamers and reporters derived from and/or otherwise based upon the aptamers and reporters described herein, are included in embodiments of the present technology. Specific details of several embodiments of the technology are described below with reference to FIGS. 1-4B.

While the present technology is capable of being embodied in various forms, the description below of several embodiments is made with the understanding that the present disclosure is to be considered as an exemplification of the technology and is not intended to limit the technology to the specific embodiments illustrated. Headings are provided for convenience only and are not to be construed to limit the technology in any manner. Embodiments illustrated under any heading may be combined with embodiments illustrated under any other heading.

In the present description, any concentration range, percentage range, ratio range, or integer range is to be understood to include the value of any integer within the recited range and, when appropriate, fractions thereof (such as one tenth and one-hundredth of an integer), unless otherwise indicated. Also, any number range recited herein is to be understood to include any integer within the recited range, unless otherwise indicated. As used herein, the term “about” means ± 20% of the indicated range, value, or structure, unless otherwise indicated. It should be understood that the terms “a” and “an” as used herein refer to “one or more” of the enumerated regions. Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense that one having skill in the art would understand the convention (e.g., “a system having at least one of A, B, and C” would include, but not be limited to, systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense that one having skill in the art would understand the convention (e.g., “a system having at least one of A, B, or C” would include, but not be limited to, systems that have A alone, B alone, C alone, A and B together, A and C together, B and C together, and/or A, B, and C together, etc.). As used herein, the terms “include,” “have,” and “comprise” are used synonymously, which terms and variants thereof are intended to be construed as non-limiting. Further, headings provided herein are for convenience only and do not interpret the scope or meaning of the claimed embodiments.

The present technology has been described in terms of particular embodiments found or proposed by the present inventor to comprise preferred modes for the practice of the technology. It will be appreciated by those of skill in the art that, in light of the present disclosure, numerous modifications and changes can be made in the particular embodiments exemplified without departing from the intended scope of the technology. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

Unless otherwise defined, all technical and scientific terms used herein have the same meaning as commonly understood by one of ordinary skill in the art to which this disclosure pertains. Although methods and materials similar or equivalent to those described herein can be used in the practice of the present disclosure, suitable methods and materials are described below. Unless otherwise indicated, nucleic acids are written left to right in 5′ to 3′ orientation; amino acid sequences are written left to right in amino to carboxy orientation, respectively. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

### Definitions

The term “biosensor”, as used herein, refers to a macromolecule comprising an aptamer and a reporter, and optionally, one or more linkers.

The term “aptamer” refers to any polynucleotide, generally an RNA or a DNA that has a useful biological activity in terms of biochemical activity, molecular recognition or binding attributes to a target. Usually, an aptamer has a molecular activity such as binding to a target at a specific binding site on the target. It is generally accepted that an aptamer, which is specific in its binding to the target, may be synthesized and/or identified by SELEX. Aptamers of the present technology often include two binding sites, a target binding site and a reporter binding site.

The term “systematic evolution of ligands by exponential enrichment” or “SELEX” generally means any method of selecting for an aptamer which binds to a target. SELEX involves screening a pool of random targets for a particular aptamer that binds to a target or has a particular activity that is selectable. Generally, the particular aptamer represents a very small fraction of the target pool, therefore, a round of aptamer amplification, usually via polymerase chain reaction, is employed to increase the representation of potentially useful aptamers. Successive rounds of selection and amplification are employed to exponentially increase the abundance of the particular and useful aptamer. SELEX is described in several publications including, but not limited to, Famulok, M.; Szostak, J. W., In Vitro Selection of Specific Ligand Binding Nucleic Acids, Angew. Chem. 1992, 104, 1001. (Angew. Chem. Int. Ed. Engl. 1992, 31, 979-988.); Famulok, M.; Szostak, J. W., Selection of Functional RNA and DNA Molecules from Randomized Sequences, Nucleic Acids and Molecular Biology, Vol 7, F. Eckstein, D. M. J. Lilley, Eds., Springer Verlag, Berlin, 1993, pp. 271; Klug, S.; Famulok, M., All you wanted to know about SELEX; Mol. Biol. Reports 1994, 20, 97-107; and Burgstaller, P.; Famulok, M. Synthetic ribozymes and the first deoxyribozyme; Angew. Chem. 1995,107, 1303-1306 (Angew. Chem. Int. Ed. Engl. 1995, 34, 1189-1192).

As used herein, the terms “antigen,” “target” and “analyte” are used interchangeably and refer generally to a ligand, small molecule, ion, salt, metal, enzyme, drug, nanoparticle, environmental contaminant, toxin, fatty acid, steroid, hormone, carbohydrate, amino acid, peptide, microbe, virus, nucleic acid, or any other agent which is capable of binding to an aptamer of the present technology. A target is characterized by its ability to be “bound” by the aptamer. Target can also mean the substance used to elicit the production of targeting moieties, such as the production of aptamers through immunizing with the target.

The term “antigen binding site,” “target binding site,” “analyte brining site,” or “epitope” refers to the portion of the target to which the aptamer binds.

The terms “bind,” “binds,” and “specifically binds” refers to the ability of an aptamer to bind to a target with greater affinity than it binds to a non-target. In certain embodiments, specific binding refers to binding for aptamer with an affinity that is at least 10, 50, 100, 250, 500, or 1000 times greater than the affinity for a non-target.

The term “binding affinity” refers to the strength of interaction between an aptamer and its target as a function of its association and dissociation constants. Higher affinities typically mean that the aptamer has a fast on rate (association) and a slow off rate (dissociation). Binding affinities can change under various physiological conditions due to changes that occur to the target or aptamer under those conditions. Binding affinities of the aptamer can also change when a reporter is attached. Binding affinities can also change when slight changes occur to the target, such as changes in the amino acid or nucleotide sequence or glycosylation of the target. Generally, the aptamers of the present disclosure have high binding affinities for their respective targets.

The term “linker” or “linker molecule” refers to any polymer attached to an aptamer or aptamer construct. The attachment may be covalent or non-covalent. It is envisioned that the linker can be a polymer of amino acids or nucleotides. A preferred linker molecule is flexible and does not interfere with the binding of a nucleic acid binding factor to the set of nucleic acid components.

The term “reporter,” as used herein, refers to any substance attachable (e.g., by binding) to an aptamer in which the substance is detectable by a detection method. Non-limiting examples of reporters applicable to this technology include but are not limited to luminescent molecules, chemiluminescent molecules, fluorochromes, fluorescent quenching agents, colored molecules, radioisotopes, mass reporters, biotin, avidin, streptavidin, protein A, protein G, antibodies or fragments thereof, polyhistidine, nickel and its ions, Flag tags, myc tags, heavy metals, enzymes, alkaline phosphatase, peroxidase, luciferase, and colorimetric substrates.

As used herein, the terms “detection method” and “detectable signal” are interchangeable and refer to any method or output of the method known in the art to detect a molecular interaction event, such as binding of an aptamer to a target. Non-limiting examples of detection methods include detecting changes in fluorescence (e.g., FRET, FCCS, decreasing fluorescence, such as fluorescence quenching, or increasing fluorescence, fluorescence polarization), changes in mass, changes in enzymatic activity, and changes chemiluminescence.

The term “effective amount” refers to an amount of an aptamer, either alone or as a part of a composition, such as a biosensor or other composition, that is capable of having any detectable output, such as a detectable signal when combined with a sample, or a therapeutic effect on any symptom, aspect, parameter or characteristics of a disease state or condition when administered to a subject. Such effect need not be absolute to be detectable and/or beneficial.

The terms “recipient,” “individual,” “subject,” “host,” and “patient” are used interchangeably herein and refer to any mammalian subject for whom diagnosis, treatment, or therapy is desired, particularly humans. “Mammal” for purposes of treatment refers to any animal classified as a mammal, including humans, domestic and farm animals, zoo animals, animals used in sporting events (e.g., racehorses), or pet animals, such as dogs, horses, cats, cows, sheep, goats, pigs, etc. Preferably, the mammal is human.

The terms “peptide,” “polypeptide,” and “protein” are used interchangeably to refer to a polymer of amino acid residues, and are not limited to a minimum length, though a number of amino acid residues may be specified (e.g., 9mer is nine amino acid residues). Polypeptides may include amino acid residues including natural and/or non-natural amino acid residues. Polypeptides may also include fusion proteins. The terms also include post-expression modifications of the polypeptide, for example, glycosylation, sialylation, acetylation, phosphorylation, and the like. In some embodiments, the polypeptides may contain modifications with respect to a native or natural sequence, as long as the protein maintains the desired activity. These modifications may be deliberate, such as through site-directed mutagenesis, or may be accidental, such as through mutations of hosts which produce the proteins or errors due to PCR amplification.

The term “acidic residue” refers to amino acid residues in D- or L-form having sidechains comprising acidic groups. Exemplary acidic residues include D and E.

The term “amide residue” refers to amino acids in D- or L-form having sidechains comprising amide derivatives of acidic groups. Exemplary residues include N and Q.

The term “aromatic residue” refers to amino acid residues in D- or L-form having sidechains comprising aromatic groups. Exemplary aromatic residues include F, Y, and W.

The term “basic residue” refers to amino acid residues in D- or L-form having sidechains comprising basic groups. Exemplary basic residues include H, K, and R.

The term “hydrophilic residue” refers to amino acid residues in D- or L-form having sidechains comprising polar groups. Exemplary hydrophilic residues include C, S, T, N, and Q.

The term “nonfunctional residue” refers to amino acid residues in D- or L-form having sidechains that lack acidic, basic, or aromatic groups. Exemplary nonfunctional amino acid residues include M, G, A, V, I, L, and norleucine (Nle).

The term “neutral hydrophobic residue” refers to amino acid residues in D- or L-form having sidechains that lack basic, acidic, or polar groups. Exemplary neutral hydrophobic amino acid residues include A, V, L, I, P, W, M, and F.

The term “polar hydrophobic residue” refers to amino acid residues in D- or L-form having sidechains comprising polar groups. Exemplary polar hydrophobic amino acid residues include T, G, S, Y, C, Q, and N.

The term “hydrophobic residue” refers to amino acid residues in D- or L-form having sidechains that lack basic or acidic groups. Exemplary hydrophobic amino acid residues include A, V, L, I, P, W, M, F, T, G, S, Y, C, Q, and N.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally, or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include sulfur-containing: Met and Cysteine (Cys or C); acidic: Asp, Glu, Asn, and Gln; small aliphatic, nonpolar, or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company. Variant proteins, peptides, polypeptides, and amino acid sequences of the present disclosure can, in certain embodiments, comprise one or more conservative substitutions relative to a reference amino acid sequence.

“Nucleic acid molecule” or “polynucleotide” refers to a polymeric compound including covalently linked nucleotides comprising natural subunits (e.g., purine or pyrimidine bases). Purine bases include adenine (A) and guanine (G), and pyrimidine bases include uracil (U), thymine (T), and cytosine (C). Nucleic acid molecules include polyribonucleic acid (RNA) and polydeoxyribonucleic acid (DNA), which includes cDNA, genomic DNA, and synthetic DNA, either of which may be single or double-stranded. A nucleic acid molecule encoding an amino acid sequence includes all nucleotide sequences that encode the same amino acid sequence. In addition to the single letter codes for the nucleotides A, G, U, T, C, nucleotide sequences may use IUPAC single letter codes to designate more than one nucleotide alternative/ambiguous sequence, e.g., R (A or G); Y (C or T/U); M (A or C); K (G or T); S (C or G); W (A or T); H (A or C or T); B (C or G or T); V (A or C or G); D (A or G or T); or N (A or C or G or T).

The terms “nucleotide” and “nucleic acid” are used interchangeably and refer to any nucleoside linked to a phosphate group. Nucleic acids in accordance with the embodiments described herein may include nucleotides entirely of the types found in naturally occurring nucleic acids, or may instead include one or more nucleotide analogs or have a structure that otherwise differs from that of a naturally occurring nucleic acid. U.S. Patent. Nos. 6,403,779, 6,399,754, 6,225,460, 6,127,533, 6,031,086, 6,005,087, 5,977,089, disclose a wide variety of specific nucleotide analogs and modifications that may be used, and are hereby incorporated by reference as if fully set forth herein. Also see Crooke, S. Antisense Drug Technology: Principles, Strategies, and Applications (1st ed)., Marcel Dekker; ISBN: 0824705661; 1st edition (2001), which is also hereby incorporated by reference as if fully set forth herein. For example, the nucleoside may be natural, including but not limited to, any of cytidine, uridine, adenosine, guanosine, thymidine, inosine (hypoxanthine), or uric acid; or synthetic, including but not limited to methyl-substituted phenol analogs, hydrophobic base analogs, purine/pyrimidine mimics, icoC, isoG, thymidine analogs, fluorescent base analogs, or X or Y synthetic bases. Nucleic acids having a variety of different nucleotide analogs, modified backbones, or non-naturally occurring internucleoside linkages can be utilized in accordance with the embodiments described herein.

Nucleic acids may include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available.

Alternatively, a nucleotide may be abasic, such as but not limited to 3-hydroxy-2-hydroxymethyl-tetrahydrofuran, which act as a linker group lacking a base or be a nucleotide analog. 2′-modifications include halo, alkoxy and allyloxy groups, and the 2′-OH group can be replaced by a group selected from H, OR, R, halo, SH, SR, NH₂, NHR, NR₂ or CN, wherein R is C1-C6 alkyl, alkenyl, or alkynyl, and halo is F, Cl, Br, or I.

Non-limiting examples of synthetic bases and analogs include, but are not limited to methyl-substituted phenyl analogs, such as but not limited to mono-, di-, tri-, or tatramethylated benzene analogs; hydrophobic base analogs, such as but not limited to 7-propynyl isocarbostyril nucleoside, isocarbostyril nucleoside, 3-methylnapthalene, azaindole, bromo phenyl derivates at positions 2, 3, and 4, cyano derivatives at positions 2, 3, and 4, and fluoro derivates at position 2 and 3; purine/pyrimidine mimics, such as but not limited to azole hetercyclic carboxamides, such as but not limited to (1H)-1,2,3-triazole-4-carboxamide, 1,2,4-triazole-3-carboxamide, 1,2,3-triazole-4-carboxamide, or 1,2-pyrazole-3-carboxamide, or heteroatom-containing purine mimics, such as furo or theino pyridiones, such as but not limited to furo[2,3-c]pyridin-7(6H)-one, thieno[2,3-c]pyridin-7(6H)-one, furo[2,3-c]pyridin-7-thiol, furo[3,2-c]pyridin-4(5H)-one, thieno[3,2-c]pyridin-4(5H)-one, or furo[3,2-c]pyridin-4-thiol, or other mimics, such as but not limited to 5-phenyl-indolyl, 5-nitro-indolyl, 5-fluoro, 5-amino, 4-methylbenzimidazole, 6H,8H-3,4-dihydropropyrimido[4,5-c][1,2]oxazin-7-one, or N6-methoxy-2,6-diaminopurine; isocytosine, isoquanosine; thymidine analogs, such as but not limited to 5-methylisocytosine, difluorotoluene, 3-toluene-1-β-D-deoxyriboside, 2,4-difluoro-5-toluene-1-β-D-deoxyriboside, 2,4-dichloro-5-toluene-1-β-D-deoxyriboside, 2,4-dibromo-5-toluene-1-β-D-deoxyriboside, 2,4-diiodo-5-toluene-1-β-D-deoxyriboside, 2-thiothymidine, 4-Se-thymidine; or fluorescent base analogs, such as but not limited to 2-aminopurine, 1,3-diaza-2-oxophenothiazine, 1,3-diaza-2-oxophenoxazine, pyrrolo-dC and derivatives, 3-MI, 6-MI, 6-MAP, or furan-modified bases, phosporothioate nucleotides, 2′-O-methyl ribonucleotides, 2′-O-methoxy-ethyl ribonucleotides, peptide nucleotides, N3′-P5’ phosphoroamidate, 2′-fluoro-arabino nucleotides, locked nucleotides (LNA), unlocked nucleotides (UNA), morpholino phosphoroamidate, cyclohexene nucleotides, tricyclo-deoxynucleotides, or triazole-linked nucleotides. Examples of modified linkages include phosphorothioate and 5′-N-phosphoramidite linkages.

Modified nucleic acids need not be uniformly modified along the entire length of the molecule. Different nucleotide modifications and/or backbone structures may exist at various positions in the nucleic acid. The nucleotide analogs or other modification(s) may be located at any position(s) of a nucleic acid such that the function of the nucleic acid is not substantially affected. Non-limiting examples of modifications are located at any position of an aptamer component such that the ability of the aptamer to specifically bind to the target is not substantially affected. The modified region may be at the 5′-end and/or the 3′-end of one or both strands. For example, modified nucleic acid aptamers in which approximately 1-5 residues at the 5′ and/or 3′ end of either of both strands are nucleotide analogs and/or have a backbone modification have been employed. The modification may be a 5′ or 3′ terminal modification.

“Percent (%) sequence identity” with respect to a reference polypeptide sequence is the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN, or Megalign (DNASTAR) software, or other software appropriate for nucleic acid sequences. Appropriate parameters for aligning sequences are able to be determined, including algorithms to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a some % amino acid sequence identity to, with, or against a given amino acid sequence B) is calculated as follows: 100 times the fraction X/Y, where X is the number of amino acid residues scored as identical matches by the sequence alignment program ALIGN-2 in that program’s alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “nucleotide duplex” is when two strands of complement nucleotide oligomers complementary bind to each other. The two strands may be part of the same nucleotide molecule or separate nucleotide molecules.

“Ribonuclear protein” (RNP) is an association of an RNA-binding protein and a ribonucleic acid.

A “functional variant” refers to a polypeptide or polynucleotide that is structurally similar or substantially structurally similar to a parent or reference compound of this disclosure, but differs, in some contexts slightly, in composition (e.g., one base, atom, or functional group is different, added, or removed; or one or more amino acids are mutated, inserted, or deleted), such that the polypeptide or encoded polypeptide is capable of performing at least one function of the encoded parent polypeptide with at least 50% efficiency of activity of the parent polypeptide.

As used herein, a “functional portion” or “functional fragment” refers to a polypeptide or polynucleotide that comprises only a domain, motif, portion, or fragment of a parent or reference compound, and the polypeptide or encoded polypeptide retains at least 50% activity associated with the domain, portion, or fragment of the parent or reference compound. In certain embodiments, a functional portion refers to a “signaling portion” of an effector molecule, effector domain, costimulatory molecule, or costimulatory domain.

The term “expression,” as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

A “receptor” may be peptides, proteins, glycoproteins, lipoproteins, epitopes, antibodies, lipids, carbohydrates, multi-molecular structures, a specific conformation of one or more molecules and a morphoanatomic entity that has a binding affinity for a specific group of chemicals or molecules, such as other proteins or viruses. Upon recognition and binding of the chemical or molecule, the receptor can cause some form of signaling or other process within a cell to respond to the chemical or molecule. Optionally, the chemical or molecule can cause a receptor to stop functioning property and shut off processes.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence but need not be directly adjacent to it. For example, an enhancer is a transcriptional regulatory sequence that is operatively linked to a coding sequence, even though they are not contiguous.

As used herein, the terms “coding region” and “coding sequence” are used interchangeably and refer to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence-based amplification (NASBA, Canteen, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D. C. (1993). The product of amplification is termed an amplicon.

As used herein, “expression vector” refers to a nucleic acid construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. Vectors may be, for example, plasmids, cosmids, viruses, an RNA vector or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors). The terms should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; U.S. Pat. No. 5,591,439). Examples of non-viral vectors include, but are not limited to, liposomes, polyamine derivatives of DNA, and the like. Once transformed into a suitable host, the vector may replicate and function independently of the host genome, or may, in some instances, integrate into the genome itself. Here, “plasmid,” “expression plasmid,” “virus,” and “vector” are often used interchangeably. The terms refer broadly to any plasmid or virus encoding an exogenous nucleic acid.

As used herein “promoter” includes reference to a region of DNA upstream from the start of transcription and involved in recognition and binding of RNA polymerase and other proteins to initiate transcription.

The term “introduced” in the context of inserting a nucleic acid molecule into a cell means “transfection,” “transformation,” or “transduction” and includes reference to the incorporation of a nucleic acid molecule into a eukaryotic cell wherein the nucleic acid molecule may be incorporated into the genome of a cell and converted into an autonomous replicon. As used herein, the term “engineered,” “recombinant,” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications introducing expressible nucleic acid molecules encoding proteins, enzymes, or other nucleic acid molecule additions, deletions, substitutions, or other functional disruption of a cell’s genetic material.

The term “construct” refers to any polynucleotide that contains a recombinant nucleic acid molecule. A construct may be present in a vector (e.g., a bacterial vector, a viral vector) or may be integrated into a genome.

The term “host cell”, as used herein, includes any cell type which is susceptible to transformation with a nucleic acid construct. By “host cell” is meant a cell which contains a vector and supports the replication and/or expression of the vector. Host cells may be prokaryotic cells such as E. coli, or eukaryotic cells such as yeast, insect, amphibian, or mammalian cells.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

As used herein, the term “host” refers to a cell or microorganism targeted for genetic modification with a heterologous nucleic acid molecule to produce a polypeptide of interest. In certain embodiments, a host cell may optionally already possess or be modified to include other genetic modifications that confer desired properties related, or unrelated to, biosynthesis of the heterologous protein.

A “subject in need thereof” as used herein refers to a mammalian subject, preferably a human, who has been diagnosed with a condition, is suspected of having a condition, and/or exhibits one or more symptoms or risk factors associated with a condition.

The terms “treating” and “treatment” in relation to a given condition, disease, or disorder are used interchangeably and include, but are not limited to, inhibiting the disease or disorder, for example, arresting the development or rate of development of the condition, disease, or disorder; relieving the condition, disease, or disorder, for example, causing regression of the condition, disease, or disorder; or relieving a condition caused by or resulting from the disease or disorder, for example, arresting, relieving, preventing, or causing regression of at least one of the symptoms of the disease or disorder.

The terms “preventing” and “prevention” in relation to a given condition, disease, or disorder are used interchangeably and include, but are not limited to, preventing or delaying the onset of its development if none had occurred; preventing or delaying the condition, disease, or disorder from occurring in a subject that may be predisposed to the condition, disease, or disorder but has not yet been diagnosed as having the condition, disease; or disorder, and/or preventing or delaying further development of the condition, disease, or disorder if already present.

As used herein, “route” in relation to administration of one or more therapies, such as a therapeutic agent (e.g., drug), refers to a path by which the therapeutic agent is delivered to a subject, for example, a subject’s body. A route of therapeutic administration include enteral and parenteral routes of administration. Enteral administration includes oral, rectal, intestinal, and/or enema. Parenteral includes topical, transdermal, epidural, intracerebral, intracerebroventricular, epicutaneous, sublingual, sublabial, buccal, inhalational (e.g., nasal), intravenous, intraarticular, intracardiac, intradermal, intramuscular, intraocular, intraosseous infusion, intraperitoneal, intrathecal, intravitreal, subcutaneous, perivascular, implantation, vaginal, otic, and/or transmucosal.

While the present technology is capable of being embodied in various forms, the description below of several embodiments is made with the understanding that the present disclosure is to be considered as an exemplification of the technology and is not intended to limit the technology to the specific embodiments illustrated.

### Biosensors

Biosensors of the present technology generally comprise at least three regions, an aptamer region (e.g., aptamer), a reporter region (e.g., reporter), and a linker region (e.g., linker). Upon hybridization of a target with a target domain in the aptamer, the aptamer undergoes a conformational change (e.g., allosteric change) through a communication domain resulting in a change in signal emission, reduced or enhanced signal emission, by the reporter compared to a state when the aptamer is not bound to the target, i.e., as compared to a control. The emitted signal correlates to the presence of the target, such as identifying the target in the sample. In some embodiments, the emitted signal for the target (e.g., SARS-CoV2 spike protein) is less than a signal emitted by a control, such as a target in the same class yet having a different structure (e.g., MERS spike protein). In other embodiments, the emitted signal for the target is greater than a signal emitted by the control. Non-limiting examples of controls include phosphate buffered saline or a background signal generated by the fluorescent dye emitted at an emission wavelength followed by an excitation wavelength. Regardless of the embodiments, biosensors of the present technology are logic gated and binding to the target is compared to the control to determine presence or absence of the target in a sample which can be indicated by increased or decreased fluorescent signal upon target binding compared to the control.

In some embodiments, biosensors of the present technology are modular with one or more regions configured to be replaced with a different region of the same type. For example, a first aptamer region may be replaced with a second aptamer region that may or may not bind the same target as the first aptamer region. As another example, a first reporter region may be replaced with a second reporter region. This modular configuration of the biosensors of the present technology results in a plug-and-play design approach making the biosensors rapidly and efficiently adaptable for use in multiple different applications with minimal design change. In some embodiments, the biosensors are useful as a logic gate. Components of the biosensors, additional features of the biosensors, methods of preparing the biosensors, and methods of using the biosensors are described in greater detail below.

### Aptamers

Aptamers useful with biosensors of the present technology include at least three domains: a sensor domain configured to bind a target at a target binding site within the sensor domain, a reporter domain configured to bind a reporter at a reporter binding site within the reporter domain, and a linker. The aptamer is allosteric, resulting in a conformational change once the target binding site is bound to the target.

Aptamers useful with biosensors of the present technology include one or more strands of oligonucleotides including, but not limited to, RNA, DNA, PNA, LNA, or UNA. In some embodiments, the one or more strands of oligonucleotides comprise RNA. In other embodiments, one or more non-RNA nucleic acids, such as DNA, PNA, LNA, and UNA may be included in one or more strands of oligonucleotides to change a physical property of the biosensor, such as a rigidity of the biosensor. For example, UNA may be used to make a more relaxed backbone while LNA may make a more rigid backbone compared to a biosensor comprised solely of RNA.

The aptamer backbone may include one or more stems. In some embodiments, the aptamer backbone has a first stem formed from a first sequence within the backbone, also referred to herein as “stem 2”. In some embodiments, the aptamer backbone has a second stem formed from the 5′ and 3′ ends of the aptamer backbone, also referred to herein as the “outside stem”. According to some embodiments, the outside stem includes a first portion at the 5′ end and a second portion at the 3′ end. In some embodiments, the aptamer backbone has a third stem formed from a second sequence within the backbone, also referred to herein as the “inside stem”. In certain embodiments, the aptamer backbone has a sequence with an outside stem (OS) with a first portion (OS1) at the 5′ end and a second portion (OS2) at the 3′ end, an inside stem (IS), and a third stem (stem 2 or “S2”). For example the aptamer backbone can have a sequence 5′-OS1-CC-S2-GUGAUGGAG-IS-CAG-OS2-3′ according to some embodiments.

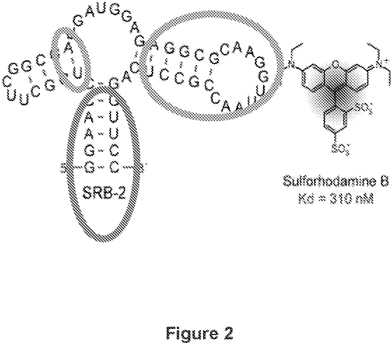

In some embodiments, the backbone is an aptamer having a publicly available sequence, such as but not limited to, SRB-2 (Bouhedda, et. al., and Holeman et al.). The nucleotide sequence of SRB-2 is 5′-GGAACCCCGCUUCGGCGGUG AUGGAGAGGCGCAAGGUUAACCGCCUCAGGUUCC-3′ (SEQ ID N0:103) (“the aptamer backbone”), where “stem 2” of the aptamer backbone is italicized and bolded, the “inside stem” of the aptamer backbone is underlined and italicized, and the “outside stem” of the aptamer backbone is in bold. In certain embodiments, the C-G base pair underlined in the aptamer backbone (“the C-G base pair”) may be replaced by a U-A base pair (“the U-A base pair”) as shown in FIG. 2 (circled). In other embodiments, the C-G base pair underlined in the aptamer backbone may be any suitable base pair in any order (i.e., an N-N base pair such as U-A, A-U, C-G, G-C, etc.). In other embodiments, the backbone is an aptamer having a sequence that is not publicly known.

Target domains of the aptamers include randomized regions at one or more stems of the backbone. Without intending to be limiting, the randomized regions of the target domains are thought to confer specificity for the target bound by the target domain. As described herein, SELEX can be used to identify aptamers useful for binding to a particular target domain.

The length of the randomized region can be selected based on a size of the target that the aptamer is sought to bind and having a desired signal intensity after binding to the target. For example, smaller targets, such as short oligonucleic acids, short peptides, and ions, may be bound by aptamers having shorter randomized regions (e.g., under 40 nucleic acids). As another example, larger targets, such as larger proteins (e.g., spike proteins) may be bound by aptamers having longer randomized regions (e.g., more than 40 nucleic acids). In some embodiments, randomized regions include at least 10, at least 20, at least 30, at least 40, at least 50, or at least 60 nucleotides, for example, the randomized region is about 15 nucleotides, about 20 nucleotides, about 25 nucleotides, about 30 nucleotides, about 35 nucleotides, about 40 nucleotides, about 45 nucleotides, about 50 nucleotides, about 55 nucleotides, about 60 nucleotides, or more than 60 nucleotides. Non-limiting examples of randomized region nucleotide lengths are about 15 (e.g., NNNNNNNNNNNNNNN (SEQ ID NO:160), or “N15”), about 30 (e.g., NNNNNNNNNNNNNNNNNNNNNNNNNNNNNN (SEQ ID NO:162), or “N30”), about 45, (e.g., NNNNNNNNNNNNNNNNNNNNNNNNNNNNNNNNNNNNNNNNNNNNN (SEQ ID NO:163), or “N45”), about 46 (e.g., NNNNNNNNNNNNNNNNNNNNN NNNNNNNNNNNNNNNNNNNNNNNNN (SEQ ID NO:164), or “N46”), or about 60 (e.g., NNNNNNNNNNNNNNNNNNNNNNNNNNNNNNNNNNNNNNNNNNNNNNNNNNNNNNN NNNNN (SEQ ID NO:131), or “N60”) nucleotides. In certain aspects, the randomized regions may be split into two separate regions as shown in the nucleotide sequences herein. Non-limiting examples include a randomized region including about 60 nucleotides may be indicated by a nucleotide with two 30 nucleotide (N30) regions; a randomized region including about 46 nucleotides may be indicated by a nucleotide with two 23 nucleotide (N23) regions (NNNNNNNNNNNNNNNNNNNNNNN; SEQ ID NO:161); or a randomized region including about 30 nucleotides may be indicated by a nucleotide with two 15 nucleotide (N15) regions.

The randomized regions can be located (e.g., inserted) on any stem of the aptamer backbone and at any location within the stem. (Legiewicz et al.) In certain embodiments, the randomized region(s) can replace or be inserted at the nucleotides of the stem. For example, in some embodiments, randomized regions can replace or be inserted at a first stem (e.g., stem 2) of the aptamer backbone (with the U-A base pair). The position of stem 2 of the aptamer backbone is illustrated in the embodiment shown in FIG. 1. In certain embodiments, a randomized region replaces the first stem at position “N_{Y}” (underlined) of the following sequence having the U-A base pair: GGAACCUN_{Y}AUGAUGGAGAGGCGCAAGGUUAACCGCCUCAGGUUCC (SEQ ID NO:104). In other embodiments, a randomized region replaces the first stem at position “N_{Y}” (underlined) of the following sequence having the C-G base pair: GGAACCCN_{Y}GUGAUGGAGAGGCGCAAGGUUAACCGCCUCAGGUUCC (SEQ ID NO:105). In other embodiments, a randomized region replaces the first stem at position “N_{Y}” (underlined) of the following sequence having the N-N base pair: GGAACCNN_{Y}NUGAUGGAGAGGCGCAAGGUUAACCGCCUCAGGUUCC (SEQ ID NO:106). In the aforementioned sequences the _{Y} represents the number of randomized nucleotides (N) that replace or are otherwise inserted at that position. In some embodiments, _{Y} is any number between 10 and 70. In certain embodiments, N_{Y} is N15, N30, N45, N46, or N60.

Non-limiting exemplary sequences where the randomized region replaces the first stem include a sequence where N_{Y} is N30 (e.g., GGAACCUNNNNNNNNNNNNNNNNNNNNNNNNNNNNNNAUGAUGGAGAGGCGCAAG GUUAACCGCCUCAGGUUCC (SEQ ID NO:107), GGAACCCNNNNNNNNNNNNN NNNNNNNNNNNNNNNNNGUGAUGGAGAGGCGCAAGGUUAACCGCCUCAGGUUCC (SEQ ID N0:108), GGAACCNNNNNNNNNNNNNNNNNNNNNNNNNNNNNNNNUG AUGGAGAGGCGCAAGGUUAACCGCCUCAGGUUCC) (SEQ ID N0:109)); where N_{Y} is N45 (e.g., GGAACCUNNNNNNNNNNNNNNNNNNNNNNNNNNNNNNNNNNNNNNNN NNNNNAUGAUGGAGAGGCGCAAGGUUAACCGCCUCAGGUUCC (SEQ ID NO:110), GGAACCCNNNNNNNNNNNNNNNNNNNNNNNNNNNNNNNNNNNNNNNNNNNNNGUG AUGGAGAGGCGCAAGGUUAACCGCCUCAGGUUCC (SEQ ID NO:111), GGA ACCNNNNNNNNNNNNNNNNNNNNNNNNNNNNNNNNNNNNNNNNNNNNNNNUGAUG GAGAGGCGCAAGGUUAACCGCCUCAGGUUCC (SEQ ID NO:112)), or where N_{Y} is N60 (e.g., GGAACCUNNNNNNNNNNNNNNNNNNNNNNNNNNNNNNNNNNNNNNNNNNNN NNNNNNNNNNNNNNNNAUGAUGGAGAGGCGCAAGGUUAACCGCCUCAGGUUCC (SEQ ID NO:113), GGAACCCNNNNNNNNNNNNNNNNNNNNNNNNNNNNNNNN NNNNNNNNNNNNNNNNNNNNNNNNNNNNGUGAUGGAGAGGCGCAAGGUUAACCGC CUCAGGUUCC (SEQ ID NO:114), GGAACCNNNNNNNNNNNNNNNNNNN NNNNNNNNNNNNNNNNNNNNNNNNNNNNNNNNNNNNNNNNNNNUGAUGGAGAGGC GCAAGGUUAACCGCCUCAGGUUCC (SEQ ID NO:115)).

In another embodiment, one or more randomized regions can replace or be inserted in the outside stem of the aptamer backbone having the U-A or C-G base pair; or in the inside stem of the aptamer backbone having the U-A or at C-G base pair. The positions of the outside stem and inside stem of the aptamer backbone is illustrated in the embodiment shown in FIG. 2. In certain embodiments, a randomized region replaces the outside stem at positions “N_{Y}” (underlined) of the following sequence having the U-A base pair: N_{Y}CUCGCUUCGGCGAUGAUGGAGAGGCGCAAGGUUAACCGCCUCAGN_{Y} (SEQ ID NO:116). In certain embodiments, a randomized region replaces the outside stem at positions “N_{Y}” (underlined) of the following sequence having the C-G base pair: N_{Y}CCCGCUUCGGCGGUGAUGGAGAGGCGCAAGGUUAACCGCCUCAGN_{Y} (SEQ ID NO:117). In certain embodiments, a randomized region replaces the outside stem at positions “N_{Y}” (underlined) of the following sequence having the N-N base pair: N_{Y}CNCGCUUCGGCGNUGAUGGAGAGGCGCAAGGUUAACCGCCUCAGN_{Y} (SEQ ID NO:118). In other embodiments, a randomized region replaces the inside stem at positions “N_{Y}” (underlined) of the following sequence having the U-A base pair: GGAACCUCGCUUCGGCGAUGAUGGAGN_{Y}CAGGUUCC (SEQ ID NO:119). In other embodiments, a randomized region replaces the inside stem at positions “N_{Y}” (underlined) of the following sequence having the C-G base pair: GGAACCCCGCUUCGGCGGUGAUGGAGN_{Y}CAGGUUCC (SEQ ID NO:120). In other embodiments, a randomized region replaces the inside stem at positions “N_{Y}” (underlined) of the following sequence having the N-N base pair: GGAACCNCGCUUCGGCGNUGAUGGAGN_{Y}CAGGUUCC (SEQ ID NO:121). In the aforementioned sequences, the _{Y} represents the number of randomized nucleotides (N) that replace or are otherwise inserted at that position. In some embodiments, N_{Y} is any number of randomized nucleotides between 10 and 70. In certain embodiments, N_{Y} is 15 (N15), 23 (N23), 30 (N30), 45 (N45), 46 (N46), or 60 (N60).

In some embodiments, the randomized region includes 30 nucleotides. A non-limiting exemplary sequence for a randomized region of 30 nucleotides (i.e., N15 at the 5′ end and N15 at the 3′ end) that replaces or is inserted at the outside stem of the aptamer backbone having the U-A base pair (underlined) is: NNNNNNNNNNNNNNNCUCGCUUCGGCGAUGAUGGAGAGGCGCAAGGUUAACCGCC UCAGNNNNNNNNNNNNNNN (SEQ ID NO:122). A non-limiting exemplary sequence for a randomized region of 30 nucleotides (i.e., N15 at the 5′ end and N15 at the 3′ end) that replaces or is inserted at the outside stem of the aptamer backbone having the C-G base pair (underlined) is: NNNNNNNNNNNNNNNCCCGCUUCGGCGGUGAU GGAGAGGCGCAAGGUUAACCGCCUCAGNNNNNNNNNNNNNNN (SEQ ID NO:123). A non-limiting exemplary sequence for a randomized region of 30 nucleotides (i.e., N15 at the 5′ end and N15 at the 3′ end) that replaces or is inserted at the outside stem of the aptamer backbone having the N-N base pair (underlined) is: NNNNNNNNNNNNNNNCNCGCUUCGGCGNUGAUGGAGAGGCGCAAGGUUAACCGCC UCAGNNNNNNNNNNNNNNN (SEQ ID NO:124). A non-limiting exemplary sequence for a randomized region of 30 nucleotides (i.e., N30) that replaces or is inserted at the inside stem of the aptamer backbone having the U-A base pair (underlined) is: GGAACCUCGCUUCGGCGAUGAUGGAGNNNNNNNNNNNNNNNNNNNNNNNNNNNNN NCAGGUUCC (SEQ ID N0:125). A non-limiting exemplary sequence for a randomized region of 30 nucleotides (i.e., N30) that replaces or is inserted at the inside stem of the aptamer backbone having the C-G base pair (underlined) is: GGAACCCCGCUUCGGCGGUGAUGGAGNNNNNNNNNNNNNNNNNNNNNNNNNNNNN NCAGGUUCC (SEQ ID N0:126). A non-limiting exemplary sequence for a randomized region of 30 nucleotides (i.e., N30) that replaces or is inserted at the inside stem of the aptamer backbone having the N-N base pair (underlined) is: GGAACCNCGCUUCGGCGNUGAUGGAGNNNNNNNNNNNNNNNNNNNNNNNNNNNNN NCAGGUUCC (SEQ ID NO:127).

In some embodiments, the randomized region includes 46 nucleotides. A non-limiting exemplary sequence for a randomized region of 46 nucleotides (i.e., N23 at the 5′ end and N23 at the 3′ end) that replaces or is inserted at the outside stem of the aptamer backbone having the U-A base pair (underlined) is: NNNNNNNNNNNNNNNNNNNNNNNCUCGCUUCGGCGAUGAUGGAGAGGCGCAAGGU UAACCGCCUCAGNNNNNNNNNNNNNNNNNNNNNNN (SEQ ID NO:128). A non-limiting exemplary sequence for a randomized region of 46 nucleotides (i.e., N23 at the 5′ end and N23 at the 3′ end) that replaces or is inserted at the outside stem of the aptamer backbone having the C-G base pair (underlined) is: NNNNNNNNNNNNNNNNNNN NNNNCCCGCUUCGGCGGUGAUGGAGAGGCGCAAGGUUAACCGCCUCAGNNNNNNN NNNNNNNNNNNNNNNN (SEQ ID NO:129). A non-limiting exemplary sequence for a randomized region of 46 nucleotides (i.e., N23 at the 5′ end and N23 at the 3′ end) that replaces or is inserted at the outside stem of the aptamer backbone having the N-N base pair (underlined) is: NNNNNNNNNNNNNNNNNNNNNNNCNCGCUUCGGCGNU GAUGGAGAGGCGCAAGGUUAACCGCCUCAGNNNNNNNNNNNNNNNNNNNNNNN (SEQ ID NO:130).

In some embodiments, the randomized region includes 45 nucleotides. A non-limiting exemplary sequence for a randomized region of 45 nucleotides (i.e., N45) that replaces or is inserted at the inside stem of the aptamer backbone having the U-A base pair (underlined) is: GGAACCUCGCUUCGGCGAUGAUGGAGNNNNNNNNNNNNNNNNN NNNNNNNNNNNNNNNNNNNNNNNNNNNNCAGGUUCC (SEQ ID NO:131). A non-limiting exemplary sequence for a randomized region of 45 nucleotides (i.e., N45) that replaces or is inserted at the inside stem of the aptamer backbone having the C-G base pair (underlined) is: GGAACCCCGCUUCGGCGGUGAUGGAGNNNNNNNNNNNNNNNNNNN NNNNNNNNNNNNNNNNNNNNNNNNNNCAGGUUCC (SEQ ID NO:132). A non-limiting exemplary sequence for a randomized region of 45 nucleotides (i.e., N45) that replaces or is inserted at the inside stem of the aptamer backbone having the N-N base pair (underlined) is: GGAACCNCGCUUCGGCGNUGAUGGAGNNNNNNNNNNNNNNNNNNNNNNNNNN NNNNNNNNNNNNNNNNNNNCAGGUUCC (SEQ ID NO:133).

In some embodiments, the randomized region includes 60 nucleotides. A non-limiting exemplary sequence for a randomized region of 60 nucleotides (i.e., N30 at the 5′ end and N30 at the 3′ end) that replaces or is inserted at the outside stem of the aptamer backbone having the U-A base pair (underlined) is: NNNNNNNNNNNNNNNNNNNNNNNNNNNNNNCUCGCUUCGGCGAUGAUGGAGAGGC GCAAGGUUAACCGCCUCAGNNNNNNNNNNNNNNNNNNNNNNNNNNNNNN (SEQ ID NO:134). A non-limiting exemplary sequence for a randomized region of 60 nucleotides (i.e., N30 at the 5′ end and N30 at the 3′ end) that replaces or is inserted at the outside stem of the aptamer backbone having the C-G base pair (underlined) is: NNNNNNNNNNNNNNNNNNNNNNNNNNNNNNCCCGCUUCGGCGGUGAUGGAGAGGC GCAAGGUUAACCGCCUCAGNNNNNNNNNNNNNNNNNNNNNNNNNNNNNN (SEQ ID NO:135). A non-limiting exemplary sequence for a randomized region of 60 nucleotides (i.e., N30 at the 5′ end and N30 at the 3′ end) that replaces or is inserted at the outside stem of the aptamer backbone having the N-N base pair (underlined) is: NNNNNNNNNNNNNNNNNNNNNNNNNNNNNNCNCGCUUCGGCGNUGAUGGAGAGGC GCAAGGUUAACCGCCUCAGNNNNNNNNNNNNNNNNNNNNNNNNNNNNNN (SEQ ID NO:136). A non-limiting exemplary sequence for a randomized region of 60 nucleotides (i.e., N60) that replaces or is inserted at the inside stem of the aptamer backbone having the U-A base pair (underlined) is: GGAACCUCGCUUCGGCGAUG AUGGAGNNNNNNNNNNNNNNNNNNNNNNNNNNNNNNNNNNNNNNNNNNNNNNNNN NNNNNNNNNNNCAGGUUCC (SEQ ID NO:137). A non-limiting exemplary sequence for a randomized region of 60 nucleotides (i.e., N60) that replaces or is inserted at C-G in the inside stem of the aptamer backbone having the C-G base pair (underlined) is: GGAACCCCGCUUCGGCGGUGAUGGAGNNNNNNNNNNNNNNNNNNNNNNNNNNNNN NNNNNNNNNNNNNNNNNNNNNNNNNNNNNNNCAGGUUCC (SEQ ID NO:138). A non-limiting exemplary sequence for a randomized region of 60 nucleotides (i.e., N60) that replaces or is inserted at N-N in the inside stem of the aptamer backbone having the C-G base pair (underlined) is: GGAACCNCGCUUCGGCGNUGAUGGAGNNNN NNNNNNNNNNNNNNNNNNNNNNNNNNNNNNNNNNNNNNNNNNNNNNNNNNNNNNN NCAGGUUCC (SEQ ID NO:139).

Non-limiting examples of nucleotide sequences that encode RNA aptamers of a biosensor in accordance with the embodiments described herein are provided in Table 1 below. The sequences in Table 1 include (i) forward primer sequences (F-primer, indicated by bold italic text), and/or (ii) a reverse primer sequence (R-primer, indicated by bold text), and/or (iii) a T7 promoter sequence (indicated by italic text) useful with the present technology, and may be used to produce exemplary RNA aptamers with randomized region(s) within the backbone in accordance with the embodiments described herein and described further below.

TABLE 1

Nucleotide Sequences

| | | | |
|---|---|---|---|
| ID | NAME | PCR PRIMERS | SEQUENCE |
| RHO-35 | DAF2R2R5-1 | Rho-1/3 | ATGCATGTCCGGACTGCCACCTGCAAGCCACGC GCTGAGGCGGTTAACCTTGCGCCTCTCCATCATC GCCGAAGCGAGCGCGGGCAGTAGCCGCAGGTAC ACCGGGCAGGGCTACCTATAGTGAGTCGTATTAT CGGC (SEQ ID NO:35) |
| RHO-36 | DAF2R2R5-1b | | CCTGCAAGCCACGCGCTGAGGCGGTTAACCTTGC GCCTCTCCATCATCGCCGAAGCGAGCGCGGGCA GTAGCCGTATAGTGAGTCGTATTATCGGC (SEQ ID NO:36) |
| RHO-37 | DAF2R2R5-2 | Rho-1/3 | ATGCATGTCCGGACTGCCAGGTGCACCGATTGGT CTGAGGCGGTTAACCTTGCGCCTCTCCATCATCG CCGAAGCGAGAACCAATTGCGAGAGCAGGTACA CCGGGCAGGGCTACCTATAGTGAGTCGTATTATC GGC (SEQ ID NO:37) |
| RHO-38 | DAF2R2R5-2b | | GGTGCACCGATTGGTCTGAGGCGGTTAACCTTGC GCCTCTCCATCATCGCCGAAGCGAGAACCAATTG CGAGAGTATAGTGAGTCGTATTATCGGC (SEQ ID NO:38) |
| RHO-39 | DAF2R2R5-3 | Rho-1/3 | ATGCATGTCCGGACTGCCAGAACGGTAGTTAGTG CTGAGGCGGTTAACCTTGCGCCTCTCCATCATCG CCGAAGCGAGGCAATTAACTTGGTACAGGTACAC CGGGCAGGGCTACCTA TAGTGAGTCGTA ITA TCG GC (SEQ ID NO:39) |
| RHO-40 | DAF2R2R5-3b | | GAACGGTAGTTAGTGCTGAGGCGGTTAACCTTGC GCCTCTCCATCATCGCCGAAGCGAGGCAATTAAC TTGGTATATAGTGAGTCGTATTATCGGC (SEQ ID NO:40) |
| RHO-43 | DAF2R2R5-5 | Rho-1/3 | ATGCATGTCCGGACTGCCATCCGAAACGGGCACT CTGAGGCGGTTAACCTTGCGCCTCTCCATCATCG CCGAAGCGAGGAGTGCCAGCTGCCCCAGGTACA CCGGGCAGGGCTACCTATAGTGAGTCGTATTATC GGC (SEQ ID NO:43) |
| RHO-44 | DAF2R2R5-5b | | TCCGAAACGGGCACTCTGAGGCGGTTAACCTTGC GCCTCTCCATCATCGCCGAAGCGAGGAGTGCCAG CTGCCCTATAGTGAGTCGTATTATCGGC (SEQ ID NO:44) |
| RHO-45 | DAF2R2R5-6 | Rho-1/3 | ATGCATGTCCGGACTGCCAATCCGAACGGCGGA TCTGAGGCGGTTAACCTTGCGCCTCTCCATCATC GCCGAAGCGAGGATCCGCATACTGCCCAGGTAC ACCGGGCAGGGCTACCTATAGTGAGTCGTATTAT CGGC (SEQ ID NO:45) |
| RHO-46 | DAF2R2R5-6b | | ATCCGAACGGCGGATCTGAGGCGGTTAACCTTGC GCCTCTCCATCATCGCCGAAGCGAGGATCCGCAT ACTGCCTATAGTGAGTCGTATTATCGGC (SEQ ID NO:46) |
| RHO-47 | DAF2R2R5-7 | Rho-1/3 | ATGCATGTCCGGACTGCCACGATGACCCGCAGA GCTGAGGCGGTTAACCTTGCGCCTCTCCATCATC GCCGAAGCGAGGCTCAATTCAAAACGCAGGTACA CCGGGCAGGGCTACCTATAGTGAGTCGTATTATC GGC (SEQ ID NO:47) |
| RHO-48 | DAF2R2R5-8 | Rho-1/3 | ATGCATGTCCGGACTGCCAGGTGTACGAATGAGC CTGAGGCGGTTAACCTTGCGCCTCTCCATCATCG CCGAAGCGAGGGCTCAACTGTAGAACAGGTACA CCGGGCAGGGCTACCTATAGTGAGTCGTATTATC GGC (SEQ ID NO:48) |
| RHO-49 | DAF2R2R5-9 | Rho-1/3 | ATGCATGTCCGGACTGCCAGGGGTACCTGAGCT CCTGAGGCGGTTAACCTTGCGCCTCTCCATCATC GCCGAAGCGAGGAGCTTGGTAACGTTCAGGTAC ACCGGGCAGGGCTACCTATAGTGAGTCGTATTAT CGGC (SEQ ID NO:49) |
| RHO-50 | DAF2R2R5-10 | Rho-1/3 | ATGCATGTCCGGACTGCCAGGTGTGCCAACGCG GCTGAGGCGGTTAACCTTGCGCCTCTCCATCATC GCCGAAGCGAGACCGCGTGGTAGGATCAGGTAC ACCGGGCAGGGCTACCTATAGTGAGTCGTATTAT CGGC (SEQ ID NO:50) |
| RHO-51 | DAF2R2R5-11 | Rho-1/3 | ATGCATGTCCGGACTGCCAGGGTACCCGGGACG CCTGAGGCGGTTAACCTTGCGCCTCTCCATCATC GCCGAAGCGAGGCGTCGATTTAGTAGCAGGTAC ACCGGGCAGGGCTACCTATAGTGAGTCGTATTAT CGGC (SEQ ID NO:51) |
| RHO-52 | DAF2R2R5-1b-R-primer | | CCTGCAAGCCACGCGCTGAGGCGG (SEQ ID NO:52) |
| RHO-53 | DAF2R2R5-1b-FT7-primer | | GCCGATAATACGACTCACTATACGGCTACTGCCC GCGCTCGC (SEQ ID NO:53) |
| RHO-54 | DAF2R2R5-11b-R-primer | | GGGTACCCGGGACGCCTGAGGC (SEQ ID NO:54) |
| RHO-55 | DAF2R2R5-11b-FT7-primer | | GCCGATAATACGACTCACTATACTACTAAATCGAC GCCTCGCTTCGGC (SEQ ID NO:55) |
| RHO-56 | Rho-28-DNA | | GGAACCTGAAATACGTGTCCATATCCGCAATCCC GAGCAGCCCCTTTTGGACCCGATGAAATGCTGCG TCACTCTGGATAAATCTCCATCATCGCCGAAGCGA GGTTCC (SEQ ID NO:56) |
| RHO-57 | RHO-56-reverse-primer | | GGAACCTGAAATACGTGTCCATATCC (SEQ ID NO:57) |
| RHO-58 | RHO-56/59-forward-T7 | | GCCGA TAA TACGACTCACTA TAGGAA CCTCGCTT CGGCG (SEQ ID NO:58) |
| RHO-59 | Rho-31-DNA | | GGAACCTGAAATTGTCCATTAACGCCCTTGGACC AGCACTCCCAAAGCACAAGGTCGGTGCTGAAATC TCCATCACCGCCGAAGCGGGGTTCC (SEQ ID NO:59) |
| RHO-60 | RHO-59-reverse primer | | GGAACCTGAAATTGTCCATTAACGCC (SEQ ID NO:60) |
| RHO-61 | originalSRONapt amer-primers | Rho-1/3 | ATGCATGTCCGGACTGCCAGGAACCTGAGGCGG TTAACCTTGCGCCTCTCCATCATCGCCGAAGCGA GGTTCCCAGGTACACCGGGCAGGGCTACCTATA GTGAGTCGTATTATCGGC (SEQ ID NO:61) |
| RHO-62 | originalSRDNapt amer-noprimers | Rho-2 | GGAACCTGAGGCGGTTAACCTTGCGCCTCTCCAT CATCGCCGAAGCGAGGTTCCTATAGTGAGTCGTA TTATCGGC (SEQ ID NO:62) |
| RHO-63 | DAF1-4560-R6-63 | Rho-10/11 | ATGCGCAGGACCCGGTGAGCCAGCCCTAGGGCG GGGCTTCCTTGGTTATCGTCTGAGGCGGTTAACC TTGCGCCTCTCCATCAGCGCCGAAGCGCGACGAC AGGATGCTGAGGGCGGACAGGTTGGCAGGCCTA GTCGTAGCCTAGCATCGCCTATAGTGAGTCGTAT TATCGGC (SEQ ID NO:63) |
| RHO-64 | DAF2-4560-R6-70 | Rho-10/11 | ATGCGCAGGACCCGGTGAGCCATCTTCGGTGCA GCCGCGTAGCAGCTGAGGCGGTTAACCTTGCGC CTCTCCATCAGCGCCGAAGCGAGAGCCTTGACTA CGATTAGGTTGGCAGGCCTAGTCGTAGCCTAGCA TCGCCTATAGTGAGTCGTATTATCGGC (SEQ ID NO:64) |
| RHO-65 | DAF1-4560-65 | Rho-10/11 | ATGCGCAGGACCCGGTGAGCCACACGGCGGTAC GGGCACGGTATCCTGAGGCGGTTAACCTTGCGCC TCTCCATCAGCGCCGAAGCGCGGCATAATCGATT AGGCCCGGGCGCAGGCCTAGTCGTAGCCTAGCA TCGCCTATAGTGAGTCGTATTATCGGC (SEQ ID NO:65) |
| RHO-66 | DAF1-4560-1 | Rho-10/11 | ATGCGCAGGACCCGGTGAGCCAGCGGGCACTCA AATCGATCAAATCTGAGGCGGTTAACCTTGCGCC TCTCCATCAGCGCCGAAGCGCGAAACGATCGTTT GCGGAGCTACTCAGGCCTAGTCGTAGCCTAGCA TCGCCTATAGTGAGTCGTATTATCGGC (SEQ ID NO:66) |
| RHO-67 | DAF1-4560-2 | Rho-10/11 | ATGCGCAGGACCCGGTGAGCCAACGGGGGCAAT CTGCGTCGCAGCCTGAGGCGGTTAACCTTGCGCC TCTCCATCAGCGCCGAAGCGCGGCCACTGACGA CGTAGGTCGCAACAGGCCTAGTCGTAGCCTAGC ATCGCCTATAGTGAGTCGTATTATCGGC (SEQ ID NO:67) |
| RHO-68 | DAF_1_2N-4560-68 | Rho-10/11 | ATGCGCAGGACCCGGTGAGCCAGACGGATCAAT GGCCTGTGTGGACTGAGGCGGTTAACCTTGCGCC TCTCCATCAGCGCCGAAGCGCGTACCACCGTAGG TGAGGTTTACTCAGGCCTAGTCGTAGCCTAGCAT CGCCTATAGTGAGTCGTATTATCGGC (SEQ ID NO:68) |
| RHO-69 | DAF2-4560-71 | Rho-10/11 | ATGCGCAGGACCCGGTGAGCCACGTGGCAGAGG TTTACCATCGGACCGGGAGCTGAGGCGGTTAACC TTGCGCCTCTCCATCAGCGCCGAAGCGCGAGCTC CATGCGAAGGCTACGGACGAGGTGGCAGGCCTA GTCGTAGCCTAGCATCGCCTATAGTGAGTCGTAT TATCGGC (SEQ ID NO:69) |
| RHO-70 | DAF2-4560-1 | Rho-10/11 | ATGCGCAGGACCCGGTGAGCCATTAGCGAATGA TCACACGGGTGACTGAGGCGGTTAACCTTGCGCC TCTCCATCAGCGCCGAAGCGAGGCCAGCGACCTT GGCCGTCGACCCAGGCCTAGTCGTAGCCTAGCA TCGCCTATAGTGAGTCGTATTATCGGC (SEQ ID NO:70) |
| RHO-71 | DAF2-30-76 | Rho-1/3 | ATGCATGTCCGGACTGCCAGATGTACCAGCGCAA CTGAGGCGGTTAACCTTGCGCCTCTCCATCATCG CCGAAGCGAGGTTGCGCGTTGGATTCAGGTACA CCGGGCAGGGCTACCTATAGTGAGTCGTATTATC GGC (SEQ ID NO:71) |
| RHO-72 | DAF2-30-1 | Rho-1/3 | ATGCATGTCCGGACTGCCACGGTGTACAACAGG GCTGAGGCGGTTAACCTTGCGCCTCTCCATCATC GCCGAAGCGAGGCTCTGCGTCTTTTTCAGGTACA CCGGGCAGGGCTACCTATAGTGAGTCGTATTATC GGC (SEQ ID NO:72) |
| RHO-73 | DAF2-30-78 | Rho-1/3 | ATGCATGTCCGGACTGCCAGGTGTACCATAGACG CTGAGGCGGTTAACCTTGCGCCTCTCCATCATCG CCGAAGCGAGGCGTCTACAGAAAAGCAGGTACA CCGGGCAGGGCTACCTATAGTGAGTCGTATTATC GGC (SEQ ID NO:73) |
| RHO-74 | DAF2-30-79 | Rho-1/3 | ATGCATGTCCGGACTGCCAGGCATAACTGATGTG CTGAGGCGGTTAACCTTGCGCCTCTCCATCATCG CCGAAGCGAGCACATTGGTAACGTTCAGGTACAC CGGGCAGGGCTACCTA TAGTGAGTCGTA ITA TCG GC (SEQ ID NO:74) |
| RHO-75 | DAF2-30-2 | Rho-1/3 | ATGCATGTCCGGACTGCCACGTCCTGCCACCTGT CTGAACTGAGGCGGTTAACCTTGCGCCTCTCCAT CATCGCCGAAGCGAGTTTCAGACATCGGGGCAG GTA CA CCGGGCA GGGCTA CCTA TAGTGAGTCGTA TTATCGGC (SEQ ID NO:75) |
| RHO-76 | DAF2-30-3 | Rho-1/3 | ATGCATGTCCGGACTGCCAGGAACCTGTTAACCT CGTGGTAGCATCGGCGAGTGGAACTCCATCATCG CCGAAGCGAGGTTCCCAGGTACACCGGGCAGGG CTACCTATAGTGAGTCGTATTATCGGC (SEQ ID NO:76) |
| RHO-77 | DAF1-30-85 | Rho-1/3 | ATGCATGTCCGGACTGCCAGGAACCTGGGAAAC CGCGTTACAACGAGATGGGGTAACCTCCATCATC GCCGAAGCGAGGTTCCCAGGTACACCGGGCAGG GCTACCTATAGTGAGTCGTATTATCGGC (SEQ ID NO:77) |
| RHO-78 | DAF1-30-1 | Rho-1/3 | ATGCATGTCCGGACTGCCAAGCGCACCGGCCTT ACTGAGGCGGTTAACCTTGCGCCTCTCCATCATC GCCGAAGCGAGTAAGTCGATCGAGATCAGGTACA CCGGGCAGGGCTACCTATAGTGAGTCGTATTATC GGC (SEQ ID NO:78) |
| RHO-79 | DAF1-30-80 | Rho-1/3 | ATGCATGTCCGGACTGCCAAGTGTACCGACTGTG CTGAGGCGGTTAACCTTGCGCCTCTCCATCATCG CCGAAGCGAGCACAGTGATAGCTTTCAGGTACAC CGGGCAGGGCTACCTATAGTGAGTCGTAITATCG GC (SEQ ID NO:79) |
| RHO-80 | DAF1-30-81 | Rho-1/3 | ATGCATGTCCGGACTGCCAGGTGTACGAATCGCA CTGAGGCGGTTAACCTTGCGCCTCTCCATCATCG CCGAAGCGAGTGCGATCGGATAGCTCAGGTACA CCGGGCAGGGCTACCTATAGTGAGTCGTATTATC GGC (SEQ ID NO:80) |
| RHO-81 | DAF1-30-82 | Rho-1/3 | ATGCATGTCCGGACTGCCAAGTGTACCTCTTCCG CTGAGGCGGTTAACATTGCGCCTCTCCATCACCG CCGAAGCGGGCGGAAGCCTTAGCCACAGGTACA CCGGGCAGGGCTACCTATAGTGAGTCGTATTATC GGC (SEQ ID NO:81) |
| RHO-82 | DAF1-4560c-1 | Rho-10/11 | ATGCGCAGGACCCGGTGAGCCAGTCGAGCCCTA GCTCATTGCGTACTGAGGCGGTTAACCTTGCGCC TCTCCATCAGCGCCGAAGCGCGTACGCGTAGTGA TGAGTACGACTCAGGCCTAGTCGTAGCCTAGCAT CGCCTATAGTGAGTCGTATTATCGGC (SEQ ID NO:82) |
| RHO-83 | DAF1-4560c-2 | Rho-10/11 | ATGCGCAGGACCCGGTGAGCCAAGCCGAGTGAT GCCGCATGGACTCTGAGGCGGTTAACCTTGCGCC TCTCCATCAGCGCCGAAGCGCGCAAGTCCATGGG ATGTAAGTACGCAGGCCTAGTCGTAGCCTAGCAT CGCCTATAGTGAGTCGTATTATCGGC (SEQ ID NO:83) |
| RHO-84 | DAF1-4560c-3 | Rho-10/11 | ATGCGCAGGACCCGGTGAGCCAGTCGGGTCGTG GCGTAACACACTCTGAGGCGGTTAACCTTGCGCC TCTCCATCAGCGCCGAAGCGCGAAGTGGTAGTGC TAGCACTTAGACAGGCCTAGTCGTAGCCTAGCAT CGCCTATAGTGAGTCGTATTATCGGC (SEQ ID NO:84) |
| RHO-85 | DAF1-4560c-4 | Rho-10/11 | ATGCGCAGGACCCGGTGAGCCAGAAAGCGAATG GGTTCACGAGTACTGAGGCGGTTAACCTTGCGCC TCTCCATCAGCGCCGAAGCGAGGTTGTCCTAGGT CTGGTGACGAACAGGCCTAGTCGTAGCCTAGCAT CGCCTATAGTGAGTCGTATTATCGGC (SEQ ID NO:85) |
| RHO-86 | DAF1-4560c-5 | Rho-10/11 | ATGCGCAGGACCCGGTGAGCCAGTGGCGGTAAC CGCACCCGGGTTCTGAGGCGGTTAACCTTGCGCC TCTCCATCAGCGCCGAAGCGAGGGGACGACCAA CAGGCTTGGCTTCAGGCCTAGTCGTAGCCTAGCA TCGCCTATAGTGAGTCGTATTATCGGC (SEQ ID NO:86) |
| RHO-87 | DAF1-4560c-6 | Rho-10/11 | ATGCGCAGGACCCGGTGAGCCACACGAATAGTG AGGGTCGAAATCCTGAGGCGGTTAGCCTTGCGCC TCTCCATCAGCGCCGAAGCGTGTCTACGATTAGG GACCTGTGGCGCAGGCCTAGTCGTAGCCTAGCA TCGCCTATAGTGAGTCGTATTATCGGC (SEQ ID NO:87) |
| RHO-88 | DAF1-4560c-7 | Rho-10/11 | ATGCGCAGGACCCGGTGAGCCATAACGGGTCCA CGATTGCTAAGACTGAGGCGGTTAACCTTGCGCC TCTCCATCAACGCCGAAGCGTGCAACTTGTTTTG GCTGACAAGCGCAGGCCTAGTCGTAGCCTAGCA TCGCCTATAGTGAGTCGTATTATCGGC (SEQ ID NO:88) |
| RHO-89 | DAF1-4560c-13 | Rho-10/11 | ATGCGCAGGACCCGGTGAGCCACGGCGAAAGTT ATCACGGGTAACCTGAGGCGGTTAACCTTGCGCC TCTCCATCAGCGCCGAAGCGTGGCAACGACTGAG TCTGTCGGCAdCAGGCCTAGTCGTAGCCTAGCAT CGCCTATAGTGAGTCGTATTATCGGC (SEQ ID NO:89)rr |
| RHO-90 | DAF1-4560c-14 | Rho-10/11 | ATGCGCAGGACCCGGTGAGCCAGCGGCAGGTG GCTCGCCGCAAGTCTGAGGCGGTTAACCTTGCGC CTCTCCATCAGCGCCGAAGCGCGACTTGATGCTC GGTTGTATCGAACAGGCCTAGTCGTAGCCTAGCA TCGCCTATAGTGAGTCGTATTATCGGC (SEQ ID NO:90) |
| RHO-91 | DAF1-4560c-17 | Rho-10/11 | ATGCGCAGGACCCGGTGAGCCAAACAGGGTTCT TTTGCTTGAACGCTGAGGCGGTTAACCTTGCGCC TCTCCATCATCGCCGAAGCGAGCGATCGTCTTTG ACTAGTATTCTCAGGCCTAGTCGTAGCCTAGCAT CGCCTATAGTGAGTCGTATTATCGGC (SEQ ID NO:91) |
| RHO-92 | DAF1-4560c-18 | Rho-10/11 | ATGCGCAGGACCCGGTGAGCCATTGGTCGCATG GCCTTCCTTATCCTGAGGCGGTTAACCTTGCGCC TCTCCATCAACGCCGAAGCGAGCCACAACATGGG TCGTGTTGGCGCAGGCCTAGTCGTAGCCTAGCAT CGCCTATAGTGAGTCGTATTATCGGC (SEQ ID NO:92) |
| RHO-93 | DAF2-30e-12 | Rho-1/3 | ATGCATGTCCGGACTGCCAGGTGCAAACTTCACG CTGAGGCGGTTAACCTTGCGCCTCTCCATCACCG CCGAAGCGGGGCGTGAAGCCTTATTCAGGTACA CCGGGCAGGGCTACCTATAGTGAGTCGTATTATC GGC (SEQ ID NO:93) |
| RHO-94 | DAF2-30e-17 | Rho-1/3 | ATGCATGTCCGGACTGCCAGGTGTACCCATTCGA CTGAGGCGGTTAACCTTGCGCCTCTCCATCATCG CCGAAGCGAGTCGTTGAGCTAGGCTCAGGTACA CCGGGCAGGGCTACCTATAGTGAGTCGTATTATC GGC (SEQ ID NO:94) |
| RHO-95 | DAF1-30c-3 | Rho-1/3 | ATGCATGTCCGGACTGCCAGGTGCAGATCGCACT CTGAGGCGGTTAACCTTGCGCCTCTCCATCATCG CCGAAGCGAAGAGTGCGCGAGTGATCAGGTACA CCGGGCAGGGCTACCTATAGTGAGTCGTATTATC GGC (SEQ ID NO:95) |
| RHO-96 | DAF1-30c-8 | Rho-1/3 | ATGCATGTCCGGACTGCCAGGTGCACCGTGACG ACTGAGGCGGTTAACCTTGCGCCTCTCCATCATC GCCGAAGCGAGGTCGTCGGAGAAATCCAGGTAC ACCGGGCAGGGCTACCTATAGTGAGTCGTATTAT CGGC (SEQ ID NO:96) |
| RHO-97 | DAF1-30c-10 | Rho-1/3 | ATGCATGTCCGGACTGCCACGGTGCACTTATGCT CTGAGGCGGTTAACATTGCGCCTCTCCATCATCG CCGAAGCGAGAGCATTGACACGTGTCAGGTACAC CGGGCAGGGCTACCTATAGTGAGTCGTATTATCG GC (SEQ ID NO:97) |
| RHO-98 | DAF1-4560e-1 | Rho-10/11 | ATGCGCAGGACCCGGTGAGCCAGGAACCTGAAC GTACCGCTTCTACATTTATAATGGGGCGGGGAAC CAGCGACATCCATCATCGCCGAAGCGTGGTTCCC AGGCCTAGTCGTAGCCTAGCATCGCCTATAGTGA GTCGTATTATCGGC (SEQ ID NO:98) |
| RHO-99 | DAF1-4560e-2 | Rho-10/11 | ATGCGCAGGACCCGGTGAGCCAGCTCGGGACCA ACACTCGCAAGACTGAGGCGGTTAACCTTGCGCC TCTCCATCAGCGCCGAAGCGCGTCAAGCAATCTC AACTAAATTCTCAGGCCTAGTCGTAGCCTAGCAT CGCCTATAGTGAGTCGTATTATCGGC (SEQ ID NO:99) |
| RHO-100 | DAF1-4560e-3 | Rho-10/11 | ATGCGCAGGACCCGGTGAGCCAGACGGGTCGCT TCATTCTTGTTCGTAAATCCTGAGGCGGTTAACCT TGCGCCTCTCCATCAGCGCCGAAGCGCGGACTAC TAGCAATTGGTGGTGCAGACTACTCAGGCCTAGT CGTAGCCTAGCATCGCCTATAGTGAGTCGTATTA TCGGC (SEQ ID NO:100) |
| RHO-101 | DAF1-4560e-4 | Rho-10/11 | ATGCGCAGGACCCGGTGAGCCAGGCGTAGGTGC GCGTTAGAGCACCTGAGGCGGTTAACCTTGCGCC TCTCCATCAACGCCGAAGCGAGGCCAGGCTAAGT GCTAAACTTGGCAGGCCTAGTCGTAGCCTAGCAT CGCCTATAGTGAGTCGTATTATCGGC (SEQ ID NO:101) |
| RHO-102 | DAF1-4560e-10 | Rho-10/11 | ATGCGCAGGACCCGGTGAGCCAGGGGCGCGTG GCTCCGTGGGTCGCTGAGGCGGTTAACCTTGCG CCTCTCCATCAGCGCCGAAGCGAGGATACGGTAG GTGCCGACCATCACAGGCCTAGTCGTAGCCTAG CATCGCCTATAGTGAGTCGTATTATCGGC (SEQ ID NO: 102) |

Selection of aptamers may be accomplished by any suitable method known in the art, including SELEX (Systemic Evolution of Ligands by Exponential enrichment). The SELEX scheme is described in detail in U.S. Pat. No. 5,270,163 to Gold et al.; Ellington and Szostak, “In Vitro Selection of RNA Molecules that Bind Specific Ligands,” Nature 346:818- 822 (1990); and Tuerk and Gold, “Systematic Evolution of Ligands by Exponential Enrichment: RNA Ligands to Bacteriophage T4 DNA Polymerase,” Science 249:505- 510 (1990), each of which is hereby incorporated by reference in their entirety. An established template-primer system (Bartel et al., “HIV-I Rev Regulation Involves Recognition of Non-Watson-Crick Base Pairs in Viral RNA,” Cell 67:529-536 (1991), which is hereby incorporated by reference in its entirety) can be adapted to produce oligonucleotides having a stretch of about 20-80 random bases sandwiched between constant regions.

### Targets

The target can be any biomaterial or small molecule including, without limitation, proteins, nucleic acids (RNA or DNA), lipids, oligosaccharides, carbohydrates, small molecules, hormones, cytokines, chemokines, cell signaling molecules, metabolites, organic molecules, and metal ions. Complexes of two or more molecules can be targets and include, without limitation, complexes have the following interactions: protein-protein, protein-cofactor, protein-inhibiting small molecules, protein-activating small molecules, protein-small molecules, protein-ion, protein-RNA, protein-DNA, DNA-, RNA-DNA, RNA-RNA, modified nucleic acids-DNA or RNA, aptamer-. In addition, targets that possess a mutation can be distinguished from wildtype forms of the target. In some embodiments, the target is associated with an analyte in a sample. Additional targets are described in detail below.

In some embodiments, the target is a nucleic acid and binds specifically to the target domain via hybridization (e.g., Watson-Crick base-pairing). The target domain of the aptamer includes a nucleotide sequence that is sufficiently complementary to its target so as to hybridize under appropriate conditions with the target nucleic acid in the sample. Nucleic acid targets can be any type of nucleic acid including, without limitation, DNA, RNA, LNA, PNA, UNA, genomic DNA, viral DNA, synthetic DNA, DNA with modified bases or backbone, mRNA, noncoding RNA, PIWI RNA, termini- associated RNA, promoter-associated RNA, tRNA, rRNA, microRNA, siRNA, post- transcriptionally modified RNA, synthetic RNA, RNA with modified bases or backbone, viral RNA, bacteria RNA, RNA aptamers, DNA aptamers, ribozymes, and DNAzymes.

In other embodiments, the target is a peptide of any length, including without limitation, phosphoproteins, lipid-modified proteins, nitrosylated proteins, sulfenated proteins, acylated proteins, methylated proteins, demethylated proteins, C— terminal amidated proteins, biotinylated proteins, formylated proteins, gamma-carboxylated proteins, glutamylated proteins, glycylated proteins, iodinated proteins, hydroxylated proteins, isoprenylated proteins, lipoylated proteins (including prenylation, myristoylation, famesylation, palmitoylation, or geranylation), proteins covalently linked to nucleotides such as ADP ribose (ADP-ribosylated) or flavin, oxidated proteins, proteins modified with phosphatidylinositol groups, proteins modified with pyroglutamate, sulfated proteins, selenoylated proteins, proteins covalently linked to another protein (including sumoylation, neddylation, ubiquitination, or ISGylation), citrullinated proteins, deamidated proteins, eliminylated proteins, disulfide bridged proteins, proteolytically cleaved proteins, proteins in which proline residues have been racemized, any peptides sequences that undergo the above mentioned modifications, and proteins which undergo one or more conformational changes. In addition, peptides having a mutation can be distinguished from wildtype forms.

Lipid targets include, without limitation, phospholipids, glycolipids, mono-, di-, tri-glycerides, sterols, fatty acyl lipids, glycerolipids, glycerophospholipids, sphingolipids, sterol lipids, prenol lipids, saccharolipids, polyketides, eicosanoids, prostaglandins, leukotrienes, thromboxanes, N-acyl ethanolamine lipids, cannabinoids, anandamides, terpenes, and lipopolysaccharides.

Small molecule targets include, without limitation, carbohydrates, monosaccharides, polysaccharides, galactose, fructose, glucose, amino acids, peptides, nucleic acids, nucleotides, nucleosides, cyclic nucleotides, polynucleotides, vitamins, drugs, inhibitors, single atom ions (such as magnesium, potassium, sodium, zinc, cobalt, lead, cadmium, etc.), multiple atom ions (such as phosphate), radicals (such as oxygen or hydrogen peroxide), and carbon-based gases (carbon dioxide, carbon monoxide, etc.).

Targets can also be whole cells or molecules expressed on the surface of whole cells. Exemplary cells include, without limitation, cancer cells, bacterial cells, or normal cells. Targets can also be viral particles.

### Linkers

The terms “linker” and “linker domain” are used interchangeably herein. The linker domain is positioned between the reporter domain and target domain. The linker may be about 16 nucleotides or less (for example 8 nucleotides per side), such as about 20 nucleotides or less, about 30 nucleotides or less, or about 40 nucleotides or less. In some embodiments, the linker domain is between about 2 and about 14 nucleotides, between about 4 and about 12 nucleotides, or between about 6 and about 10 nucleotides. The linker domain length may be determined by the needs of the targeting domain. In some embodiments, the linker is symmetrical with half of the linker one either side of the target domain. For example, a linker domain having 16 nucleotides would have two oligonucleotides of eight bases, one on either side flanking the target domain. In other embodiments, the linker domain is asymmetrical with different numbers of nucleotides on each side of the stem.

Linker domain sequences can be generated randomly or selected. Randomly generated linker sequences can be prepared and identified using a library. In some embodiments, libraries of various linker sequences of 4ⁿ can be prepared, where n is the number of nucleotides, unique sequences because the two sides of the linker domain may contain mismatches. These libraries are abbreviated as Nn libraries, where n is the number of nucleotides used in the construction of the library. If using Nn libraries of sufficient size, care should be taken to ensure necessary motifs are not duplicated between the linker domain and target domain.

One or more target domains in the biosensor are attached through one or more linker domains to the reporter domain. Together, the linker domain and the target domain form an additional stem on the reporter domain. Identifying suitable target domain comprising of a polynucleotide involves selecting polynucleotides that bind a particular target molecule with sufficiently high affinity (e.g., K_{d} ≤ 500 nM when not reduced by the linker domain) and specificity from a pool or library of nucleic acids containing a random region of varying or predetermined length.

One or more target domains may be attached to each linker domain. If more than one target domain is used, they may either bind to the same or different targets. Multiple target domains may work independently or together to effect an allosteric change in the biosensor.

The linker domain may or may not be positioned in such a way as to alter the binding of either reporter or target domain for their respective targets. The linker domain may be attached to either the reporter domain or the target domain in such a way to partially destabilize either binding pockets, but without destroying the binding pocket ability. For example, the linker may be coupled to a truncated target domain with a single base pair on the end of the conserved binding pocket in order to partially reduce the binding of the target domain to its target.

### Reporters

Reporters useful with biosensors of the present technology include reporters which omit a signal upon binding to the reporter domain of the aptamer. Non-limiting examples of reporters include luminescent molecules, chemiluminescent molecules, fluorochromes, fluorescent quenching agents, colored molecules, radioisotopes, mass reporters, biotin, avidin, streptavidin, protein A, protein G, antibodies or fragments thereof, polyhistidine, nickel and its ions, Flag tags, myc tags, heavy metals, enzymes, alkaline phosphatase, peroxidase, luciferase, and colorimetric substrates. In some embodiments, the reporter is a fluorochrome. Non-limiting examples of fluorochromes include fluorochromes having an excitation at a wavelength of 560 nm, 575 nm, and/or 580 nm and an emission wavelength of 596 nm, 600 nm, and/or 602 nm. For example, fluorochromes useful with biosensors of the present technology include rhodamine fluorochromes and variants thereof. Non-limiting examples include those described by Sunbul and Jaschke and Arora et al. In some embodiments, the rhodamine fluorochrome is a sulforhodamine. Example sulforhodamine compounds include di-nitro-sulforhodamine (SR-DN), such as the SR-DN illustrated in Formula I:

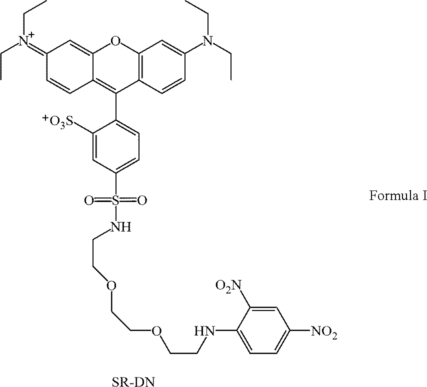

The reporter domain and the fluorophore (e.g., SR-DN) have a low dissociation constant, K_{d}, with the fluorophores (Sunbul and Jaschke). The K_{d} is at least about 0.5 µM, at least about 0.7 µM, at least about 1.0 µM, at least about 1.5 _{µ}M, or at least about 2.0 µM. The reporter domain has a fluorophore binding affinity of at least about 400 nM, about 300 nM, about 200 nM, about 100 nM, about 50 nM, about 40 nM, about 30 nM, about 20 nM, about 10 nM, about 5 nM, about 1 nM, or about 0.5 nM when the reporter domain is in a fluorophore binding conformation. When the reporter domain is bound to the reporter, biosensors of the present technology have a brightness of at least 7.000 M/cm, 8.000 M/cm, 9.000 M/cm, 10.000 M/cm, or 43.000 M/cm. The bound reporter has a fluorescent lifetime of at least 1 ns, or at least 2 ns, or at least 3 ns, or at least 4 ns or at least 5 ns, or at least 6 ns, or in the range of 1-6 ns, i.e. 1, or 2, or 3, or 4, or 5 or 6 ns.

While many of the embodiments described herein refer to sulforhodamine reporters and an SRB-2 aptamer as a backbone for the reporters having randomized regions, the present technology is not so limited, and biosensors are rather useful with different aptamers and different reporters. For example, different aptamer backbones can be used with the preset technology. In these examples, the aptamer backbones can include one or more randomized regions, such as those described herein or similar to those described herein.

### Additional Oligonucleotides

Additional oligonucleotides besides the aptamer and associated random regions described herein may also be included in the biosensor, at any position on the biosensor. In some embodiments, the additional oligonucleotides are linked to the aptamer and can be positioned on either end of the aptamer such that the aptamer retains function in the biosensor. Non-limiting examples of additional oligonucleotides include handles, barcodes, and/or promoters. Example sequences of handles include, but are not limited to:
Five prime (5′) handles (indicated in bold italics):
   GGTAGCCCTGCCCGGTGTACCTG (SEQ ID NO:8), and
   GGCGATGCTAGGCTACGACTAGGCCTG (SEQ ID NO:9).
Three prime (3′) handles (indicated in bold):
   ATGCATGTCCGGACTGCCA (SEQ ID NO:1), and
   ATGCGCAGGACCCGGTGAGCCA (SEQ ID NO:10).

Additional examples of oligonucleotides that may be included are promotor sequences. Any suitable promoter may be used including, but not limited to, a T7 promoter, which is used as the recognition/binding site for the transcriptase enzyme that causes RNA to be made from a dsDNA template during a transcription reaction. An example sequence of a T7 promoter (indicated in italics) includes, but is not limited to, GCCGATAATACGACTCACTATA (SEQ ID NO:2). In one embodiment, a T7 promoter may be linked to a reverse complement RNA-aptamer construct for production of the RNA-aptamer that is encoded by a double stranded DNA oligonucleotide.

In accordance with the embodiments described herein, RNA molecules that include an RNA aptamer sequence with randomized region(s), handles or primer sequences, and a T7 promoter sequence may be used to produce RNA aptamers.

Non-limiting examples of RNA molecules that include SRB-2 RNA aptamers having randomized regions of 30 nucleotides, handles (5′ in bold italics, 3′ in bold), and a promoter (italics) include:
N30-OSRB-2-UA-T7FR-RNA (RNA aptamer with randomized region replacing outside stem with T7 and handle additions and having the U-A base pair):

GCCGAUAAUACGACUCACUAUAGGUAGCCCUGCCCGGUGUACCU GNN NNNNNNNNNNNNNCUCGCUUCGGCGAUGAUGGAGAGGCGCA
AGGUU AACCGCCUCAGNNNNNNNNNNNNNNNUGGCAGUCCGGAC AUGCAU (SEQ ID NO:149);

N30-OSRB-2-CG-T7FR-RNA (RNA aptamer with randomized region replacing outside stem with T7 and handle additions and having the C-G base pair):

GCCGAUAAUACGACUCACUAUAGGUAGCCCUGCCCGGUGUACCU GNN NNNNNNNNNNNNNCCCGCUUCGGCGGUGAUGGAGAGGCGCA
AGGUU AACCGCCUCAGNNNNNNNNNNNNNNNUUGGCAGUCCGGA CAUGCAU (SEQ ID NO:150);

N30-ISRB-2-UA-T7FR-RNA (RNA aptamer with randomized region replacing inside stem with T7 and handle additions and having the U-A base pair):

GCCGAUAAUACGACUCACUAUAGGUAGCCCUGCCCGGUGUACCU GG GAACCUCGCUUCGGCGAUGAUGGAGNNNNNNNNNNNNNNNNN
NNNNN NNNNNNNNCAGGUUCCUGGCAGUCCGGACAUGCAU (SE
Q ID NO:152); and

N30-ISRB-2-CG-T7FR-RNA (RNA aptamer with randomized region replacing inside stem with T7 and handle additions and having the C-G base pair):

GCCGAUAAUACGACUCACUAUAGGUAGCCCUGCCCGGUGUACCU GG GAACCCCGCUUCGGCGGUGAUGGAGNNNNNNNNNNNNNNNNN
NNNN NNNNNNNNNCAGGUUCCUGGCAGUCCGGACAUGCAU (SE
Q ID NO:153).

Non-limiting examples of RNA molecules that include SRB-2 aptamers having randomized regions of 45 or 46 nucleotides, handles, and a promoter include
N46-OSRB-2-NN-T7FR-RNA (RNA aptamer with randomized region replacing outside stem with T7 and handle additions and having the N—N base pair):

GCCGAUAAUACGACUCACUAUAGGCGAUGCUAGGCUACGACUAGGCC UGNNNNNNNNNNNNNNNNNNNNNNNCNCGCUUCGGCGNUGAUGGAG
AGGCGCAAGGUUAACCGCCUCAGNNNNNNNNNNNNNNNNNNNNNNNU
GGCUCACCGGGUCCUGCGCAU (SEQ ID NO:155); and

N45-ISRB-2-NN-T7FR-RNA (RNA aptamer with randomized region replacing inside stem with T7 and handle additions and having the N—N base pair):

GCCGAUAAUACGACUCACUAUAGGCGAUGCUAGGCUACGACUAG GCC UGGGAACCNCGCUUCGGCGNUGAUGGANNNNNNNNNNNNNN
NNNNN NNNNNNNNNNNNNNNNNNNNNNNNNNCAGGUUCCUGGCU CACCGGG UCCUGCGCAU (SEQ ID NO:156).

Non-limiting examples of RNA molecules that include SRB-2 aptamers having randomized regions of 60 nucleotides, handles, and a promoter include
N60-OSRB-2-NN-T7FR-RNA (RNA aptamer with randomized region replacing outside stem with T7 and handle additions and having the N—N base pair):

GCCGAUAAUACGACUCACUAUAGGCGAUGCUAGGCUACGACUAG GCC UGNNNNNNNNNNNNNNNNNNNNNNNNNNNNNNCNCGCUUCG
GCGNU GAUGGAGAGGCGCAAGGUUAACCGCCUCAGNNNNNNNNN
NNNNNNNN NNNNNNNNNNNNNUGGCUCACCGGGUCCUGCGCAU
(SEQ ID NO:157); and

N60-ISRB-2-NN-T7FR-RNA (RNA aptamer with randomized region replacing inside stem with T7 and handle additions and having the N—N base pair)

GCCGAUAAUACGACUCACUAUAGGCGAUGCUAGGCUACGACUAG GCC UGGGAACCNCGCUUCGGCGNUGAUGGANNNNNNNNNNNNNN
NNNNN NNNNNNNNNNNNNNNNNNNNNNNNNNNNNNNNNNNNNNN
NNCAGGUU CCUGGCUCACCGGGUCCUGCGCAU (SEQ ID NO:
158).

Example sequences of primers include, but are not limited to,
F-Primer (Rho-8): GGTAGCCCTGCCCGGTGTACCTG (SEQ ID NO:8),
R-Primer (Rho-1): ATGCATGTCCGGACTGCCA (SEQ ID NO:1),
T7 Top (Rho-2): GCCGATAATACGACTCACTATA (SEQ ID NO:2),
FP w/ T7 (Rho-3): GCCGATAATACGACTCACTATAGGTAGCCCTGCCCGG TGTACCTG (SEQ ID NO:3),
F-Primer (Rho-9): GGCGATGCTAGGCTACGACTAGGCCTG (SEQ ID NO:9),
R-Primer (Rho-10): ATGCGCAGGACCCGGTGAGCCA (SEQ ID NO:10),
T7 Top (Rho-2) Ta = 38° C.: GCCGATAATACGACTCACTATA (SEQ ID NO:2), and
FP w/ T7 (Rho-11): GCCGATAATACGACTCACTATAGGCGATGCTAGGCTA CGACTAGGCCTG (SEQ ID NO:11).

Without intending to be limiting by the foregoing description, additional oligonucleotides can be useful as an aptamer, for sequencing the aptamer; generating the aptamer; and/or identifying the aptamer from a pool of aptamers. Table 1.1 includes non-limiting examples of such additional nucleotide sequences.

TABLE 1

Additional Nucleotide Sequences

| | | |
|---|---|---|
| ID | NAME | SEQUENCE |
| Rho-16 | N30-III-phase1 | TCGTCGGCAGCGTCAGATGTGTATAAGAGACAGGCATGCATGCATG CATGCGGTAGCCCTGCCCGGTGTACCTG (SEQ ID NO:16) |
| Rho-17 | N30-III-phase2 | TCGTCGGCAGCGTCAGATGTGTATAAGAGACAGTGCATGCATGCAT GCGGTAGCCCTGCCCGGTGTACCTG (SEQ ID NO:17) |
| Rho-18 | N30-III-phase3 | TCGTCGGCAGCGTCAGATGTGTATAAGAGACAGATGCATGCATGCG GTAGCCCTGCCCGGTGTACCTG (SEQ ID NO:18) |
| Rho-19 | N30-III-phase4 | TCGTCGGCAGCGTCAGATGTGTATAAGAGACAGCATGCATGCGGTA GCCCTGCCCGGTGTACCTG (SEQ ID NO:19) |
| Rho-20 | N30-III-reverse | GTCTCGTGGGCTCGGAGATGTGTATAAGAGACAGATGCATGTCCG GACTGCCA (SEQ ID NO:20) |
| Rho-21 | N4560-III-phase1 | TCGTCGGCAGCGTCAGATGTGTATAAGAGACAGGCATGCATGCATG CATGCGGCGATGCTAGGCTACGACTAGGCCTG (SEQ ID NO:21) |
| Rho-22 | N4560-III-phase2 | TCGTCGGCAGCGTCAGATGTGTATAAGAGACAGTGCATGCATGCAT GCGGCGATGCTAGGCTACGACTAGGCCTG (SEQ ID NO:22) |
| Rho-23 | N4560-III-phase3 | TCGTCGGCAGCGTCAGATGTGTATAAGAGACAGATGCATGCATGCG GCGATGCTAGGCTACGACTAGGCCTG (SEQ ID NO:23) |
| Rho-24 | N4560-III-phase4 | TCGTCGGCAGCGTCAGATGTGTATAAGAGACAGCATGCATGCGGC GATGCTAGGCTACGACTAGGCCTG (SEQ ID NO:24) |
| Rho-25 | N4560-lll-reverse | GTCTCGTGGGCTCGGAGATGTGTATAAGAGACAGATGCGCAGGAC CCGGTGAGCCA (SEQ ID NO:25) |
| Rho-26 | RNA-SRDN-DNA-RBD-4C | rGrGrArArCrCrUrCrGrCrUrUrCrGrGrCrGrArUrGrArUrGrGrArGrArUrUrUr ATCCAGAGTGACGCAGCATTTCATCGGGTCCAAAAGGGGCTGCTC GGGATTGCGGATATGGACACGTrArUrUrUrCrArGrGrUrUrCrC (SEQ ID NO:26) |
| Rho-27 | RNA-all-dU-4C | rGrGrArArCrCrUrCrGrCrUrUrCrGrGrCrGrArUrGrArUrGrGrArGrArUrUrUr A/ideoxyU/rCrCrArGrArG/ideoxyU/rGrArCrGrCrArGrCrA/ideoxyU//ideoxy U//ideoxyU/rCrA/ideoxyU/rCrGrGrG/ideoxyU/rCrCrArArArArGrGrGrGrC/i deoxyU/rGrC/ideoxyU/rCrGrGrGrA/ideoxyU//ideoxyU/rGrCrGrGrA/ideoxy U/rA/ideoxyU/rGrGrArCrArCrG/ideoxyU/rArUrUrUrCrArGrGrUrUrCrC (SEQ ID NO:27) |
| Rho-28 | RNA-all-4C | rGrGrArArCrCrUrCrGrCrUrUrCrGrGrCrGrArUrGrArUrGrGrArGrArUrUrUr ArUrCrCrArGrArGrUrGrArCrGrCrArGrCrArUrUrUrCrArUrCrGrGrGrUrC CrArArArArGrGrGrGrCrUrGrCrUrCrGrGrGrArUrUrGrCrGrGrArUrArUrGr GrArCrArCrGrUrArUrUrUrCrArGrGrUrUrCrC (SEQ ID NO:28) |
| Rho-29 | RNA-SRDN-DNA-RBD-1C | rGrGrArArCrCrCrCrGrCrUrUrCrGrGrCrGrGrUrGrArUrGrGrArGrArUrUrU CAGCACCGACCTTGTGCTTTGGGAGTGCTGGTCCAAGGGCGTTAAT GGACArArUrUrUrCrArGrGrUrUrCrC (SEQ ID NO:29) |
| Rho-30 | RNA-all-dU-1C | rGrGrArArCrCrCrCrGrCrUrUrCrGrGrCrGrGrUrGrArUrGrGrArGrArUrUrU CAGCACCGACC/ideoxyU//ideoxyU/G/ideoxyU/GC/ideoxyU//ideoxyU//id eoxyU/GGGAG/ideoxyU/GC/ideoxyU/GG/ideoxyU/CCAAGGGCG/ideoxy U//ideoxyU/AA/ideoxyU/GGACArArUrUrUrCrArGrGrUrUrCrC (SEQ ID NO:30) |
| Rho-31 | RNA-all-1C | rGrGrArArCrCrCrCrGrCrUrUrCrGrGrCrGrGrUrGrArUrGrGrArGrArUrUrUr CrArGrCrArCrCrGrArCrCrUrUrGrUGCrUrUrUrGrGrGrArGrUGrCrUrGrGr UrCrCrArArGrGrGrCrGrUrUrArArUrGrGrArCrArArUrUrUrCrArGrGrUrUr CrC (SEQ ID NO:31) |
| Rho-32 | GO-RBD-1C | /5FluorT/CAGCACCGACCTTGTGCTTTGGGAGTGCTGGTCCAAGGGC GTTAATGGACA (SEQ ID NO:32) |
| Rho-33 | GO-RBD-4C | /5FluorT/ATCCAGAGTGACGCAGCATTTCATCGGGTCCAAAAGGGGC TGCTCGGGATTGCGGATATGGACACGT (SEQ ID NO:33) |
| RHO-34 | SRDN handle control-CG | GGAACCTGAGGCGGTTAACCTTGCGCCTCTCCATCACCGCCGAAG CGGGGTTCCTATAGTGAGTCGTATTATCGGC (SEQ ID NO:34) |

### Methods of Preparing Biosensors

Biosensors of the present technology may be prepared using methods known to those of skill in the art using readily available reagents with no undue experimentation. For example, due to their small size, any known method may be used to synthesize the aptamers. By way of nonlimiting example, the aptamer may be produced using any method of synthetic oligonucleotide syntheses, preferably solid-state synthesis; PCR amplification; or produced in a cell by transforming a host cell with an expression vector comprising the aptamer operantly linked to a promoter capable of expressing the aptamer within the host cell. In some embodiments, the PCR amplification used to produce an aptamer described herein is any suitable method used to reverse transcribe an RNA aptamer (e.g., RT-PCR). In these embodiments, an RNA construct can be designed to include an aptamer sequence, a forward primer, a reverse primer, and a promoter that is used in the reverse transcription reaction to generate a reverse complement RNA (RC RNA)—i.e., a single stranded DNA (ssDNA) molecule-for producing RNA aptamers used in the biosensors discussed herein.

While the aptamer of the present technology can be synthesized from chemical precursor, they also can be prepared either in vitro or in vivo using recombinant templates or constructs, including transgenes, that encode the aptamers of the present technology. Whether using in vitro transcription or transgenes suitable for expression in vivo, these genetic constructs can be prepared using well known recombinant techniques. In some embodiments, genetic constructs useful with the present technology include a non-naturally occurring DNA molecule having a first region encoding an RNA aptamer molecule of the technology.

In some embodiments, the constructed DNA molecule encodes an RNA fusion product. Such a product is formed by joining together one piece of DNA encoding an RNA molecule of interest and a second piece of DNA encoding the reporter region that binds specifically to a reporter, and which may bind the RNA molecule of interest, the protein produced from the RNA of interest, or a downstream effect caused by the introduction of the RNA of interest into a host cell.

In some embodiments, the constructed DNA molecule encodes an aptamer of the disclosure, which is formed by joining together one piece of DNA encoding a target domain that is specific for a target ligand and a second piece of DNA encoding a receptor domain that binds specifically to a reporter, and a third piece of DNA encoding the linker domain.

In some embodiments, the aptamer may be made in a modular format though preparing an empty construct for preparation of specific domains of the aptamer. Such an empty construct includes a DNA sequence encoding one or more of the reporter, linker, and/or target domain(s), along with one or more regulatory sequences, and a restriction enzyme insertion site that can be used for subsequent insertion of a desired DNA molecule, which may encode the remaining domains. The restriction enzyme insertion site can include one or more enzymatic cleavage sites to facilitate insertion of virtually any DNA coding sequence as desired. The restriction enzyme insertion site is preferably located between the promoter sequence and the aptamer-encoding DNA sequence.

In some embodiments, the constructed DNA molecule encodes an aptamer or the disclosure, however, within the region encoding the aptamer, an intron is positioned therein. This spatially segregates the aptamer-encoding regions, whereby transcription in the absence of a proper spliceosome will not afford a functional aptamer molecule. In the presence of a proper spliceosome, excision of the intron from a transcript of the constructed DNA molecule affords the aptamer of the disclosure. This will allow the aptamer to bind to the reporter to induce a signal.

In an alternative embodiment, the sequences within the intron contribute to the reporter domain, whereby prior to splicing the RNA molecule is capable of exhibiting the signal when bound to the reporter. However, in the presence of a proper spliceosome, splicing of the RNA molecule destroys the reporter domain, thereby inhibiting the signal.

While the aptamers may be prepared with any nucleotide, RNA aptamers may be made from DNA molecules, such as expression vectors, in vitro or in vivo. Preparation of the DNA molecule can be carried out by well-known methods of DNA ligation. Once the expression vector or the aptamer of the present disclosure has been constructed, it can be incorporated into host cells as described herein. DNA molecules of the present technology optionally include a promoter operably coupled to the first region to control expression of the RNA aptamer. Depending on the application, it may be desirable to use strong promoters in order to obtain a high level of transcription. For instance, when used simply as a label high expression levels may be preferred, whereas to assess transcript behavior it may be desirable to obtain lower levels of expression that allow the cell to process the transcript.

Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the PR and PL promoters of coliphage lambda and others, including but not limited, to lacUV5, ompF, bla, Ipp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene. Initiation of transcription in mammalian cells requires a suitable promoter, which may include, without limitation, -globin, GAPDH, -actin, actin, Cstf2t, SV40, MMTV, metallothionine-1, adenovirus Ela, CMV immediate early, immunoglobulin heavy chain promoter and enhancer, and RSV-LTR. Termination of transcription in eukaryotic genes involves cleavage at a specific site in the RNA which may precede termination of transcription. Also, eukaryotic termination varies depending on the RNA polymerase that transcribes the gene. However, selection of suitable 3′ transcription termination regions is well known in the art and can be performed with routine skill.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Suitable promoters for use with the constructed DNA molecule of the present technology include, without limitation, a T7 promoter, a SUP4 tRNA promoter, an RPRI promoter, a GPD promoter, a GALI promoter, an hsp70 promoter, an Mtn promoter, a UAShs promoter, and functional fragments thereof. (Milligan et al., “Oligoribonucleotide Synthesis Using T7 RNA Polymerase and Synthetic DNA Templates,” Nucleic Acids Res. 15(21):8783-8798 (1987); Kurjan et al., Mutation at the Yeast SUP4 tRNAtyr Locus: DNA Sequence Changes in Mutants Lacking Suppressor Activity,” Cell 20:701-709 (1980); Lee et al., “Expression of RNase P RNA in Saccharomyces cerevisiae is Controlled by an Unusual RNA Polymerase III Promoter,” Proc. Natl. Acad. Sci. USA 88:6986-6990 (1991); Bitter et al., “Expression of Heterologous Genes in Saccharomyces cerevisiae from Vectors Utilizing the Glyceraldehyde-3-phosphate Dehydrogenase Gene Promoter,” Gene 32:263-274 (1984); Johnston and Davis, “Sequences that Regulate the Divergent GALI-GALIO Promoter in Saccharomyces cerevisiae,” Mal. Cell. Biol. 4:1440-1448 (1984); and Stuart et al., “A 12-Base-Pair Motif that is Repeated Several Times in Metallothionine Gene Promoters Confers Metal Regulation to a Heterologous Gene,” Proc. Natl. Acad. Sci. USA 81 :7318-7322 (1984)).

In some embodiments, DNA molecules include single-stranded DNA molecules having an aptamer nucleic acid sequence, a promoter nucleic acid sequence (italics), and at least one handle nucleic acid sequence (5′ in bold italics, 3′ in bold). In some embodiments, such DNA molecules, when transcribed, produce RNA aptamers that may be used in accordance with the embodiments described herein. Non-limiting examples of single-stranded DNA molecules useful with the present technology include:
N30-OSRB-2-UA-T7FR-DNA (Rho-4) (DNA aptamer with a N30 randomized region replacing outside stem with T7 and handle additions and having the U-A base pair):

ATGCATGTCCGGACTGCCANNNNNNNNNNNNNNNCTGAGGCGGT
TAA CCTTGCGCCTCTCCATCATCGCCGAAGCGAGNNNNNNNNNN
NNNNNCA GGTACACCGGGCAGGGCTACCTATAGTGAGTCGTATT
ATCGGC (SEQ ID NO:4),

N30-OSRB-2-CG-T7FR-DNA (Rho-5) (DNA aptamer with a N30 randomized region replacing outside stem with T7 and handle additions and having the C-G base pair):

ATGCATGTCCGGACTGCCANNNNNNNNNNNNNNNCTGAGGCGGT
TAA CCTTGCGCCTCTCCATCACCGCCGAAGCGGGNNNNNNNNNN
NNNNNC AGGTACACCGGGCAGGGCTACCTATAGTGAGTCGTATT
ATCGGC (SEQ ID NO:5),

N30-ISRB-2-UA-T7FR-DNA (Rho-6) (DNA aptamer with a N30 randomized region replacing inside stem with T7 and handle additions and having the U-A base pair):

ATGCATGTCCGGACTGCCAGGAACCTGNNNNNNNNNNNNNNNNN
NNN NNNNNNNNNNCTCCATCATCGCCGAAGCGAGGTTCCCAGGT ACACCG GGCAGGGCTACCTATAGTGAGTCGTATTATCGGC (SE
Q ID NO:6),

N30-ISRB-2-CG-T7FR-DNA (Rho-7) (DNA aptamer with a N30 randomized region replacing inside stem with T7 and handle additions and having the C-G base pair):

ATGCATGTCCGGACTGCCAGGAACCTGNNNNNNNNNNNNNNNNN
NNN NNNNNNNNNNCTCCATCACCGCCGAAGCGGGGTTCCCAGGT ACACCG GGCAGGGCTACCTATAGTGAGTCGTATTATCGGC (SE
Q ID NO:7),

N46-OSRB-2-NN-T7FR-DNA (Rho-12) (DNA aptamer with a N46 randomized region replacing outside stem with T7 and handle additions and having the N—N base pair):

ATGCGCAGGACCCGGTGAGCCANNNNNNNNNNNNNNNNNNNNNN
NCT GAGGCGGTTAACCTTGCGCCTCTCCATCANCGCCGAAGCGN
GNNNNNN NNNNNNNNNNNNNNNNNCAGGCCTAGTCGTAGCCTAG CATCGCCTATA GTGAGTCGTATTATCGGC (SEQ ID NO:12)
,

N45-ISRB-2-NN-T7FR-DNA (Rho-13) (DNA aptamer with a N45 randomized region replacing inside stem with T7 and handle additions and having the N—N base pair):

ATGCGCAGGACCCGGTGAGCCAGGAACCTGNNNNNNNNNNNNNN
NNN NNNNNNNNNNNNNNNNNNNNNNNNNNNNTCCATCANCGCCG
AAGCGN GGTTCCCAGGCCTAGTCGTAGCCTAGCATCGCCTATAG
TGAGTCGTAT TATCGGC (SEQ ID NO:13),

N60-OSRB-2-NN-T7FR-DNA (Rho-14) (DNA aptamer with a N60 randomized region replacing outside stem with T7 and handle additions and having the N—N base pair):

ATGCGCAGGACCCGGTGAGCCANNNNNNNNNNNNNNNNNNNNNN
NNN NNNNNCTGAGGCGGTTAACCTTGCGCCTCTCCATCANCGCC
GAAGCGN GNNNNNNNNNNNNNNNNNNNNNNNNNNNNNNCAGGCC TAGTCGTAGC CTAGCATCGCCTATAGTGAGTCGTATTATCGGC
(SEQ ID NO: 14), and

N60-ISRB-2-NN-T7FR-DNA (Rho-15) (DNA aptamer with a N60 randomized region replacing inside stem with T7 and handle additions and having the N—N base pair):

ATGCGCAGGACCCGGTGAGCCAGGAACCTGNNNNNNNNNNNNNN
NNN NNNNNNNNNNNNNNNNNNNNNNNNNNNNNNNNNNNNNNNNN
NNTCCA TCANCGCCGAAGCGNGGTTCCCAGGCCTAGTCGTAGCC TAGCATCGC CTATAGTGAGTCGTATTATCGGC (SEQ ID NO:
15).

Once the DNA molecule of the present technology has been constructed, it can be incorporated into cells using conventional recombinant DNA technology. Generally, this involves inserting the DNA molecule into an expression system to which the DNA molecule is heterologous (i.e., not normally present). The heterologous DNA molecule is inserted into the expression system or vector in proper sense orientation. The vector contains the necessary elements for their persistent existence inside cells and for the transcription of an RNA molecule that can be translated into the molecular complex of the present technology.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and transfection and replicated in cultures including prokaryotic organisms and eukaryotic cells grown in tissue culture.

Recombinant viruses can be generated by transfection of plasmids into cells infected with virus. Suitable vectors include, but are not limited to, the following viral vectors such as lambda vector system gtl 1, gt WES.tB, Charon 4, and plasmid vectors such as pBR322, pBR325, pACYCI 77, pACYC184, pUC8, pUC9, pUC18, pUC19, pLG339, pR290, pKC37, pKCIOI, SV 40, pBluescript II SK+/- or KS+/- (see “Stratagene Cloning Systems” Catalog (1993) from Stratagene, La Jolla, Calif), pQE, pIH821, pGEX, pET series (see Studier et al., “Use ofT7 RNA Polymerase to Direct Expression of Cloned Genes,” Gene Expression Technology, vol. 185 (1990), pIIIEx426 RPR, pIIIEx426 tRNA (see Good and Engelke, “Yeast Expression Vectors Using RNA Polymerase III Promoters,” Gene 151:209-214 (1994), p426GPD (see Mumberg et al., “Yeast Vectors for the Controlled Expression of Heterologous Proteins in Different Genetic Background,” Gene 156:119-122 (1995), p426GAL1 (see Mumberg et al., “Regulatable Promoters of Saccharomyces cerevisiae: Comparison of Transcriptional Activity and Their Use for Heterologous Expression,” Nucl. Acids Res. 22:5767-5768 (1994), pUAST (see Brand and Perrimon, “Targeted Gene Expression as a Means of Altering Cell Fates and Generating Dominant Phenotypes,” Development 118:401-415 (1993), and any derivatives thereof. Suitable vectors are continually being developed and identified.

A variety of host-vector systems may be utilized to express the DNA molecule. Primarily, the vector system should be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, adeno-associated virus, retroviral vectors, etc.); insect cell systems infected with virus (e.g., baculovirus); and plant cells infected by bacteria or transformed via particle bombardment (i.e., biolistics). The expression elements of these vectors vary in their strength and specificities. Depending upon the host-vector system utilized, any one of a number of suitable transcription elements can be used.

Once the constructed DNA molecule has been cloned into an expression system, it may be incorporated into a host cell. Such incorporation can be carried out by the various forms of transformation, depending upon the vector/host cell system such as transformation, transduction, conjugation, mobilization, or electroporation. The DNA sequences are cloned into the vector using standard cloning procedures in the art, as described by Maniatis et al., Molecular Cloning: A Laboratory Manual, Cold Springs Laboratory, Cold Springs Harbor, New York (1982). Suitable host cells include, but are not limited to, bacteria, yeast, mammalian cells, insect cells, plant cells, and the like. The host cell is preferably present either in a cell culture (ex vivo) or in a whole living organism (in vivo). Mammalian cells suitable for carrying out the present technology include, without limitation, COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, NS-I cells, embryonic stem cells, induced pluripotent stem cells, and primary cells recovered directly from a mammalian organism. With regard to primary cells recovered from a mammalian organism, these cells can optionally be reintroduced into the mammal from which they were harvested or into other animals.

As discussed above, aptamers are generally initially made using randomly generated sequences for both the linker domain and the target domain. Hence, they are initially made in pools of a mixture of different aptamers. Not all the aptamers in this initial pool will have the properties useful for biosensors of the present technology, namely a high affinity for both the reporter and the target ligand and undergo an allosteric shift which effects the signal emitted by the reporter of the biosensor in the presence of the target compared to the absence of the target ligand. Therefore, it is preferable for this initial pool to undergo enrichment selection to reduce the number of possible aptamers.

Any method of selection known in the art may be used to enrich for aptamers which undergo a shift in florescence due to an allosteric shift after a target binds to a target domain. For example, see U.S. Pat. App. No. 14/235,227 which uses selection in order to enrich for aptamers which bind to both the target and the reporter (e.g., fluorophore), and do not have cross binding to other fluorophores.

However, unlike in U.S. Pat. App. No. 14/235,227 it has been surprisingly found that using alternating rounds of “positive selection” and “negative selection” reduces the number of rounds of selection. As used herein, the term “positive selection” means an enrichment step where the aptamer will bind to the reporter (e.g., fluorophore) in the presence of the target. As used herein, the term “negative selection” means an enrichment step that removes aptamers that bind the fluorophore in the absence of the target.

To perform selection, the reporter (e.g., fluorophore) is bound to a solid substrate. This solid substrate may be any substrate known in the art, including, but not limited to, agarose beads, glass slides, or magnetic beads. The aptamers are then introduced to the bound fluorophores with either the target present or absent. For negative selection, the target is omitted from the process, but for positive selection the target is present. The mixture is incubated to allow time for allosteric binding, followed by washing. The elute resulting from negative selection contains aptamers which may bind the fluorophore only in the presence of the target or may not have an affinity for the fluorophore, therefore some of the aptamers in the elute may be suitable aptamers. The bound aptamers bind to the fluorophore without the target present and are therefore unsuitable as aptamers. The elute from the positive selection are the aptamers which will not bind to the fluorophore in the presence of the target and are therefore unsuitable for use in a biosensor of the present technology. The aptamers which bind the fluorophore in the presence of the target may be suitable as a biosensor, which can then be washed off the solid support. Therefore, the eluate of the negative selection and the bound aptamers in the positive selection may be suitable for use as a biosensor of the present technology.

Any combination of negative and positive selection may be performed. Preferably positive selection follows a round of negative selection to take advantage of the elute of a round of negative selection comprising of aptamers which may be suitable according to this disclosure. Preferably, about 10 or fewer, about 8 or fewer, about 6 or fewer, or about 4 or fewer total rounds of selection are performed. For example, a pool of potential aptamers would be put through a round of negative selection, followed by two alternating rounds of positive followed by negative selection. Following the rounds of enrichment selection, the resulting pool of aptamers may then be optionally sequenced and individual aptamers chosen.

Optionally, it has been surprisingly found that the number of rounds of selection may be minimized by comparing the changes in counts or fold changes between rounds of selection. For example, a pool of potential aptamers, A₀, may undergo a round of negative selection followed by a round of positive selection, pool A₁. Representative samples of A₀ and A₁ may then be sequenced and the counts of unique sequences (potential aptamers) normalized and compared. Aptamers may be selected that exhibit an increase in count from A₀ to A₁ or that have a high A₁:A₀ ratio as these may show allosteric fluorescence.

Another optional selection method would be to split a pool of potential aptamers, B₀, into two equal molar pools, B₀ₐ and B_{0b}. B₀ₐ may then undergo negative selection, B₁ₐ, and B_{0b} may undergo positive selection, B_{1b}. Representative samples of B₀, and the eluate of B₁ₐ, and B_{1b} may then be sequenced and the fold change of B₁ₐ and B_{1b} calculated based on B₀. Potential aptamers may then be selected based on the change in fold change, with some aptamers expected to dim in the presence of the target ligand while others would show allosteric fluorescence.

### Methods of Using Biosensors

Biosensors can be used as detection reagents to determine whether a target is present in a sample. Exemplary targets include pathogens, small molecules, solvents, and ions. The sample may be environmental, such as a water or soil sample, or be isolated from a subject, such as a human or animal blood or tissue sample. One skilled in the art would know how to obtain a sample for use with a biosensor of the present technology. Any of the exemplary targets can be detected individually, e.g., as a detection mechanism for a single target, or combined into a panel including more than two targets.

After the respective sample is obtained, a biosensor of the disclosure is introduced into the sample. The method optionally includes fixing a sample prior to introducing the biosensor, for example, to locate a position of a target within the sample, such as, but not limited to, subcellular structures, RNA, or cells, such as bacteria cells within an environmental sample. The sample, biosensor, and any additional reagents necessary for target binding and/or reporter function are combined and incubated for a period of time until the target binds the target domain. Upon binding of the reporter to the aptamer, a molecular complex between the biosensor and the target is formed.

Molecular complexes of the present disclosure can exist in vitro, in isolated form, or in vivo following introduction of the biosensor (or a genetic construction or expression system encoding the same) into a sample, such as, but not limited to, a host cell or isolated environment or subject sample. In some embodiments, the molecular complex includes at least the aptamer and the reporter bound to the reporter domain of the aptamer (collectively the biosensor), and one or more targets (bound specifically by the target domain(s)). These molecular complexes can exist in vitro, in isolated form or tethered to a substrate such as on an arrayed surface, or in vivo following introduction of the nucleic acid molecule (or a genetic construction or expression system encoding the same) into a host cell.

### Pathogens

Biosensors of the present disclosure are useful for detecting the presence of pathogens within a sample. Non-limiting examples of pathogens include bacterial, viral, prokaryotic, and fungal pathogens, or any non-naturally occurring biologic molecule in a host organism. Exemplary pathogens include, but are not limited to, adenovirus, coronavirus (e.g., HKU1, NL63, 229E, and OC43), human metapneumovirus, human rhinovirus/enterovirus, influenza (e.g., A, A/H1, A/H1-2009, A/H3, and B), parainfluenza (e.g., 1, 2, 3, and 4), respiratory syncytial virus, bordatella pertussis, chlamydophia penumoniae, SARS-CoV, SARS-CoV2 (e.g., COVID-19), MERS-CoV, UPEC, E. coli, klebsiella pneumoniae, proteus mirabilis, pseudomonas aeruginosa, staphylococcus saprophyticus, enterococcus faecalis, enterococcus faecim, clostridioides difficile, methicillin-resistant staphylococcus aureus, proteins synthesized by antibiotic resistant bacteria, West Nile virus, Zika virus, Ebola virus, salmonella, equine herpesvirus type I (EHV-1) and type IV (EHV-4), human immunodeficiency virus (HIV), hepatitis A, hepatitis B, hepatitis C, malaria, Dengue virus (DENV-1, -2, -3, -4, and -5), norovirus, rotavirus, astrovirus, Marburg virus, rabies, small pox, measles, and hantavirus. For example, the presence of SARS-CoV2 components, such as proteins (e.g., spike proteins), or portions thereof, can be detected within this sample. The detection of one or more SARS-CoV2 components in a sample can correlate to the presence of the SARS-CoV2 pathogen in the sample thereby detecting the presence of the SARS-CoV2 pathogen in the sample.

At least two or more of the foregoing exemplary pathogens can be combined into a panel, such as a respiratory panel or a urinary tract infection (UTI) panel. An exemplary respiratory panel includes adenovirus, coronavirus (e.g., HKU1, NL63, 229E, and OC43), human metapneumovirus, human rhinovirus/enterovirus, influenza (e.g., A, A/H1, A/H1-2009, A/H3, and B), parainfluenza (e.g., 1, 2, 3, and 4), respiratory syncytial virus, bordatella pertussis, chlamydophia penumoniae, SARS-CoV, SARS-CoV2 (e.g., COVID-19), MERS-CoV, or any combination thereof. An exemplary UTI panel includes, UPEC, E. coli, klebsiella pneumoniae, proteus mirabilis, pseudomonas aeruginosa, staphylococcus saprophyticus, enterococcus faecalis, enterococcus faecim, or any combination thereof.

### Small Molecules

Biosensors of the present disclosure are useful for detecting the presence of small molecules within a sample. Non-limiting examples of small molecules include toxins and pharmaceutical agents. Exemplary small molecules include, but are not limited to, cannabinoids (e.g., cannabidiol, cannabinol, and tetrahydrocannabinol), bisphenol A, fluoride, and benzene.

### Solvents

Biosensors of the present disclosure are useful for detecting the presence of solvents within a sample. Exemplary solvents include, but are not limited to, acetone, cyclohexane, acetic acid, ethanol, and benzene.

### Ions

Biosensors of the present disclosure are useful for detecting the presence of ions within a sample. Exemplary ions include, but are not limited to, potassium, chloride, sodium, lithium, magnesium, mercury, and lead.

### Detection of Target

Molecular complexes of the present disclosure can be exposed to an appropriate wavelength(s) of energy to activate the reporter (e.g., excitation wavelength) which emits the signal a different wavelength emitted (e.g., emission wavelength). The signal emitted by the reporter can be qualified or quantified based on a difference in brightness to determine if the target was present in the sample. For example, the difference in brightness of the biosensor in the sample to a control biosensor can be measured and determined. The change in brightness may either be an increase in brightness due to allosteric fluorescence or a dimming in brightness when compared to a control sample. For example, as discussed in the examples below, the emitted fluorescence signal for a SARS-CoV2 spike protein is lower than a fluorescence signal emitted by a control. As another example, the sample could be compared to a control reporter within the sample. A known quantity of the control reporter may be added across samples, allowing the comparison of signal from one sample to another.

In some embodiments, molecular complexes of the present disclosure can be identified, quantified, and monitored. Detection of molecular complex formation, through the fluorescent output of the fluorophore, a FRET partner (e.g., donor or acceptor), or a partner similar to a FRET partner can be used to detect complex formation in a cell-free sample (e.g., cell extracts, fractions of cell extracts, or cell lysates), histological or fixed samples, tissues or tissue extracts, bodily fluids, serum, blood and blood products, environmental samples, or in whole cells. Thus, detection and quantification can be carried out in vivo by fluorescence microscopy or the like, or detection and quantification can be carried in vitro on any of the above extracts or on a sample obtained via in vitro mixing of sample materials and reagents.

Regardless of the intended use, a suitable radiation source is used to illuminate the fluorophore after exposing the fluorophore and aptamer to one another. The radiation source can be used alone or with optical fibers and any optical waveguide to illuminate the sample. Suitable radiation sources include, without limitation, filtered, wide-spectrum light sources (e.g., tungsten, or xenon arc), laser light sources, such as gas lasers, solid state crystal lasers, semiconductor diode lasers (including multiple quantum well, distributed feedback, and vertical cavity surface emitting lasers), dye lasers, metallic vapor lasers, free electron lasers, and lasers using any other substance as a gain medium. Common gas lasers include Argon-ion, Krypton-ion, and mixed gas (e.g., Ar Kr) ion lasers, emitting at 455, 458, 466, 476, 488, 496, 502, 514, and 528 nm (Ar ion); and 406, 413, 415, 468, 476, 482, 520, 531, 568, 647, and 676 nm (Kr ion). Also included in gas lasers are Helium Neon lasers emitting at 543, 594, 612, and 633 mn. Typical output lines from solid state crystal lasers include 532 nm (doubled Nd:YAG) and 408/816 nm (doubled/primary from Ti:Sapphire). Typical output lines from semiconductor diode lasers are 635, 650, 670, and 780 mm. Infrared radiation sources can also be employed.

In certain embodiments, detection and quantification is carried out by a suitable commercially available plate (or microplate) reader that is capable of wavelength settings or filters that provide efficient excitation of the fluorophore and allow for detectable emission by the fluorophore (e.g., SR-DN). The emission spectra can then be used to compare brightness between samples containing the target vs. samples that do not contain the target. Excitation wavelengths and emission detection wavelengths vary depending on both the fluorophore and the nucleic acid aptamer molecule that are being employed. In certain embodiments, the plate reader filters are set to excitation wavelengths of approximately 575 nm ± 15 nm, and emission wavelengths of approximately 610 nm ± 15 nm. In certain embodiments, the plate reader filters are set to excitation wavelengths of approximately 575 nm ± 10 nm, and emission wavelengths of approximately 610 nm ± 10 nm. In certain embodiments, the plate reader filters are set to excitation wavelengths of approximately 575 nm ± 5 nm and emission detection wavelengths of approximately 610 nm ± 5 nm. In one embodiment, SR-DN has an excitation of 575 nm and an emission of 602 nm.

Detection of the emission spectra can be achieved using any suitable detection system. Exemplary detection systems include, without limitation, a cooled CCD camera, a cooled intensified CCD camera, a single-photon-counting detector (e.g., PMT or APD), dual-photon counting detector, spectrometer, fluorescence activated cell sorting (FACS) systems, fluorescence plate readers, fluorescence resonance energy transfer, and other methods that detect photons released upon fluorescence or other resonance energy transfer excitation of molecules.

In embodiments where the reporters are attached to substrates, such as a glass slide or in microarray format, it is desirable to reject any stray or background light in order to permit the detection of low intensity fluorescence signals. In one embodiment, a small sample volume (about 10 nl) is probed to obtain spatial discrimination by using an appropriate optical configuration, such as evanescent excitation or confocal imaging. Furthermore, background light can be minimized by the use of narrow-bandpass wavelength filters between the sample and the detector and by using opaque shielding to remove any ambient light from the measurement system.

In one embodiment, spatial discrimination of a molecular complex of the technology attached to a substrate in a direction normal to the interface of the substrate is obtained by evanescent wave excitation. This is illustrated in PCT Application Publ. No. WO/2010/096584 to Jaffrey and Paige. Evanescent wave excitation utilizes electromagnetic energy that propagates into the lower-index of refraction medium when an electromagnetic wave is totally internally reflected at the interface between higher and lower-refractive index materials. In this embodiment a collimated laser beam is incident on the substrate/solution interface at an angle greater than the critical angle for total internal reflection (TIR). This can be accomplished by directing light into a suitably shaped prism or an optical fiber. In the case of a prism, the substrate is optically coupled (via index-matching fluid) to the upper surface of the prism, such that TIR occurs at the substrate/solution interface on which the molecular complexes are immobilized. Using this method, excitation can be localized to within a few hundred nanometers of the substrate/solution interface, thus eliminating autofluorescence background from the bulk analyte solution, optics, or substrate. Target recognition is detected by a change in the fluorescent emission of the molecular complex, whether a change in intensity or polarization. Spatial discrimination in the plane of the interface (i.e., laterally) is achieved by the optical system.

In the embodiment described above, a TIRF evanescent wave excitation optical configuration is implemented using a detection system that includes a universal fluorescence microscope. Any fluorescent microscope compatible with TIRF can be employed. The TIRF excitation light or laser can be set at either an angle above the sample shining down on the sample, or at an angle through the objective shining up at the sample. Effective results can be obtained with immobilization of either the aptamer or the fluorophore using NHS-activated glass slides. The fluorophore containing a free amine (at the R1 position) can be used to react with the NHS-slide. RNA can be modified with a 5′ amine for NHS reactions by carrying out T7 synthesis in the presence of an amine modified GTP analog (commercially available).

In the several embodiments described above, the output of the detection system is preferably coupled to a processor for processing optical signals detected by the detector. The processor can be in the form of personal computer, which contains an input/output (I/O) card coupled through a data bus into the processor. CPU/processor receives and processes the digital output signal and can be coupled to a memory for storage of detected output signals. The memory can be a random-access memory (RAM) and/or read only memory (ROM), along with other conventional integrated circuits used on a single board computer as are well known to those of ordinary skill in the art. Alternatively, or in addition, the memory may include a floppy disk, a hard disk, CD ROM, or other computer readable medium which is read from and/or written to by a magnetic, optical, or other reading and/or writing system that is coupled to one or more processors. The memory can include instructions written in a software package (for image processing) for carrying out one or more aspects of the present technology as described herein.

### Kits

Kits of the present disclosure include, but are not limited to, kits having components useful for determining presence of a target in a sample. In some embodiments, the kit comprises the biosensor, such as the aptamer, the reporter (e.g., SR-DN), reference dye, buffers, solvents, additional nucleic acids, and/or instructions for use.

In accordance with some embodiments, a kit or system may include one or more plates containing a plurality of wells having a covalently or otherwise attached aptamer (or probe) for the detection of a target antigen (i.e., “assay plates”), According to certain embodiments, an aptamer (e.g., those disclosed herein) included as part of a biosensor can include an end modifier that allows the aptamer to be immobilized or tethered on a plate for analysis. In one embodiment, an aptamer may have an amino linker C12 modification at the 5′ end of the nucleotide sequence (“5AmMC12”) that allows for conjugation of the aptamer to a COOH-coated plate. In another embodiment, an aptamer may be modified to include a biotin modification at the 5′ end of the nucleotide sequence that allows for conjugation of the aptamer to a streptavidin-coated plate. In other embodiments, any suitable end modifier may be used to conjugate the aptamer to plate coated with a moiety to which the modifier can be covalently bound. In one embodiment, such a plate with one or more covalently bound or otherwise attached aptamers (or probes) thereto may include a microplate containing wells having a covalently attached aptamer (or probe) for the detection of CoV-2. In some embodiments, the microplate may include 6, 9, 12, 16, 24, 36, 96, 384, or 1536 wells.

According to the embodiments described herein, a kit or system may also include one or more of a buffer, a ready to use solution containing a reporter, and an excel (or other data analysis tool) template for analyzing the results of an assay performed using the kit or system. In some embodiments a kit or system includes one, two, three, four, five, six, seven, eight, nine, ten, eleven, twelve, thirteen, fourteen, fifteen, sixteen, seventeen, eighteen, nineteen, twenty, or more than twenty assay plates. In one embodiment, the kit or system includes five assay plates. In another embodiment, the kit or system includes ten assay plates. In some embodiments, the buffer included in a kit or system is a salt-based buffer. In some embodiments, the target antigen is a CoV-2 antigen. In certain embodiments, the aptamer(s) bound to the plate is one or more of the aptamers disclosed herein. In some embodiments, the reporter is a fluorescent molecule.

In one embodiment, a plate having one or more covalently bound aptamers thereto may be part of a system or kit used to detect a target when a sample is added to and interacts with the biosensor, as discussed in the Examples below. In another embodiment, the system or kit includes one or more of the components described in the Examples below.

The disclosure is further illustrated by the following example which should not be construed as limiting. The examples are illustrative only, and are not intended to limit, in any manner, any of the aspects described herein. The following examples do not in any way limit the technology.

Without further description, it is believed that one of ordinary skill in the art may, using the preceding description and the following illustrative examples, make and utilize the agents of the present disclosure and practice the claimed methods. The following working examples are provided to facilitate the practice of the present disclosure and are not to be construed as limiting in any way the remainder of the disclosure.

### EXAMPLES

### Example 1: SRB-2 Aptamer Backbones For Randomized Libraries (Prophetic)

This example describes the SRB-2 aptamer backbone useful for randomized libraries. The nucleotide sequence of SRB-2 is GGAACCCCGCUUCGGCG GUGAUGGAGAGGCGCAAGGUUAACCGCCUCAGGUUCC (SEQ ID NO:103). Randomized regions can replace or be inserted on a first stem (stem 2) of the SRB-2 aptamer at position “N_{Y}” (underlined) of the following sequences: GGAACCUN_{Y}AUGAUGGAGAGGCGCAAGGUUAACCGCCUCAGGUUCC (SEQ ID NO: 104), GGAACCCN_{Y}GUGAUGGAGAGGCGCAAGGUUAACCGCCUCAGGUUCC (SEQ ID NO: 105), and/or GGAACCNN_{Y}NUGAUGGAGAGGCGCAAGGUUAACCGCCUCAG GUUCC (SEQ ID NO: 106). FIG. 1 illustrates the SRB-2 aptamer sequence, a location of where the N_{Y} position is disposed on stem 2, as well as a reporter (e.g., SR-DN) having a Kd of 310 nM.

Randomized regions can also replace or be inserted on the outside stem of the SRB-2 aptamer at position “N_{Y}” (underlined) of the following sequences: N_{Y}CUCGCUUCGGCGAUGAUGGAGAGGCGCAAGGUUAACCGCCUCAGN_{Y} (SEQ ID NO: 116), N_{Y}CCCGCUUCGGCGGUGAUGGAGAGGCGCAAGGUUAACCGCCUCAGN_{Y} (SEQ ID NO: 117), and/or N_{Y}CNCGCUUCGGCGNUGAUGGAGAGGCGCAAGGUUAA CCGCCUCAGN_{Y} (SEQ ID NO: 118). FIG. 2 illustrates the SRB-2 aptamer sequence, a location of where the N_{Y} position is disposed on the outside stem, as well as a reporter (e.g., SR-DN) having a Kd of 310 nM.

Randomized regions can also replace or be inserted on the inside stem of the SRB-2 aptamer at position “N_{Y}” (underlined) of the following sequences: GGAACCUCGCUUCGGCGAUGAUGGAGN_{Y}CAGGUUCC (SEQ ID NO: 119), GGAACCCCGCUUCGGCGGUGAUGGAGN_{Y}CAGGUUCC (SEQ ID NO: 120), and/or GGAACCNCGCUUCGGCGNUGAUGGAGN_{Y}CAGGUUCC (SEQ ID NO:121), FIG. 2 illustrates the SRB-2 aptamer sequence, a location of where the N_{Y} position is disposed on the inside stem, as well as a reporter (e.g., SR-DN) having a Kd of 310 nM.

### Example 2: Replacing Stem 2 With Randomized Regions on SRB-2 Aptamer Backbones (Prophetic)

This example describes the SRB-2 aptamer backbone with a randomized region of 30 nucleotides, 45 nucleotides, or 60 nucleotides. Aptamers having randomized regions will be tested with SELEX and may be useful as a switch for THC with the reporter SR-DN. As shown in FIG. 1, the random region will replace or be inserted on stem 2.

The oligonucleotides that will be generated for Example 2 are as follows:
(1) RNA Components:
   (A) Randomized regions
      (30 nts) NNNNNNNNNNNNNNNNNNNNNNNNNNNNNN (SEQ ID NO: 162);
      (45 nts) NNNNNNNNNNNNNNNNNNNNNNNNNNNNNNNNN NNNNNNNNNNNN (SEQ ID NO:163); or
      (60 nts) NNNNNNNNNNNNNNNNNNNNNNNNNNNNNNN NNNNNNNNNNNNNNNNNNNNNNNNNNNNN (SEQ ID NO:165);
   (B) Five Prime Handle (Fh) (bold italics) GGTAGCCCTGCCCGGTGTACCTG (SEQ ID NO:8);
   (C) Three Prime Handle (Th) (bold) ATGCATGTCCGGACTGCCA (SEQ ID NO: 1); and
   (D) T7 Promoter Sequence (italics) GCCGATAATACGACTCACTATA (SEQ ID NO:2)
   (E) SRB2 (aptamer) sequence from Example 1
(2) RNA Domain Substitutions, no T7, no handles:
   (A) N30-2SRB-2 -UA (stem 2):
      GGAACCUNNNNNNNNNNNNNNNNNNNNNNNNNNNNNNAU GAUGGAGAGGCGCAAGGUUAACCGCCUCAGGUUCC (SEQ ID NO:107),
   (B) N30-2SRB-2-CG (stem 2):
      GGAACCCNNNNNNNNNNNNNNNNNNNNNNNNNNNNNNG UGAUGGAGAGGCGCAAGGUUAACCGCCUCAGGUUCC (SEQ ID NO:108),
   (C) N30-2SRB-2 —NN (stem 2):
      GGAACCNNNNNNNNNNNNNNNNNNNNNNNNNNNNNNNNU GAUGGAGAGGCGCAAGGUUAACCGCCUCAGGUUCC(SEQ ID NO:109)
   (D) N45-2SRB-2 -UA (stem 2):
      GGAACCUNNNNNNNNNNNNNNNNNNNNNNNNNNNNNNNN NNNNNNNNNNNNNAUGAUGGAGAGGCGCAAGGUUAACC GCCUCAGGUUCC (SEQ ID NO:110)
   (E) N45-2SRB-2-CG (stem 2):
      GGAACCCNNNNNNNNNNNNNNNNNNNNNNNNNNNNNNNN NNNNNNNNNNNNNGUGAUGGAGAGGCGCAAGGUUAACC GCCUCAGGUUCC (SEQ ID NO: 111)
   (F) N45-2SRB-2-NN (stem 2):
      GGAACCNNNNNNNNNNNNNNNNNNNNNNNNNNNNNNNNN NNNNNNNNNNNNNNUGAUGGAGAGGCGCAAGGUUAACC GCCUCAGGUUCC (SEQ ID NO:112)
   (G) N60-2SRB-2 -UA (stem 2):
      GGAACCUNNNNNNNNNNNNNNNNNNNNNNNNNNNNNNNN NNNNNNNNNNNNNNNNNNNNNNNNNNNNAUGAUGGAGA GGCGCAAGGUUAACCGCCUCAGGUUCC (SEQ ID NO:113)
   (H) N60-2SRB-2-CG (stem 2):
      GGAACCCNNNNNNNNNNNNNNNNNNNNNNNNNNNNNNNN NNNNNNNNNNNNNNNNNNNNNNNNNNNNGUGAUGGAGA GGCGCAAGGUUAACCGCCUCAGGUUCC (SEQ ID NO: 114)
   (I) N60-2SRB-2-NN (stem 2):
      GGAACCNNNNNNNNNNNNNNNNNNNNNNNNNNNNNNNNN NNNNNNNNNNNNNNNNNNNNNNNNNNNNNUGAUGGAGA GGCGCAAGGUUAACCGCCUCAGGUUCC (SEQ ID NO: 115)
(3) RNA Domain Substitutions with T7 (italics) and Handle Additions (5′ in bold italics, 3′ in bold):
   (A) N30-2SRB-2-UA-T7FR-RNA (outside):
      GCCGAUAAUACGACUCACUAUAGGUAGCCCUGCCCGGU GUACCUGGGAACCUNNNNNNNNNNNNNNNNNNNNNNNN NNNNNNAUGAUGGAGAGGCGCAAGGUUAACCGCCUCAG GUUCCUGGCAGUCCGGACAUGCAU (SEQ ID NO: 140)
   (B) N30-2SRB-2-CG-T7FR-RNA (outside):
      GCCGAUAAUACGACUCACUAUAGGUAGCCCUGCCCGGU GUACCUGGGAACCCNNNNNNNNNNNNNNNNNNNNNNNN NNNNNNGUGAUGGAGAGGCGCAAGGUUAACCGCCUCAG GUUCCUGGCAGUCCGGACAUGCAU (SEQ ID NO: 141)
   (C) N30-2SRB-2-NN-T7FR-RNA (outside):
      GCCGAUAAUACGACUCACUAUAGGUAGCCCUGCCCGGU GUACCUGGGAACCNNNNNNNNNNNNNNNNNNNNNNNNN NNNNNNNUGAUGGAGAGGCGCAAGGUUAACCGCCUCAG GUUCCUGGCAGUCCGGACAUGCAU (SEQ ID NO: 142)
   (D) N45-2SRB-2-UA-T7FR-RNA (outside):
      GCCGAUAAUACGACUCACUAUAGGUAGCCCUGCCCGGU GUACCUGGGAACCUNNNNNNNNNNNNNNNNNNNNNNNN NNNNNNNNNNNNNNNNNNNNNAUGAUGGAGAGGCGCAA GGUUAACCGCCUCAGGUUCCUGGCAGUCCGGACAUGCA U (SEQ ID NO:143)
   (E) N45-2SRB-2-CG-T7FR-RNA (outside):
      GCCGAUAAUACGACUCACUAUAGGUAGCCCUGCCCGGU GUACCUGGGAACCCNNNNNNNNNNNNNNNNNNNNNNNN NNNNNNNNNNNNNNNNNNNNNGUGAUGGAGAGGCGCAA GGUUAACCGCCUCAGGUUCCUGGCAGUCCGGACAUGCA U (SEQ ID NO:144)
   (F) N45-2SRB-2-NN-T7FR-RNA (outside):
      GCCGAUAAUACGACUCACUAUAGGUAGCCCUGCCCGGU GUACCUGGGAACCNNNNNNNNNNNNNNNNNNNNNNNNN NNNNNNNNNNNNNNNNNNNNNNUGAUGGAGAGGCGCAA GGUUAACCGCCUCAGGUUCCUGGCAGUCCGGACAUGCA U (SEQ ID NO:145)
   (G) N60-2SRB-2-UA-T7FR-RNA (outside):
      GCCGAUAAUACGACUCACUAUAGGUAGCCCUGCCCGGU GUACCUGGGAACCUNNNNNNNNNNNNNNNNNNNNNNNN NNNNNNNNNNNNNNNNNNNNNNNNNNNNNNNNNNNNAUG AUGGAGAGGCGCAAGGUUAACCGCCUCAGGUUCCUGGC AGUCCGGACAUGCAU (SEQ ID NO: 146)
   (H) N60-2SRB-2-CG-T7FR-RNA (outside):
      GCCGAUAAUACGACUCACUAUAGGUAGCCCUGCCCGGU GUACCUGGGAACCCNNNNNNNNNNNNNNNNNNNNNNNN NNNNNNNNNNNNNNNNNNNNNNNNNNNNNNNNNNNNGU GAUGGAGAGGCGCAAGGUUAACCGCCUCAGGUUCCUGG CAGUCCGGACAUGCAU (SEQ ID NO: 147)
   (I) N60-2SRB-2-NN-T7FR-RNA (outside):
      GCCGAUAAUACGACUCACUAUAGGUAGCCCUGCCCGGU GUACCUGGGAACCNNNNNNNNNNNNNNNNNNNNNNNNN NNNNNNNNNNNNNNNNNNNNNNNNNNNNNNNNNNNNNU GAUGGAGAGGCGCAAGGUUAACCGCCUCAGGUUCCUGG CAGUCCGGACAUGCAU (SEQ ID NO: 148)

### Example 3: Thirty Nucleotide Randomized Region on SRB-2 Aptamer Backbones (Prophetic)

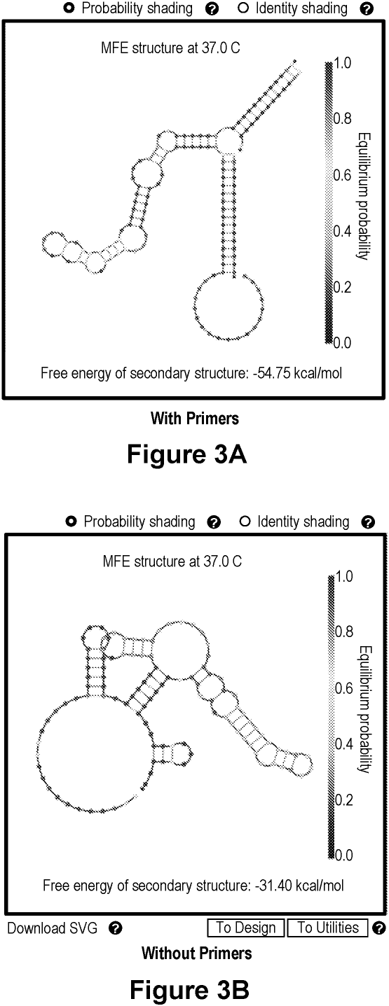

This example describes the SRB-2 aptamer backbone with a randomized 30 nucleotide (N=30) region. Aptamers having randomized regions will be tested with SELEX and may be useful as a switch for THC with the reporter SR-DN. As shown in FIG. 2, the random region will replace or be inserted on the outside stem (circle near bottom) or the inside stem (circle near SR-DN). Predictive thermograms for N30-SRB-2 libraries with and without primers are shown in FIGS. 3A and 3B, respectively.

The oligonucleotides that will be generated for Example 3 are as follows:
(1) RNA Components:
   (A) Randomized region (30 nts) NNNNNNNNNNNNNNNNNNNNNNNNNNNNNN(SEQ ID NO:162);
   (B) Five Prime Handle (Fh) (bold italics) GGTAGCCCTGCCCGGTGTACCTG (SEQ ID NO:8);
   (C) Three Prime Handle (Th) (bold) ATGCATGTCCGGACTGCCA (SEQ ID NO:1);
   (D) T7 Promoter Sequence (italics) GCCGATAATACGACTCACTATA (SEQ ID NO:2)
   (E) SRB2 (aptamer) sequence from Example 1
(2) RNA Domain Substitutions, no T7, no handles:
   (A) N30-OSRB-2 -UA (outside):
      NNNNNNNNNNNNNNNCUCGCUUCGGCGAUGAUGGAGAG GCGCAAGGUUAACCGCCUCAGNNNNNNNNNNNNNNN (SEQ ID NO:122)
   (B) N30-OSRB-2-CG (outside):
      NNNNNNNNNNNNNNNCCCGCUUCGGCGGUGAUGGAGAG GCGCAAGGUUAACCGCCUCAGNNNNNNNNNNNNNNN (SEQ ID NO:123)
   (C) N30-OSRB-2 —NN (outside):
      NNNNNNNNNNNNNNNCNCGCUUCGGCGNUGAUGGAGAG GCGCAAGGUUAACCGCCUCAGNNNNNNNNNNNNNNN (SEQ ID NO:124)
   (D) N30-ISRB-2 -UA (inside):
      GGAACCUCGCUUCGGCGAUGAUGGAGNNNNNNNNNNNN NNNNNNNNNNNNNNNNNNCAGGUUCC (SEQ ID NO:125)
   (E) N30-ISRB-2-CG (inside):
      GGAACCCCGCUUCGGCGGUGAUGGAGNNNNNNNNNNNN NNNNNNNNNNNNNNNNNNCAGGUUCC (SEQ ID NO:126)
   (F) N30-ISRB-2-NN (inside):
      GGAACCNCGCUUCGGCGNUGAUGGAGNNNNNNNNNNNN NNNNNNNNNNNNNNNNNNCAGGUUCC (SEQ ID NO:127)
(3) RNA Domain Substitutions with T7 (italics) and Handle Additions (5′ in bold italics, 3′ in bold):
   (A) N30-OSRB-2-UA-T7FR-RNA (outside):
      GCCGAUAAUACGACUCACUAUAGGUAGCCCUGCCCGGU GUACCUGNNNNNNNNNNNNNNNCUCGCUUCGGCGAUGA UGGAGAGGCGCAAGGUUAACCGCCUCAGNNNNNNNNNN NNNNNUGGCAGUCCGGACAUGCAU (SEQ ID NO: 149)
   (B) N30-OSRB-2-CG-T7FR-RNA (outside):
      GCCGAUAAUACGACUCACUAUAGGUAGCCCUGCCCGGU GUACCUGNNNNNNNNNNNNNNNCCCGCUUCGGCGGUGA UGGAGAGGCGCAAGGUUAACCGCCUCAGNNNNNNNNNN NNNNNUUGGCAGUCCGGACAUGCAU (SEQ ID NO: 150)
   (C) N30-OSRB-2-NN-T7FR-RNA (outside):
      GCCGAUAAUACGACUCACUAUAGGUAGCCCUGCCCGGU GUACCUGNNNNNNNNNNNNNNNCNCGCUUCGGCGNUGA UGGAGAGGCGCAAGGUUAACCGCCUCAGNNNNNNNNNN NNNNNUUGGCAGUCCGGACAUGCAU (SEQ ID NO:151)
   (D) N30-ISRB-2-UA-T7FR-RNA (inside):
      GCCGAUAAUACGACUCACUAUAGGUAGCCCUGCCCGGU GUACCUGGGAACCUCGCUUCGGCGAUGAUGGAGNNNNN NNNNNNNNNNNNNNNNNNNNNNNNNCAGGUUCCUGGCA GUCCGGACAUGCAU (SEQ ID NO: 152)
   (E) N30-ISRB-2-CG-T7FR-RNA (inside):
      GCCGAUAAUACGACUCACUAUAGGUAGCCCUGCCCGGU GUACCUGGGAACCCCGCUUCGGCGGUGAUGGAGNNNNN NNNNNNNNNNNNNNNNNNNNNNNNNCAGGUUCCUGGCA GUCCGGACAUGCAU (SEQ ID NO: 153)
   (F) N30-ISRB-2-NN-T7FR-RNA (inside):
      GCCGAUAAUACGACUCACUAUAGGUAGCCCUGCCCGGU GUACCUGGGAACCNCGCUUCGGCGNUGAUGGAGNNNNN NNNNNNNNNNNNNNNNNNNNNNNNNCAGGUUCCUGGCA GUCCGGACAUGCAU (SEQ ID NO: 154)
(4) ssDNA (RC RNA) with T7 Promoter and handles:
   (A) N30-OSRB-2-UA-T7FR-DNA (outside):
      ATGCATGTCCGGACTGCCANNNNNNNNNNNNNNNCTGAG GCGGTTAACCTTGCGCCTCTCCATCATCGCCGAAGCGAGN NNNNNNNNNNNNNNCAGGTACACCGGGCAGGGCTACCT ATAGTGAGTCGTATTATCGGC (SEQ ID NO:4)
   (B) N30-OSRB-2-CG-T7FR-DNA (outside):
      ATGCATGTCCGGACTGCCANNNNNNNNNNNNNNNCTGAG GCGGTTAACCTTGCGCCTCTCCATCACCGCCGAAGCGGG NNNNNNNNNNNNNNNCAGGTACACCGGGCAGGGCTACC TATAGTGAGTCGTATTATCGGC (SEQ ID NO:5)
   (C) N30-OSRB-2-NN-T7FR-DNA (outside):
      ATGCATGTCCGGACTGCCANNNNNNNNNNNNNNNCTGAG GCGGTTAACCTTGCGCCTCTCCATCANCGCCGAAGCGNG NNNNNNNNNNNNNNNCAGGTACACCGGGCAGGGCTACC TATAGTGAGTCGTATTATCGGC (SEQ ID NO:41)
   (D) N30-ISRB-2-UA-T7FR-DNA (inside):
      ATGCATGTCCGGACTGCCAGGAACCTGNNNNNNNNNNNN NNNNNNNNNNNNNNNNNNCTCCATCATCGCCGAAGCGAG GTTCCCAGGTACACCGGGCAGGGCTACCTATAGTGAGTC GTATTATCGGC (SEQ ID NO:6)
   (E) N30-ISRB-2-CG-T7FR-DNA (inside):
      ATGCATGTCCGGACTGCCAGGAACCTGNNNNNNNNNNNN NNNNNNNNNNNNNNNNNNCTCCATCACCGCCGAAGCGGG GTTCCCAGGTACACCGGGCAGGGCTACCTATAGTGAGTC GTATTATCGGC(SEQ ID NO:7)
   (F) N30-ISRB-2-NN-T7FR-DNA (inside):
      ATGCATGTCCGGACTGCCAGGAACCTGNNNNNNNNNNNN NNNNNNNNNNNNNNNNNNCTCCATCANCGCCGAAGCGNG GTTCCCAGGTACACCGGGCAGGGCTACCTATAGTGAGTC GTATTATCGGC (SEQ ID NO:42)
(5) Primers:
   (A) Taq Ta = 58 _{°}C: Phusion Ta = 67 ◦C
      (i) F-Primer (Rho-8): GGTAGCCCTGCCCGGTGTACCTG (SEQ ID NO:8)
      (ii) R-Primer (Rho-1): ATGCATGTCCGGACTGCCA (SEQ ID NO:1)
   (B) T7 Top (Rho-2) Ta = 38 ◦C: GCCGATAATACGACTCACTATA (SEQ ID NO:2)
   (C) Taq Ta = 57 ◦C: Kappa HiFi Ta = 67 ◦C
      (i) FP w/ T7 (Rho-3):
         GCCGATAATACGACTCACTATAGGTAGCCCTGCCC GGTGTACCTG (SEQ ID NO:3)
(6) Libraries:
   GGCAACGATCGCGTCTCCTCTCCGCACCTTCGAATAAGTACAAG TTGTACGCATCTTTTCATCCCTTCCGCGATCGTTGCCTATAGTGA GTCGTATTATC (SEQ ID NO: 159)
   (A) Rho-4
      (i) N30-OSRB-2-UA-T7FR-DNA (outside):
         ATGCATGTCCGGACTGCCANNNNNNNNNNNNNNNC TGAGGCGGTTAACCTTGCGCCTCTCCATCATCGCC GAAGCGAGNNNNNNNNNNNNNNNCAGGTACACCG GGCAGGGCTACCTATAGTGAGTCGTATTATCGGC (SEQ ID NO:4)
   (B) Rho-5
      (i) N30-OSRB-2-CG-T7FR-DNA (outside):
         ATGCATGTCCGGACTGCCANNNNNNNNNNNNNNNC TGGGCGGTTCCTTGCGCCTCTCCTCCCGCCGGCGG GNNNNNNNNNNNNNNNCAGGTACACCGGGCAGGG CTACCTATAGTGAGTCGTATTATCGGC (SEQ ID NO:5)
   (C) Rho 41
      (i) N30-OSRB-2-NN-T7FR-DNA (outside):
         ATGCATGTCCGGACTGCCANNNNNNNNNNNNNNNC TGAGGCGGTTAACCTTGCGCCTCTCCATCANCGCC GAAGCGNGNNNNNNNNNNNNNNNCAGGTACACCG GGCAGGGCTACCTATAGTGAGTCGTATTATCGGC (SEQ ID NO:41)
   (D) Rho-6
      (i) N30-ISRB-2-UA-T7FR-DNA (inside):
         ATGCATGTCCGGACTGCCAGGAACCTGNNNNNNNN NNNNNNNNNNNNNNNNNNNNNNCTCCATCATCGCC GAAGCGAGGTTCCCAGGTACACCGGGCAGGGCTA CCTATAGTGAGTCGTATTATCGGC (SEQ ID NO:6)
   (E) Rho-7
      (i) N30-ISRB-2-CG-T7FR-DNA (inside):
         ATGCATGTCCGGACTGCCAGGAACCTGNNNNNNNN NNNNNNNNNNNNNNNNNNNNNNCTCCATCACCGCC GAAGCGGGGTTCCCAGGTACACCGGGCAGGGCTA CCTATAGTGAGTCGTATTATCGGC (SEQ ID NO:7)
   (F) Rho-42
      (i) N30-ISRB-2-NN-T7FR-DNA (inside):
         ATGCATGTCCGGACTGCCAGGAACCTGNNNNNNNN NNNNNNNNNNNNNNNNNNNNNNCTCCATCANCGCC GAAGCGNGGTTCCCAGGTACACCGGGCAGGGCTA CCTATAGTGAGTCGTATTATCGGC (SEQ ID NO:42)

### Example 4: Forty-Five or Forty-Six Nucleotide Randomized Region on SRB-2 Aptamer Backbones (Prophetic)

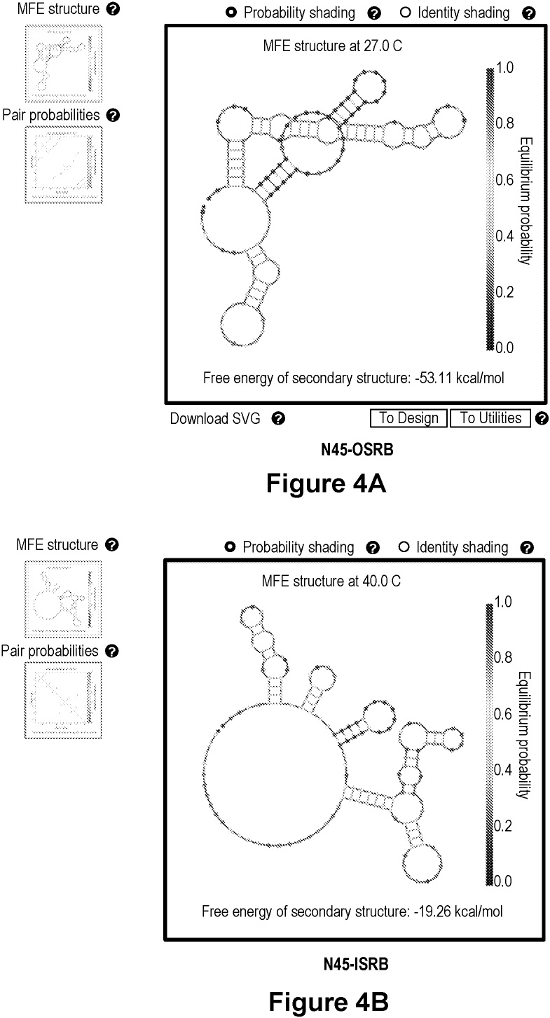

This example describes the SRB-2 aptamer backbone with a randomized 45 nucleotide (N=45) or 46 nucleotide (N=46) region for binding to larger targets, such as proteins (e.g., spike proteins expressed by a virus). Primers of Example 4 have higher melting peak temperatures compared to the Rho primers of Example 3. Aptamers having randomized regions will be tested with SELEX and may be useful as a switch for THC with the reporter SR-DN. As shown in FIG. 2, the random region will replace or be inserted on the outside stem (circle near bottom) or the inside stem (circle near SR-DN). Predictive thermograms for N45-SRB-2 libraries using the OSRB format and the ISRB format are shown in FIGS. 4A and 4B, respectively.

The oligonucleotides that will be generated for Example 4 are as follows:
(1) RNA Components:
   (A) Randomized region (45 nts or 46 nts) NNNNNNNNNNNNNNNNNNNNNNNNNNNNNNNNNNNNNNN NNNNNN (SEQ ID NO:163) (N45); or NNNNNNNN NNNNNNNNNNNNNNN (SEQ ID NO:161) and NNNNN NNNNNNNNNNNNNNNNNN (SEQ ID NO:161) (together, N46);
   (B) Five Prime Handle (Fh) (bold italics) GGCGATGCTAGGCTACGACTAGGCCTG (SEQ ID NO:9);
   (C) Three Prime Handle (Th) (bold) ATGCGCAGGACCCGGTG AGCCA (SEQ ID NO:10);
   (D) T7 Promoter Sequence (italics) GCCGATAATACGACTCAC TATA (SEQ ID NO:2);
   (E) SRB2 (aptamer) sequence from Example 1
(2) RNA Domain Substitutions, no T7, no handles:
   (A) N45-OSRB-2 -UA: (outside) NNNNNNNNNNNNNNNNNNNNNNNCUCGCUUCGGCGAUG AUGGAGAGGCGCAAGGUUAACCGCCUCAGNNNNNNNNN NNNNNNNNNNNNNN (SEQ ID NO:128)
   (B) N45-OSRB-2-CG: (outside) NNNNNNNNNNNNNNNNNNNNNNNCCCGCUUCGGCGGUG AUGGAGAGGCGCAAGGUUAACCGCCUCAGNNNNNNNNN NNNNNNNNNNNNNN (SEQ ID NO:129)
   (C) N45-ISRB-2 -UA: (inside) GGAACCUCGCUUCGGCGAUGAUGGAGNNNNNNNNNNNN NNNNNNNNNNNNNNNNNNNNNNNNNNNNNNNNNCAGGU UCC (SEQ ID NO:131)
   (D) N45-ISRB-2-CG: (inside) GGAACCCCGCUUCGGCGGUGAUGGAGNNNNNNNNNNNN NNNNNNNNNNNNNNNNNNNNNNNNNNNNNNNNNCAGGU UCC (SEQ ID NO:132)
(3) RNA Domain Substitutions with T7 and Handle Additions:
   (A) N46-OSRB-2-NN-T7FR-RNA (outside):
      GCCGAUAAUACGACUCACUAUAGGCGAUGCUAGGCUAC GACUAGGCCUGNNNNNNNNNNNNNNNNNNNNNNNCNCG CUUCGGCGNUGAUGGAGAGGCGCAAGGUUAACCGCCUC AGNNNNNNNNNNNNNNNNNNNNNNNUGGCUCACCGGGU CCUGCGCAU (SEQ ID NO: 155)
   (B) N45-ISRB-2-NN-T7FR-RNA (inside):
      GCCGAUAAUACGACUCACUAUAGGCGAUGCUAGGCUAC GACUAGGCCUGGGAACCNCGCUUCGGCGNUGAUGGANN NNNNNNNNNNNNNNNNNNNNNNNNNNNNNNNNNNNNNNN NNNNCAGGUUCCUGGCUCACCGGGUCCUGCGCAU (SEQ ID NO:156)
(4) ssDNA (RC RNA) with T7 Promoter and handles:
   (A) N46-OSRB-2-NN-T7FR-DNA (outside):
      ATGCGCAGGACCCGGTGAGCCANNNNNNNNNNNNNNNN NNNNNNNCTGAGGCGGTTAACCTTGCGCCTCTCCATCANC GCCGAAGCGNGNNNNNNNNNNNNNNNNNNNNNNNCAGG CCTAGTCGTAGCCTAGCATCGCCTATAGTGAGTCGTATTA TCGGC (SEQ ID NO:12)
   (B) N45-ISRB-2-NN-T7FR-DNA (inside):
      ATGCGCAGGACCCGGTGAGCCAGGAACCTGNNNNNNNN NNNNNNNNNNNNNNNNNNNNNNNNNNNNNNNNNNNNNTC CATCANCGCCGAAGCGNGGTTCCCAGGCCTAGTCGTAGC CTAGCATCGCCTATAGTGAGTCGTATTATCGGC (SEQ ID NO:13)
(5) Primers:
   (A) Taq Ta = 64 ◦C: Phusion Ta = 72 ◦C
      (i) F-Primer (Rho-9) (bold italics):
         GGCGATGCTAGGCTACGACTAGGCCTG (SEQ ID NO:9)
      (ii) R-Primer (Rho-10) (bold):
         ATGCGCAGGACCCGGTGAGCCA (SEQ ID NO: 10)
   (B) T7 Top (Rho-2) Ta = 38 ◦C: GCCGATAATACGACTCACTATA (SEQ ID NO:2) (italics)
   (C) Taq Ta = 57° C.: Kappa HiFi Ta = 67° C.
      (i) FP w/ T7 (Rho-11):
         GCCGATAATACGACTCACTATAGGCGATGCTAGGCT ACGACTAGGCCTG(SEQ ID NO:11)
(6) Libraries:
   (A) Rho-12
      (i) N45-OSRB-2: (outside) ATGCGCAGGACCCGGTGAGCCANNNNNNNNNNNN NNNNNNNNNNNCTGAGGCGGTTAACCTTGCGCCTC TCCATCANCGCCGAAGCGNGNNNNNNNNNNNNNNN NNNNNNNNCAGGCCTAGTCGTAGCCTAGCATCGCC TATAGTGAGTCGTATTATCGGC (SEQ ID NO:12)
   (B) Rho-13
      (i) N45-ISRB-2: (inside) ATGCGCAGGACCCGGTGAGCCAGGAACCTGNNNN NNNNNNNNNNNNNNNNNNNNNNNNNNNNNNNNNN NNNNNNNTCCATCANCGCCGAAGCGNGGTTCCCAG GCCTAGTCGTAGCCTAGCATCGCCTATAGTGAGTC GTATTATCGGC (SEQ ID NO:13)

### Example 5: Sixty Nucleotide Randomized Region on SRB-2 Aptamer Backbones (Prophetic)

This example describes the SRB-2 aptamer backbone with a randomized 60 nucleotide (N=60) region for binding to larger targets, such as proteins (e.g., spike proteins expressed by a virus). Primers of Example 5 have higher melting peak temperatures compared to the Rho primers of Example 3. Aptamers having randomized regions will be tested with SELEX and may be useful as a switch for THC with the reporter SR-DN. As shown in FIG. 2, the random region will replace or be inserted on the outside stem (circle near bottom) or the inside stem (circle near SR-DN).

The oligonucleotides that will be generated for Example 5 are as follows:
(1) RNA Components:
   (A) Randomized region (60 nts) NNNNNNNNNNNNNNNNNNNNNNNNNNNNNNNNNNNNNNN NNNNNNNNNNNNNNNNNNNNN (SEQ ID NO:165);
   (B) Five Prime Handle (Fh) (bold italics) GGCGATGCTAGGCTACGACTAGGCCTG (SEQ ID NO:9);
   (C) Three Prime Handle (Th) (bold) ATGCGCAGGA CCCGGTGAGCCA (SEQ ID NO: 10);
   (D) T7 Promoter Sequence GCCGATAATACGACTCACTATA (SEQ ID NO:2) (italics);
   (E) SRB2 (aptamer) sequence from Example 1
(2) RNA Domain Substitutions, no T7, no handles:
   (A) N60-OSRB-2 -UA: (outside) NNNNNNNNNNNNNNNNNNNNNNNNNNNNNNCUCGCUUC GGCGAUGAUGGAGAGGCGCAAGGUUAACCGCCUCAGNN NNNNNNNNNNNNNNNNNNNNNNNNNNNN (SEQ ID NO:134)
   (B) N60-OSRB-2-CG: (outside) NNNNNNNNNNNNNNNNNNNNNNNNNNNNNNCCCGCUUC GGCGGUGAUGGAGAGGCGCAAGGUUAACCGCCUCAGNN NNNNNNNNNNNNNNNNNNNNNNNNNNNN (SEQ ID NO:135)
   (C) N60-ISRB-2 -UA: (inside) GGAACCUCGCUUCGGCGAUGAUGGAGNNNNNNNNNNNN NNNNNNNNNNNNNNNNNNNNNNNNNNNNNNNNNNNNNNN NNNNNNNNNCAGGUUCC (SEQ ID NO:137)
   (D) N60-ISRB-2-CG: (inside) GGAACCCCGCUUCGGCGGUGAUGGAGNNNNNNNNNNNN NNNNNNNNNNNNNNNNNNNNNNNNNNNNNNNNNNNNNNN NNNNNNNNNCAGGUUCC (SEQ ID NO:138)
(3) RNA Domain Substitutions with T7 and Handle Additions:
   (A) N60-OSRB-2-NN-T7FR-RNA (outside):
      GCCGAUAAUACGACUCACUAUAGGCGAUGCUAGGCUAC GACUAGGCCUGNNNNNNNNNNNNNNNNNNNNNNNNNNN NNNCNCGCUUCGGCGNUGAUGGAGAGGCGCAAGGUUAA CCGCCUCAGNNNNNNNNNNNNNNNNNNNNNNNNNNNNN NUGGCUCACCGGGUCCUGCGCAU (SEQ ID NO: 157)
   (B) N60-ISRB-2-NN-T7FR-RNA (inside):
      GCCGAUAAUACGACUCACUAUAGGCGAUGCUAGGCUAC GACUAGGCCUGGGAACCNCGCUUCGGCGNUGAUGGANN NNNNNNNNNNNNNNNNNNNNNNNNNNNNNNNNNNNNNNN NNNNNNNNNNNNNNNNNNNCAGGUUCCUGGCUCACCGG GUCCUGCGCAU (SEQ ID NO: 158)
(4) ssDNA (RC RNA) with T7 Promoter and handles:
   (A) N60-OSRB-2-NN-T7FR-DNA (outside):
      ATGCGCAGGACCCGGTGAGCCANNNNNNNNNNNNNNNN NNNNNNNNNNNNNNCTGAGGCGGTTAACCTTGCGCCTCT CCATCANCGCCGAAGCGNGNNNNNNNNNNNNNNNNNNNN NNNNNNNNNNCAGGCCTAGTCGTAGCCTAGCATCGCCTA TAGTGAGTCGTATTATCGGC (SEQ ID NO:14)
   (B) N60-ISRB-2-NN-T7FR-DNA (inside):
      ATGCGCAGGACCCGGTGAGCCAGGAACCTGNNNNNNNN NNNNNNNNNNNNNNNNNNNNNNNNNNNNNNNNNNNNNNN NNNNNNNNNNNNNTCCATCANCGCCGAAGCGNGGTTCCC AGGCCTAGTCGTAGCCTAGCATCGCCTATAGTGAGTCGTA TTATCGGC (SEQ ID NO: 15)
(5) Primers: see Example 4
(6) Libraries:
   (A) Rho-14
      (i) N60-OSRB-2-NN-T7FR-DNA (outside):
         ATGCGCAGGACCCGGTGAGCCANNNNNNNNNNNN NNNNNNNNNNNNNNNNNNCTGAGGCGGTTAACCTT GCGCCTCTCCATCANCGCCGAAGCGNGNNNNNNNN NNNNNNNNNNNNNNNNNNNNNNCAGGCCTAGTCGT AGCCTAGCATCGCCTATAGTGAGTCGTATTATCGGC (SEQ ID NO:14)
   (B) Rho-15
      (i) N60-ISRB-2-NN-T7FR-DNA (inside):
         ATGCGCAGGACCCGGTGAGCCAGGAACCTGNNNN NNNNNNNNNNNNNNNNNNNNNNNNNNNNNNNNNN NNNNNNNNNNNNNNNNNNNNNNTCCATCANCGCCG AAGCGNGGTTCCCAGGCCTAGTCGTAGCCTAGCAT CGCCTATAGTGAGTCGTATTATCGGC (SEQ ID NO:15)

### Example 6: RNA Aptamers Detect Presence of Spike Proteins

This example describes detection of SARS-CoV2 spike proteins by RNA aptamers.

Methods. Reagents were prepared to working concentrations. The solutions were combined in a well plate and exposed to fluorescent light at a wavelength of 575 nm using a plate reader. The fluorophore in the dye, SR-DN, emitted spectra at 602 nm and was used to determine the relative fluorescence between each well. The RNA aptamer was selected for Spike 1 (“S1”) and spike 2 (“S2”) and the false positive control contained S2. Negative control contained RNA aptamer, dye, and no analyte.

At room temperature in ambient light, the following solutions were prepared and combined into various wells of a 96-well white plate, each well at total a volume of 100 µL:
RNA stock: 10 µM in 1 M Tris buffer.
Dye stock DMSO: 2 µM of SR-DN in dimethylsulfoxide (DMSO) diluted to 500 nM in 1 M Tris buffer.
Spike protein stock solution for S1: 390 ng/µL SARS-CoV2 S1 spike protein in 1 M Tris buffer to 100 ng/µL.
Spike protein stock solution for S2: 730 ng/µL SARS-CoV2 S2 spike protein in 1 M Tris buffer to 100 ng/µL.
Log dilutions of S1 and S2 from 10 ng/µL - 10 fg/µL.
Master Mix (“MM”) for each analyte: 1 M Tris buffer, 10 µM RNA in 1 M Tris buffer, 500 nM dye in 1 M Tris buffer, and S1 or S2 analyte in 1 M Tris buffer and bring to 100 µL. see Table 2.

TABLE 2

Dilutions

| MM | 1X (uL) | 23X |
|---|---|---|
| 1X Tris buffer | 35 | 805 |
| RNA [10uM] in Tris | 5 | 115 |
| Dye [500nM] in Tris | 50 | 1,150 |
| Analyte (S1 or S2) [V] in Tris | 10 | --- |
| Total | 100uL | 2070/23 = 90 + 10 uL protein dilutions |

The MM was split into 22 wells following the order in Table 3 and add 10 µL of each of the protein dilutions (e.g., S1, S2 and PBS for no protein). Once solutions were combined in each well, the plate was exposed to fluorescent light at a wavelength of 575 nm. The fluorophore in the dye, SR-DN, emitted spectra at 602 nm.

TABLE 3

Plate Map

| | | | | | | | | | | | |
|---|---|---|---|---|---|---|---|---|---|---|---|
| | 100 fg/uL | | 1 pg/uL | | 10 pg/uL | | 100 pg/uL | | 1 ng/µL | | |
| 1 | 2 | 3 | 4 | 5 | 6 | 7 | 8 | 9 | 10 | 11 | 12 |
| H20 | RNA-S1 | RNA-S1 | RNA-S1 | H20 | RNA-S1 | RNA-S1 | RNA-S1 | RNA-S1 | RNA-S1 | RNA-S1 | H20 |
| H20 | RNA-S2 | RNA-S2 | RNA-S2 | RNA-S2 | RNA-S2 | RNA-S2 | RNA-S2 | RNA-S2 | RNA-S2 | RNA-S2 | H20 |
| H20 | RNA-PBS | RNA-PBS | NRNA-S1 | NRNA-S2 | H20 | H20 | H20 | H20 | H20 | H20 | H20 |
| H20 | H20 | H20 | H20 | H20 | | | | | | | |

TABLE 4

Reaction Layout

| | | | | | | | | | |
|---|---|---|---|---|---|---|---|---|---|
| Well | Reaction Type | Well | Reaction Type | RNA final conc | RNA ID | Dye Final conc | Analyte final conc | Analyte ID | Buffer final conc (I.e, 1x, 0.5x) |
| B2 | Test 1.1 | C2 | Test 6.1 | 500 nM | Rho 91 | 250 nM | 100 fg/uL | Row B - S1 protein | 1X Tris |
| B3 | Test 1.2 | C3 | Test 6.2 | | | | 100 fg/uL | Row C - S2 protein | |
| B4 | Test 2.1 | C4 | Test 7.1 | | | | 1 pg/uL | | |
| B5 | Test 2.2 | C5 | Test 7.2 | | | | 1 pg/uL | | |
| B6 | Test 3.1 | C6 | Test 8.1 | | | | 10 pg/uL | | |
| B7 | Test 3.2 | C7 | Test 8.2 | | | | 10 pg/uL | | |
| B8 | Test 4.1 | C8 | Test 9.1 | | | | 100 pg/uL | | |
| B9 | Test 4.2 | C9 | Test 9.2 | | | | 100 pg/uL | | |
| B10 | Test 5.1 | C10 | Test 10.1 | | | | 1 ng/uL | | |
| B11 | Test 5.2 | C11 | Test 10.2 | | | | 1 ng/uL | | |
| B12 | Water | C12 | Water | | | | | | |
| D2 | Negative Control 1.1 | D3 | Negative Control 1.2 | | | | 0 ng/uL | | |

Conditions tested in each well include (1) water, (2) RNA aptamer with PBS, (3) each of S1 or S2 alone, or (4) dye with each of the two SARS-CoV2 spike proteins S1 or S2, and RNA aptamer.

Results. Compared to control samples, emitted fluorescence decreased for SARS-CoV2 RNA aptamers compared to control upon detection of the corresponding spike proteins. RNA aptamer Rho91 binds SR-DN and fluorescence decreased with increasing concentrations of S2 whereas S1 did not significantly affect fluorophore binding activity as indicated by no change in fluorescence. These data are reflected in Tables 5 to 7. Without intending to be bound by any particular theory, the data suggested competitive binding between the aptamer binding regions on Rho91. The SR-DN binding region interacts with SR-DN with sufficient affinity to increase fluorophore brightness. The analyte binding region of the aptamer recognizes and binds S2 resulting in a proposed conformational change in the SR-DN binding region. The conformational change may have led to dissociation of the aptamer binding region 1 and the SR-DN, therefore decreases brightness. The decrease is fluorescence is proportional to the concentration of S2.

TABLE 5

Results with Rho91 Aptamer and S1

| Rho91 | | |
|---|---|---|
| spike1 [ng/ul] | Average F.U | change |
| 0.00 | 8236.50 | 0.00 |
| 0.01 | 11424.50 | 3188.00 |
| 0.10 | 11940.50 | 3704.00 |
| 1.00 | 11357.00 | 3120.50 |
| 10.00 | 12043.00 | 3806.50 |
| 100.00 | 11630.50 | 3394.00 |
| average | 11105.33 | 3442.60 |
| Cv | 2.61 | 8.85 |

TABLE 6

Results with Rho91 Aptamer and S2

| Rho91 | | |
|---|---|---|
| spike 2 ng/ul | average | change |
| 0.00 | 8236.50 | 0.00 |
| 0.01 | 11467.50 | 3231.00 |
| 0.10 | 11466.50 | 3230.00 |
| 1.00 | 10905.00 | 2668.50 |
| 10.00 | 10284.00 | 2047.50 |
| 100.00 | 9200.00 | 963.50 |
| average | 10259.92 | 2428.10 |
| Cv | 8.94 | 39.26 |

TABLE 7

Differences between S1 and S2 with Rho91 Aptamer

| | Rho91 | | |
|---|---|---|---|
| Target Conc | s1 | s2 | change |
| 0.01 | 3188 | 3231 | -43 |
| 0.10 | 3704 | 3230 | 474 |
| 1.00 | 3120.5 | 2668.5 | 452 |
| 10.00 | 3806.5 | 2047.5 | 1759 |
| 100.00 | 3394 | 963.5 | 2430.5 |

Results. Compared to control samples, emitted fluorescence decreased for SARS-CoV2 RNA aptamers compared to control upon detection of the corresponding spike proteins. RNA aptamer Rho94 (which is encoded by the corresponding Rho-94 DNA molecule shown in Table 1 above) binds SR-DN and fluorescence decreased with increasing concentrations of S2 whereas S1 did not significantly affect fluorophore binding activity as indicated by no change in fluorescence. These data are shown in Table 8 below and methods generally similar to those used above for the Rho91 aptamer were used with the Rho94 aptamer.

TABLE 8

Results with Rho94 Aptamer and S1, and Rho94 Aptamer and S2

| Experiment ID | RNA ID | RNA uM | SR-DN uM France | Spike 1 NA pg/uL | S2 NA REC1807-100 (pg/uL) | Fluorescence Units | Difference | Relative | Percent Change | Average |
|---|---|---|---|---|---|---|---|---|---|---|
| HS4 | Rho94 | 0.5 | 0.25 | 0.1 | 0 | 11384 | 3148 | 1.38 | 38% | 39% |
| HS4 | Rho94 | 0.5 | 0.25 | 0.1 | 0 | 11465 | 3229 | 1.39 | 39% | |
| HS4 | Rho94 | 0.5 | 0.25 | 1 | 0 | 12388 | 4152 | 1.50 | 50% | 45% |
| HS4 | Rho94 | 0.5 | 0.25 | 1 | 0 | 11493 | 3257 | 1.40 | 40% | |
| HS4 | Rho94 | 0.5 | 0.25 | 10 | 0 | 11383 | 3147 | 1.38 | 38% | 38% |
| HS4 | Rho94 | 0.5 | 0.25 | 10 | 0 | 11331 | 3095 | 1.38 | 38% | |
| HS4 | Rho94 | 0.5 | 0.25 | 100 | 0 | 12186 | 3950 | 1.48 | 48% | 46% |
| HS4 | Rho94 | 0.5 | 0.25 | 100 | 0 | 11900 | 3664 | 1.44 | 44% | |
| HS4 | Rho94 | 0.5 | 0.25 | 1000 | 0 | 11561 | 3325 | 1.40 | 40% | 41% |
| HS4 | Rho94 | 0.5 | 0.25 | 1000 | 0 | 11700 | 3464 | 1.42 | 42% | |
| HS4 | Rho94 | 0.5 | 0.25 | 0 | 0.1 | 11222 | 2986 | 1.36 | 36% | 39% |
| HS4 | Rho94 | 0.5 | 0.25 | 0 | 0.1 | 11713 | 3477 | 1.42 | 42% | |
| HS4 | Rho94 | 0.5 | 0.25 | 0 | 1 | 11675 | 3439 | 1.42 | 42% | 39% |
| HS4 | Rho94 | 0.5 | 0.25 | 0 | 1 | 11258 | 3022 | 1.37 | 37% | |
| HS4 | Rho94 | 0.5 | 0.25 | 0 | 10 | 10872 | 2636 | 1.32 | 32% | 32% |
| HS4 | Rho94 | 0.5 | 0.25 | 0 | 10 | 10938 | 2702 | 1.33 | 33% | |
| HS4 | Rho94 | 0.5 | 0.25 | 0 | 100 | 10821 | 2585 | 1.31 | 31% | 25% |
| HS4 | Rho94 | 0.5 | 0.25 | 0 | 100 | 9747 | 1511 | 1.18 | 18% | |
| HS4 | Rho94 | 0.5 | 0.25 | 0 | 1000 | 9540 | 1304 | 1.16 | 16% | 12% |
| HS4 | Rho94 | 0.5 | 0.25 | 0 | 1000 | 8860 | 624 | 1.08 | 8% | |
| HS4 | Rho94 | 0.5 | 0.25 | 0 | 0 | 8791 | 8236.5 | 1.07 | 7% | 0% |
| HS4 | Rho94 | 0.5 | 0.25 | 0 | 0 | 7682 | | 0.93 | -7% | |

### Example 7: COVID-19/SARS-CoV-2 Testing Kit

This example describes detection of SARS-CoV2 spike proteins by RNA aptamers using a testing kit. The assay of the testing kit is a fluorescence-based antigen test designed for use with fluorescence microplate readers capable of fluorescence measurements. The testing kit and assay are intended for detection of SARS-CoV-2 spike protein antigen in anterior nares specimens stored in saline solution from individuals who are suspected of COVID-19. The SARS-CoV-2 assay technology is based on affinity binding of the target, e.g., the SARS-CoV-2 spike protein measurand by an RNA aptamer probe. Probes that can be used in the testing kit include any RNA aptamer described herein.

The SARS-CoV-2 spike antigen is detectable in respiratory specimens during the acute phase of infection. Positive results show the presence of viral antigens, but clinical correlation with patient history and other diagnostic information is necessary to decide infection status. Positive results do not rule out bacterial infection or co-infection with other viruses. Negative results do not rule out SARS-CoV-2 infection and should not be used as the sole basis for treatment or patient management decisions, including infection control decisions. Negative results should be considered in the context of a patient’s recent exposures, history, and the presence of clinical signs and symptoms consistent with COVID-19 and confirmed with a molecular assay, if necessary, for patient management.

Results obtained from the testing kit and assay are to identify SARS-CoV-2 spike antigen in the anterior nares specimen. The anterior nares specimen in saline solution is mixed with sample buffer in wells of the microplate. The microplate has a probe pre-functionalized to the well. Methods of activating microplates and immobilizing the RNA aptamer probe are discussed above in the detailed description. Within the microplate well, the RNA aptamer probe detects and captures the target SARS-CoV-2 antigen if present in the sample. Binding of the antigen by the RNA aptamer promotes access to a second binding domain for a reporter molecule, such as SR-DN. The reporter molecule is composed of a fluorophore attached to a quencher, which is activated upon binding to the probe. The solution is removed, disposed of, and replaced with detection reagent containing the reporter molecule, e.g., SR-DN that is only activated by a RNA aptamer probe bound to the SARS-CoV-2 antigen.

The activation and binding event are stable and determined using a 96-well microplate reader with fluorescence capabilities. The positive determination is made by analyzing the change in fluorescence when a probe and fluorophore are added to the sample. To determine that a significant change has occurred, a baseline fluorescence is generated using a negative control sample. The negative control is an antigen that the probe has been tested against and it has consistently been proven not to interact with. The fluorescence from this sample is shown as a negative result. The positive control is a recombinant version of the protein that consistently generates an increase in fluorescence. This control confirms that the test reagents are functional and capable of generating a positive result.

The SARS-CoV-2 assay is intended for use by trained clinical laboratory personnel specifically instructed and trained in the use of 96-well microplate readers for in vitro diagnostic procedures. Internally validated commercial microplate readers such as the Synergy H1, Hybrid Multi-Mode Reader and the Infinite M1000 Multimode reader. The SARs-CoV-2 assay is currently only intended for use under the Food and Drug Administration’s Emergency Use Authorization. The SARs-CoV-2 test kit is to be used with microplate readers capable of fluorescence, endpoint measurement that can export data to Microsoft Excel for further interpretation and analysis.

Testing Kit Included Materials
Table 2 -Material Components and Specifications

| | |
|---|---|
| Component Name | Component Description |
| Q-LAAD SARS CoV-2 probe plate | Ready-to-use 96-well microplate containing covalently attached probe for the detection of SARS-CoV-2 spike-1 antigen |
| Q-LAAD SARS-CoV-2 Test Buffer | 250 mL, 1x concentrated salt-based buffer for testing |
| Q-LAAD SARS-CoV-2 Assay Detection Reagent | 500 µL, concentrated solution containing reporter molecule in 50% ethanol solvent |
| Clinical Microplate Seals | Transparent sheet coated in acrylate adhesive to minimize any possible contamination during the analyzing of the microplate after completion of the assay |
| Positive Control | Solution of recombinant spike-1 protein in Staphylococcus epidermidis |

Testing Kit. Materials Required but Not Included:
Precision Multichannel pipettes for volumes 5, 10, and 200 microliter volumes.
Disposable pipette tips suitable for the above volumes.
Deionized or distilled water (Molecular grade, RNase-free).
Microplate reader capable of fluorescence measurements (e.g., excitation 575 nm ± 10 nm, emission 610 nm ± 10 nm for optimal spectra with SRB-2 aptamer backbone, other wavelengths may be used with variable results).
Positive control Recombinant protein.
Negative control.

Testing Kit. Recommendations:
Use of this kit should be carried out in a sterile and clean environment.
PPE such as gloves, masks, and goggles should be used to prevent contamination.
Materials found in this kit should be treated as if they were infectious or harmful chemicals.
Use Good Laboratory Practices while carrying out this procedure.

Testing Kit. Instructions. Specimen Collection:
Anterior nares swabs must be collected by a trained and qualified professional.
Sample swabs must be stored in Saline solution. (0.9% NaCI + antibiotics).

From the foregoing, it will be appreciated that specific embodiments of the technology have been described herein for purposes of illustration, but that various modifications may be made without deviating from the scope of the technology. Accordingly, the technology is not limited except as by the appended claims.

### REFERENCES

1. Bouhedda F., Fam K. T., Collot T., AutourA., Marzi S., Klymchenko A., Ryckelynck M. A dimerization-based fluorogenic dye-aptamer module for RNA imaging in live cells. Nat Chem Biol. 2020 Jan; 16(1): 69-76.

2. Holeman LA., Robinson SL., Szostak JW., Wilson C. Isolation and characterization of fluorophore-binding RNA Aptamers. Fold Des. 1998;3(6):423-31.

3. Sunbul M., and Jaschke A. SRB-2: a promiscuous rainbow aptamer for live-cell RNA imaging. Nucleic Acids Res. 2018 Oct 12; 46(18): e110.

4. Arora A., Sunbul M., and Jaschke A. Dual-colour imaging of RNAs using quencher- and fluorophore-binding aptamers. Nucleic Acids Res. 2015 Dec 2; 43(21): e144.

5. Legiewicz M., Lozupone C., Knight R., and Yarus M. Size, constant sequences, and optimal selection. RNA. 2005 Nov; 11(11): 1701-1709.

What is claimed is:

1. A biosensor, comprising:
a reporter; and
an aptamer with at least one stem, the aptamer having -
a target domain comprising a randomized region of at least 30 nucleotides that replace or are disposed within the at least one stem,
a reporter domain configured to bind to the reporter, and
a linker domain between the target domain and the reporter domain.

2. The biosensor of claim 1, wherein the aptamer is an RNA aptamer.

3. The biosensor of claim 1 or 2, wherein the randomized region replaces a first stem of the aptamer.

4. The biosensor of claim 3, wherein the RNA aptamer comprises a nucleotide sequence of GGAACCUN_{Y}AUGAUGGAGAGGCGCAAGGUUAACCGCCUCAGGUUCC (SEQ ID NO:104), GGAACCCN_{Y}GUGAUGGAGAGGCGCAAGGUUAACCGCCUCAGGUUCC (SEQ ID NO:105), or GGAACCNN_{Y}NUGAUGGAGAGGCGCAAGGUUAACCGCCUCAGGUUCC (SEQ ID NO:106).

5. The biosensor of claim 4, wherein the randomized region is represented by N_{Y} in the nucleotide sequence.

6. The biosensor of claim 4 or 5, wherein N_{Y} is NNNNNNNNNNNNNNNNNNNNNNNNNNNNNN (N30) (SEQ ID NO:162), NNNNNNNNNNNNNNNNNNNNNNNNNNNNNNNNNNNNNNNNNNNNN (N45) (SEQ ID NO:163), or NNNNNNNNNNNNNNNNNNNNNNNNNNNNNNNNNNNNNNNNNNNNNNNNNNNN NNNNNNNN (N60) (SEQ ID NO:165).

7. The biosensor of claim 4 or 5, wherein the RNA aptamer has a nucleotide sequence comprising SEQ ID NO:107, SEQ ID NO:108, SEQ ID NO:109, SEQ ID NO:110, SEQ ID NO:111, SEQ ID NO:112, SEQ ID NO:113, SEQ ID NO:114, or SEQ ID NO:115.

8. The biosensor of any one of claims 1 to 7, wherein the randomized region replaces an outside stem of the aptamer.

9. The biosensor of claim 8, wherein the RNA aptamer comprises a nucleotide sequence of N_{Y}CUCGCUUCGGCGAUGAUGGAGAGGCGCAAGGUUAACCGCCUCAGN_{Y}(SEQ ID NO:116), N_{Y}CCCGCUUCGGCGGUGAUGGAGAGGCGCAAGGUUAACCGCCUCAGN_{Y}(SEQ ID NO:117), or N_{Y}CNCGCUUCGGCGNUGAUGGAGAGGCGCAAGGUUAACCGCCUCAGN_{Y}(SEQ ID NO:118).

10. The biosensor of any of claim 8 or 9, wherein the randomized region is 30 nucleotides and N_{Y} is N15.

11. The biosensor of claim 10, wherein the RNA aptamer comprises a nucleotide sequence of NNNNNNNNNNNNNNNCUCGCUUCGGCGAUGAUGGAGAGGCGCAAGGUUAAC CGCCUCAGNNNNNNNNNNNNNNN (SEQ ID NO:122), NNNNNNNNNNNNNNNCCCGCUUCGGCGGUGAUGGAGAGGCGCAAGGUUAAC CGCCUCAGNNNNNNNNNNNNNNN (SEQ ID NO:123), or NNNNNNNNNNNNNNNCNCGCUUCGGCGNUGAUGGAGAGGCGCAAGGUUAAC CGCCUCAGNNNNNNNNNNNNNNN (SEQ ID NO:124).

12. The biosensor of any of claim 8 or 9, wherein the randomized region is 46 nucleotides and N_{Y} is N23.

13. The biosensor of claim 12, wherein the RNA aptamer comprises a nucleotide sequence of NNNNNNNNNNNNNNNNNNNNNNNCUCGCUUCGGCGAUGAUGGAGAGGCGCA AGGUUAACCGCCUCAGNNNNNNNNNNNNNNNNNNNNNNN (SEQ ID NO:128), NNNNNNNNNNNNNNNNNNNNNNNCCCGCUUCGGCGGUGAUGGAGAGGCGCA AGGUUAACCGCCUCAGNNNNNNNNNNNNNNNNNNNNNNN (SEQ ID NO:129), or NNNNNNNNNNNNNNNNNNNNNNNCNCGCUUCGGCGNUGAUGGAGAGGCGCA AGGUUAACCGCCUCAGNNNNNNNNNNNNNNNNNNNNNNN (SEQ ID NO:130).

14. The biosensor of any of claim 8 or 9, wherein the randomized region is 60 nucleotides and N_{Y} is N30.

15. The biosensor of claim 14, wherein the RNA aptamer comprises a nucleotide sequence of NNNNNNNNNNNNNNNNNNNNNNNNNNNNNNCUCGCUUCGGCGAUGAUGGAG AGGCGCAAGGUUAACCGCCUCAGNNNNNNNNNNNNNNNNNNNNNNNNNNNNN N (SEQ ID NO:134), NNNNNNNNNNNNNNNNNNNNNNNNNNNNNNCCCGCUUCGGCGGUGAUGGAG AGGCGCAAGGUUAACCGCCUCAGNNNNNNNNNNNNNNNNNNNNNNNNNNNNN N (SEQ ID NO:135), or NNNNNNNNNNNNNNNNNNNNNNNNNNNNNNCNCGCUUCGGCGNUGAUGGAG AGGCGCAAGGUUAACCGCCUCAGNNNNNNNNNNNNNNNNNNNNNNNNNNNNN N (SEQ ID NO:136).

16. The biosensor of any one of claims 1 to 15, wherein the randomized region replaces an inside stem of the aptamer.

17. The biosensor of claim 16, wherein the RNA aptamer comprises a nucleotide sequence of GGAACCUCGCUUCGGCGAUGAUGGAGN_{Y}CAGGUUCC (SEQ ID NO:119), GGAACCCCGCUUCGGCGGUGAUGGAGN_{Y}CAGGUUCC (SEQ ID NO:120), or GGAACCNCGCUUCGGCGNUGAUGGAGN_{Y}CAGGUUCC (SEQ ID NO:121).

18. The biosensor of claim 16 or 17, wherein the randomized region is 30 nucleotides and N_{Y} is N30.

19. The biosensor of claim 18, wherein the RNA aptamer comprises a nucleotide sequence of GGAACCUCGCUUCGGCGAUGAUGGAGNNNNNNNNNNNNNNNNNNNNNNNNN NNNNNCAGGUUCC (SEQ ID NO:125), GGAACCCCGCUUCGGCGGUGAUGGAGNNNNNNNNNNNNNNNNNNNNNNNNN NNNNNCAGGUUCC (SEQ ID NO:126), or GGAACCNCGCUUCGGCGNUGAUGGAGNNNNNNNNNNNNNNNNNNNNNNNNN NNNNNCAGGUUCC (SEQ ID NO:127).

20. The biosensor of any one of claims 16 or 17, wherein the randomized region is 45 nucleotides and N_{Y} is N45.

21. The biosensor of claim 20, wherein the RNA aptamer comprises a nucleotide sequence of GGAACCUCGCUUCGGCGAUGAUGGAGNNNNNNNNNNNNNNNNNNNNNNNNN NNNNNNNNNNNNNNNNNNNNCAGGUUCC (SEQ ID NO:131), GGAACCCCGCUUCGGCGGUGAUGGAGNNNNNNNNNNNNNNNNNNNNNNNNN NNNNNNNNNNNNNNNNNNNNCAGGUUCC (SEQ ID NO:132), or GGAACCNCGCUUCGGCGNUGAUGGAGNNNNNNNNNNNNNNNNNNNNNNNNN NNNNNNNNNNNNNNNNNNNNCAGGUUCC (SEQ ID NO:133).

22. The biosensor of any of claims 16 or 17, wherein the randomized region is 60 nucleotides and N_{Y} is N60.

23. The biosensor of claim 20, wherein the RNA aptamer comprises a nucleotide sequence of GGAACCUCGCUUCGGCGAUGAUGGAGNNNNNNNNNNNNNNNNNNNNNNNNN NNNNNNNNNNNNNNNNNNNNNNNNNNNNNNNNNNNCAGGUUCC(SEQ ID NO:137), GGAACCCCGCUUCGGCGGUGAUGGAGNNNNNNNNNNNNNNNNNNNNNNNNN NNNNNNNNNNNNNNNNNNNNNNNNNNNNNNNNNNNCAGGUUCCGGAACCU (SEQ ID NO:138), or GGAACCNCGCUUCGGCGNUGAUGGAGNNNNNNNNNNNNNNNNNNNNNNNNN NNNNNNNNNNNNNNNNNNNNNNNNNNNNNNNNNNNCAGGUUCC(SEQ ID NO:139).

24. The biosensor of any one of the preceding claims, wherein the nucleotide sequence of the randomized region in the target domain that binds the target is identified by SELEX.

25. The biosensor of any one of the preceding claims, wherein the reporter is a fluorescent molecule.

26. The biosensor of claim 25, wherein the fluorescent molecule is sulforhodamine-dinitroaniline.

27. The biosensor of any one of the preceding claims, wherein the linker is operably connected between the target domain and the reporter domain such that a conformational change occurs in the RNA aptamer in response to the target domain binding to a target, the reporter binding to the reporter domain, or a combination thereof.

28. A method of detecting a target in sample comprising contacting the sample with the biosensor of any one of the preceding claims.

29. The method of claim 28, wherein the target is a pathogen, a small molecule, a solvent, or an ion.

30. The method of claim 29, wherein the pathogen is a bacterial pathogen, a viral pathogen, a prokaryotic pathogen, a fungal pathogen, or a combination thereof.

31. The method of claim 29, wherein the pathogen is adenovirus, coronavirus, human metapneumovirus, human rhinovirus/enterovirus, influenza, parainfluenza, respiratory syncytial virus, bordatella pertussis, chlamydophia penumoniae, SARS-CoV, SARS-CoV2, MERS-CoV, UPEC, E. coli, klebsiella pneumoniae, proteus mirabilis, pseudomonas aeruginosa, staphylococcus saprophyticus, enterococcus faecalis, enterococcus faecim, clostridioides difficile, methicillin-resistant staphylococcus aureus, proteins synthesized by antibiotic resistant bacteria, West Nile virus, Zika virus, Ebola virus, salmonella, equine herpesvirus type I) and type IV, human immunodeficiency virus (HIV), hepatitis A, hepatitis B, hepatitis C, malaria, Dengue virus, norovirus, rotavirus, astrovirus, Marburg virus, rabies, small pox, measles, or hantavirus.

32. The method of claim 31, wherein the target is a protein encoded by the SARS-CoV2 pathogen.

33. The method of claim 32, wherein the protein is a spike protein.

34. The method of claim 29, wherein the small molecule is a toxin or a pharmaceutical agent.

35. The method of claim 34, wherein the small molecule is a cannabinoid, bisphenol A, fluoride, or benzene.

36. The method of claim 35, wherein the cannabinoid is cannabidiol, cannabinol, or tetrahydrocannabinol.

37. The method of claim 29, wherein the solvent is acetone, cyclohexane, acetic acid, ethanol, or benzene.

38. The method of claim 29, wherein the ion is potassium, chloride, sodium, lithium, magnesium, mercury, or lead.


SEQUENCE LISTING (166 sequences)

SEQ ID NO: 1 - DNA, 19 bp, Artificial Sequence
  Synthetic
atgcatgtccggactgcca

SEQ ID NO: 2 - DNA, 22 bp, Artificial Sequence
  Synthetic
gccgataatacgactcactata

SEQ ID NO: 3 - DNA, 45 bp, Artificial Sequence
  Synthetic
gccgataatacgactcactataggtagccctgcccggtgtacctg

SEQ ID NO: 4 - DNA, 138 bp, Artificial Sequence
  Synthetic
  misc_feature (20)..(34): n is a, c, g, or t
  misc_feature (79)..(93): n is a, c, g, or t
atgcatgtccggactgccannnnnnnnnnnnnnnctgaggcggttaaccttgcgcctctccatcatcgccgaagcgagnnnnnnnnnnnnnnncaggtacaccgggcagggctacctatagtgagtcgtattatcggc

SEQ ID NO: 5 - DNA, 138 bp, Artificial Sequence
  Synthetic
  misc_feature (20)..(34): n is a, c, g, or t
  misc_feature (79)..(93): n is a, c, g, or t
atgcatgtccggactgccannnnnnnnnnnnnnnctgaggcggttaaccttgcgcctctccatcaccgccgaagcgggnnnnnnnnnnnnnnncaggtacaccgggcagggctacctatagtgagtcgtattatcggc

SEQ ID NO: 6 - DNA, 128 bp, Artificial Sequence
  Synthetic
  misc_feature (28)..(57): n is a, c, g, or t
atgcatgtccggactgccaggaacctgnnnnnnnnnnnnnnnnnnnnnnnnnnnnnnctccatcatcgccgaagcgaggttcccaggtacaccgggcagggctacctatagtgagtcgtattatcggc

SEQ ID NO: 7 - DNA, 128 bp, Artificial Sequence
  Synthetic
  misc_feature (28)..(57): n is a, c, g, or t
atgcatgtccggactgccaggaacctgnnnnnnnnnnnnnnnnnnnnnnnnnnnnnnctccatcaccgccgaagcggggttcccaggtacaccgggcagggctacctatagtgagtcgtattatcggc

SEQ ID NO: 8 - DNA, 23 bp, Artificial Sequence
  Synthetic
ggtagccctgcccggtgtacctg

SEQ ID NO: 9 - DNA, 27 bp, Artificial Sequence
  Synthetic
ggcgatgctaggctacgactaggcctg

SEQ ID NO: 10 - DNA, 22 bp, Artificial Sequence
  Synthetic
atgcgcaggacccggtgagcca

SEQ ID NO: 11 - DNA, 49 bp, Artificial Sequence
  Synthetic
gccgataatacgactcactataggcgatgctaggctacgactaggcctg

SEQ ID NO: 12 - DNA, 161 bp, Artificial Sequence
  Synthetic
  misc_feature (23)..(45): n is a, c, g, or t
  misc_feature (77)..(77): n is a, c, g, or t
  misc_feature (88)..(88): n is a, c, g, or t
  misc_feature (90)..(112): n is a, c, g, or t
atgcgcaggacccggtgagccannnnnnnnnnnnnnnnnnnnnnnctgaggcggttaaccttgcgcctctccatcancgccgaagcgngnnnnnnnnnnnnnnnnnnnnnnncaggcctagtcgtagcctagcatcgcctatagtgagtcgtattatcggc

SEQ ID NO: 13 - DNA, 149 bp, Artificial Sequence
  Synthetic
  misc_feature (31)..(75): n is a, c, g, or t
  misc_feature (83)..(83): n is a, c, g, or t
  misc_feature (94)..(94): n is a, c, g, or t
atgcgcaggacccggtgagccaggaacctgnnnnnnnnnnnnnnnnnnnnnnnnnnnnnnnnnnnnnnnnnnnnntccatcancgccgaagcgnggttcccaggcctagtcgtagcctagcatcgcctatagtgagtcgtattatcggc

SEQ ID NO: 14 - DNA, 175 bp, Artificial Sequence
  Synthetic
  misc_feature (23)..(52): n is a, c, g, or t
  misc_feature (84)..(84): n is a, c, g, or t
  misc_feature (95)..(95): n is a, c, g, or t
  misc_feature (97)..(126): n is a, c, g, or t
atgcgcaggacccggtgagccannnnnnnnnnnnnnnnnnnnnnnnnnnnnnctgaggcggttaaccttgcgcctctccatcancgccgaagcgngnnnnnnnnnnnnnnnnnnnnnnnnnnnnnncaggcctagtcgtagcctagcatcgcctatagtgagtcgtattatcggc

SEQ ID NO: 15 - DNA, 164 bp, Artificial Sequence
  Synthetic
  misc_feature (31)..(90): n is a, c, g, or t
  misc_feature (98)..(98): n is a, c, g, or t
  misc_feature (109)..(109): n is a, c, g, or t
atgcgcaggacccggtgagccaggaacctgnnnnnnnnnnnnnnnnnnnnnnnnnnnnnnnnnnnnnnnnnnnnnnnnnnnnnnnnnnnntccatcancgccgaagcgnggttcccaggcctagtcgtagcctagcatcgcctatagtgagtcgtattatcggc

SEQ ID NO: 16 - DNA, 74 bp, Artificial Sequence
  Synthetic
tcgtcggcagcgtcagatgtgtataagagacaggcatgcatgcatgcatgcggtagccctgcccggtgtacctg

SEQ ID NO: 17 - DNA, 71 bp, Artificial Sequence
  Synthetic
tcgtcggcagcgtcagatgtgtataagagacagtgcatgcatgcatgcggtagccctgcccggtgtacctg

SEQ ID NO: 18 - DNA, 68 bp, Artificial Sequence
  Synthetic
tcgtcggcagcgtcagatgtgtataagagacagatgcatgcatgcggtagccctgcccggtgtacctg

SEQ ID NO: 19 - DNA, 65 bp, Artificial Sequence
  Synthetic
tcgtcggcagcgtcagatgtgtataagagacagcatgcatgcggtagccctgcccggtgtacctg

SEQ ID NO: 20 - DNA, 53 bp, Artificial Sequence
  Synthetic
gtctcgtgggctcggagatgtgtataagagacagatgcatgtccggactgcca

SEQ ID NO: 21 - DNA, 78 bp, Artificial Sequence
  Synthetic
tcgtcggcagcgtcagatgtgtataagagacaggcatgcatgcatgcatgcggcgatgctaggctacgactaggcctg

SEQ ID NO: 22 - DNA, 75 bp, Artificial Sequence
  Synthetic
tcgtcggcagcgtcagatgtgtataagagacagtgcatgcatgcatgcggcgatgctaggctacgactaggcctg

SEQ ID NO: 23 - DNA, 72 bp, Artificial Sequence
  Synthetic
tcgtcggcagcgtcagatgtgtataagagacagatgcatgcatgcggcgatgctaggctacgactaggcctg

SEQ ID NO: 24 - DNA, 69 bp, Artificial Sequence
  Synthetic
tcgtcggcagcgtcagatgtgtataagagacagcatgcatgcggcgatgctaggctacgactaggcctg

SEQ ID NO: 25 - DNA, 56 bp, Artificial Sequence
  Synthetic
gtctcgtgggctcggagatgtgtataagagacagatgcgcaggacccggtgagcca

SEQ ID NO: 26 - DNA, 109 bp, Artificial Sequence
  Synthetic DNA/RNA
ggaaccucgcuucggcgaugauggagauuuatccagagtgacgcagcatttcatcgggtccaaaaggggctgctcgggattgcggatatggacacgtauuucagguucc

SEQ ID NO: 27 - RNA, 109 nt, Artificial Sequence
  Synthetic
  modified_base (32)..(32): ideoxyU
  modified_base (39)..(39): ideoxyU
  modified_base (49)..(51): ideoxyU
  modified_base (54)..(54): ideoxyU
  modified_base (59)..(59): ideoxyU
  modified_base (71)..(71): ideoxyU
  modified_base (74)..(74): ideoxyU
  modified_base (80)..(81): ideoxyU
  modified_base (87)..(87): ideoxyU
  modified_base (89)..(89): ideoxyU
  modified_base (97)..(97): ideoxyU
ggaaccucgcuucggcgaugauggagauuuauccagagugacgcagcauuucaucggguccaaaaggggcugcucgggauugcggauauggacacguauuucagguucc

SEQ ID NO: 28 - RNA, 109 nt, Artificial Sequence; identical to SEQ ID NO: 27
  Synthetic

SEQ ID NO: 29 - DNA, 93 bp, Artificial Sequence
  Synthetic DNA/RNA
ggaaccccgcuucggcggugauggagauuucagcaccgaccttgtgctttgggagtgctggtccaagggcgttaatggacaauuucagguucc

SEQ ID NO: 30 - RNA, 93 nt, Artificial Sequence
  Synthetic
  modified_base (42)..(43): ideoxyU
  modified_base (45)..(45): ideoxyU
  modified_base (48)..(50): ideoxyU
  modified_base (59)..(59): ideoxyU
  modified_base (62)..(62): ideoxyU
  modified_base (72)..(73): ideoxyU
  modified_base (76)..(76): ideoxyU
ggaaccccgcuucggcggugauggagauuucagcaccgaccuugugcuuugggagugcugguccaagggcguuaauggacaauuucagguucc

SEQ ID NO: 31 - RNA, 93 nt, Artificial Sequence; identical to SEQ ID NO: 30
  Synthetic

SEQ ID NO: 32 - DNA, 51 bp, Artificial Sequence
  Synthetic
  misc_feature (1)..(1): 5FluorT
cagcaccgaccttgtgctttgggagtgctggtccaagggcgttaatggaca

SEQ ID NO: 33 - DNA, 67 bp, Artificial Sequence
  Synthetic
  misc_feature (1)..(1): 5FluorT
atccagagtgacgcagcatttcatcgggtccaaaaggggctgctcgggattgcggatatggacacgt

SEQ ID NO: 34 - DNA, 76 bp, Artificial Sequence
  Synthetic
ggaacctgaggcggttaaccttgcgcctctccatcaccgccgaagcggggttcctatagtgagtcgtattatcggc

SEQ ID NO: 35 - DNA, 138 bp, Artificial Sequence
  Synthetic
atgcatgtccggactgccacctgcaagccacgcgctgaggcggttaaccttgcgcctctccatcatcgccgaagcgagcgcgggcagtagccgcaggtacaccgggcagggctacctatagtgagtcgtattatcggc

SEQ ID NO: 36 - DNA, 96 bp, Artificial Sequence
  Synthetic
cctgcaagccacgcgctgaggcggttaaccttgcgcctctccatcatcgccgaagcgagcgcgggcagtagccgtatagtgagtcgtattatcggc

SEQ ID NO: 37 - DNA, 138 bp, Artificial Sequence
  Synthetic
atgcatgtccggactgccaggtgcaccgattggtctgaggcggttaaccttgcgcctctccatcatcgccgaagcgagaaccaattgcgagagcaggtacaccgggcagggctacctatagtgagtcgtattatcggc

SEQ ID NO: 38 - DNA, 96 bp, Artificial Sequence
  Synthetic
ggtgcaccgattggtctgaggcggttaaccttgcgcctctccatcatcgccgaagcgagaaccaattgcgagagtatagtgagtcgtattatcggc

SEQ ID NO: 39 - DNA, 138 bp, Artificial Sequence
  Synthetic
atgcatgtccggactgccagaacggtagttagtgctgaggcggttaaccttgcgcctctccatcatcgccgaagcgaggcaattaacttggtacaggtacaccgggcagggctacctatagtgagtcgtattatcggc

SEQ ID NO: 40 - DNA, 96 bp, Artificial Sequence
  Synthetic
gaacggtagttagtgctgaggcggttaaccttgcgcctctccatcatcgccgaagcgaggcaattaacttggtatatagtgagtcgtattatcggc

SEQ ID NO: 41 - DNA, 138 bp, Artificial Sequence
  Synthetic
  misc_feature (20)..(34): n is a, c, g, or t
  misc_feature (66)..(66): n is a, c, g, or t
  misc_feature (77)..(77): n is a, c, g, or t
  misc_feature (79)..(93): n is a, c, g, or t
atgcatgtccggactgccannnnnnnnnnnnnnnctgaggcggttaaccttgcgcctctccatcancgccgaagcgngnnnnnnnnnnnnnnncaggtacaccgggcagggctacctatagtgagtcgtattatcggc

SEQ ID NO: 42 - DNA, 128 bp, Artificial Sequence
  Synthetic
  misc_feature (28)..(57): n is a, c, g, or t
  misc_feature (66)..(66): n is a, c, g, or t
  misc_feature (77)..(77): n is a, c, g, or t
atgcatgtccggactgccaggaacctgnnnnnnnnnnnnnnnnnnnnnnnnnnnnnnctccatcancgccgaagcgnggttcccaggtacaccgggcagggctacctatagtgagtcgtattatcggc

SEQ ID NO: 43 - DNA, 138 bp, Artificial Sequence
  Synthetic
atgcatgtccggactgccatccgaaacgggcactctgaggcggttaaccttgcgcctctccatcatcgccgaagcgaggagtgccagctgccccaggtacaccgggcagggctacctatagtgagtcgtattatcggc

SEQ ID NO: 44 - DNA, 96 bp, Artificial Sequence
  Synthetic
tccgaaacgggcactctgaggcggttaaccttgcgcctctccatcatcgccgaagcgaggagtgccagctgccctatagtgagtcgtattatcggc

SEQ ID NO: 45 - DNA, 138 bp, Artificial Sequence
  Synthetic
atgcatgtccggactgccaatccgaacggcggatctgaggcggttaaccttgcgcctctccatcatcgccgaagcgaggatccgcatactgcccaggtacaccgggcagggctacctatagtgagtcgtattatcggc

SEQ ID NO: 46 - DNA, 96 bp, Artificial Sequence
  Synthetic
atccgaacggcggatctgaggcggttaaccttgcgcctctccatcatcgccgaagcgaggatccgcatactgcctatagtgagtcgtattatcggc

SEQ ID NO: 47 - DNA, 138 bp, Artificial Sequence
  Synthetic
atgcatgtccggactgccacgatgacccgcagagctgaggcggttaaccttgcgcctctccatcatcgccgaagcgaggctcaattcaaaacgcaggtacaccgggcagggctacctatagtgagtcgtattatcggc

SEQ ID NO: 48 - DNA, 138 bp, Artificial Sequence
  Synthetic
atgcatgtccggactgccaggtgtacgaatgagcctgaggcggttaaccttgcgcctctccatcatcgccgaagcgagggctcaactgtagaacaggtacaccgggcagggctacctatagtgagtcgtattatcggc

SEQ ID NO: 49 - DNA, 138 bp, Artificial Sequence
  Synthetic
atgcatgtccggactgccaggggtacctgagctcctgaggcggttaaccttgcgcctctccatcatcgccgaagcgaggagcttggtaacgttcaggtacaccgggcagggctacctatagtgagtcgtattatcggc

SEQ ID NO: 50 - DNA, 138 bp, Artificial Sequence
  Synthetic
atgcatgtccggactgccaggtgtgccaacgcggctgaggcggttaaccttgcgcctctccatcatcgccgaagcgagaccgcgtggtaggatcaggtacaccgggcagggctacctatagtgagtcgtattatcggc

SEQ ID NO: 51 - DNA, 138 bp, Artificial Sequence
  Synthetic
atgcatgtccggactgccagggtacccgggacgcctgaggcggttaaccttgcgcctctccatcatcgccgaagcgaggcgtcgatttagtagcaggtacaccgggcagggctacctatagtgagtcgtattatcggc

SEQ ID NO: 52 - DNA, 24 bp, Artificial Sequence
  Synthetic
cctgcaagccacgcgctgaggcgg

SEQ ID NO: 53 - DNA, 42 bp, Artificial Sequence
  Synthetic
gccgataatacgactcactatacggctactgcccgcgctcgc

SEQ ID NO: 54 - DNA, 22 bp, Artificial Sequence
  Synthetic
gggtacccgggacgcctgaggc

SEQ ID NO: 55 - DNA, 48 bp, Artificial Sequence
  Synthetic
gccgataatacgactcactatactactaaatcgacgcctcgcttcggc

SEQ ID NO: 56 - DNA, 109 bp, Artificial Sequence
  Synthetic
ggaacctgaaatacgtgtccatatccgcaatcccgagcagccccttttggacccgatgaaatgctgcgtcactctggataaatctccatcatcgccgaagcgaggttcc

SEQ ID NO: 57 - DNA, 26 bp, Artificial Sequence
  Synthetic
ggaacctgaaatacgtgtccatatcc

SEQ ID NO: 58 - DNA, 39 bp, Artificial Sequence
  Synthetic
gccgataatacgactcactataggaacctcgcttcggcg

SEQ ID NO: 59 - DNA, 93 bp, Artificial Sequence
  Synthetic
ggaacctgaaattgtccattaacgcccttggaccagcactcccaaagcacaaggtcggtgctgaaatctccatcaccgccgaagcggggttcc

SEQ ID NO: 60 - DNA, 26 bp, Artificial Sequence
  Synthetic
ggaacctgaaattgtccattaacgcc

SEQ ID NO: 61 - DNA, 118 bp, Artificial Sequence
  Synthetic
atgcatgtccggactgccaggaacctgaggcggttaaccttgcgcctctccatcatcgccgaagcgaggttcccaggtacaccgggcagggctacctatagtgagtcgtattatcggc

SEQ ID NO: 62 - DNA, 76 bp, Artificial Sequence
  Synthetic
ggaacctgaggcggttaaccttgcgcctctccatcatcgccgaagcgaggttcctatagtgagtcgtattatcggc

SEQ ID NO: 63 - DNA, 175 bp, Artificial Sequence
  Synthetic
atgcgcaggacccggtgagccagccctagggcggggcttccttggttatcgtctgaggcggttaaccttgcgcctctccatcagcgccgaagcgcgacgacaggatgctgagggcggacaggttggcaggcctagtcgtagcctagcatcgcctatagtgagtcgtattatcggc

SEQ ID NO: 64 - DNA, 161 bp, Artificial Sequence
  Synthetic
atgcgcaggacccggtgagccatcttcggtgcagccgcgtagcagctgaggcggttaaccttgcgcctctccatcagcgccgaagcgagagccttgactacgattaggttggcaggcctagtcgtagcctagcatcgcctatagtgagtcgtattatcggc

SEQ ID NO: 65 - DNA, 161 bp, Artificial Sequence
  Synthetic
atgcgcaggacccggtgagccacacggcggtacgggcacggtatcctgaggcggttaaccttgcgcctctccatcagcgccgaagcgcggcataatcgattaggcccgggcgcaggcctagtcgtagcctagcatcgcctatagtgagtcgtattatcggc

SEQ ID NO: 66 - DNA, 161 bp, Artificial Sequence
  Synthetic
atgcgcaggacccggtgagccagcgggcactcaaatcgatcaaatctgaggcggttaaccttgcgcctctccatcagcgccgaagcgcgaaacgatcgtttgcggagctactcaggcctagtcgtagcctagcatcgcctatagtgagtcgtattatcggc

SEQ ID NO: 67 - DNA, 161 bp, Artificial Sequence
  Synthetic
atgcgcaggacccggtgagccaacgggggcaatctgcgtcgcagcctgaggcggttaaccttgcgcctctccatcagcgccgaagcgcggccactgacgacgtaggtcgcaacaggcctagtcgtagcctagcatcgcctatagtgagtcgtattatcggc

SEQ ID NO: 68 - DNA, 161 bp, Artificial Sequence
  Synthetic
atgcgcaggacccggtgagccagacggatcaatggcctgtgtggactgaggcggttaaccttgcgcctctccatcagcgccgaagcgcgtaccaccgtaggtgaggtttactcaggcctagtcgtagcctagcatcgcctatagtgagtcgtattatcggc

SEQ ID NO: 69 - DNA, 175 bp, Artificial Sequence
  Synthetic
atgcgcaggacccggtgagccacgtggcagaggtttaccatcggaccgggagctgaggcggttaaccttgcgcctctccatcagcgccgaagcgcgagctccatgcgaaggctacggacgaggtggcaggcctagtcgtagcctagcatcgcctatagtgagtcgtattatcggc

SEQ ID NO: 70 - DNA, 161 bp, Artificial Sequence
  Synthetic
atgcgcaggacccggtgagccattagcgaatgatcacacgggtgactgaggcggttaaccttgcgcctctccatcagcgccgaagcgaggccagcgaccttggccgtcgacccaggcctagtcgtagcctagcatcgcctatagtgagtcgtattatcggc

SEQ ID NO: 71 - DNA, 138 bp, Artificial Sequence
  Synthetic
atgcatgtccggactgccagatgtaccagcgcaactgaggcggttaaccttgcgcctctccatcatcgccgaagcgaggttgcgcgttggattcaggtacaccgggcagggctacctatagtgagtcgtattatcggc

SEQ ID NO: 72 - DNA, 138 bp, Artificial Sequence
  Synthetic
atgcatgtccggactgccacggtgtacaacagggctgaggcggttaaccttgcgcctctccatcatcgccgaagcgaggctctgcgtctttttcaggtacaccgggcagggctacctatagtgagtcgtattatcggc

SEQ ID NO: 73 - DNA, 138 bp, Artificial Sequence
  Synthetic
atgcatgtccggactgccaggtgtaccatagacgctgaggcggttaaccttgcgcctctccatcatcgccgaagcgaggcgtctacagaaaagcaggtacaccgggcagggctacctatagtgagtcgtattatcggc

SEQ ID NO: 74 - DNA, 138 bp, Artificial Sequence
  Synthetic
atgcatgtccggactgccaggcataactgatgtgctgaggcggttaaccttgcgcctctccatcatcgccgaagcgagcacattggtaacgttcaggtacaccgggcagggctacctatagtgagtcgtattatcggc

SEQ ID NO: 75 - DNA, 143 bp, Artificial Sequence
  Synthetic
atgcatgtccggactgccacgtcctgccacctgtctgaactgaggcggttaaccttgcgcctctccatcatcgccgaagcgagtttcagacatcggggcaggtacaccgggcagggctacctatagtgagtcgtattatcggc

SEQ ID NO: 76 - DNA, 128 bp, Artificial Sequence
  Synthetic
atgcatgtccggactgccaggaacctgttaacctcgtggtagcatcggcgagtggaactccatcatcgccgaagcgaggttcccaggtacaccgggcagggctacctatagtgagtcgtattatcggc

SEQ ID NO: 77 - DNA, 128 bp, Artificial Sequence
  Synthetic
atgcatgtccggactgccaggaacctgggaaaccgcgttacaacgagatggggtaacctccatcatcgccgaagcgaggttcccaggtacaccgggcagggctacctatagtgagtcgtattatcggc

SEQ ID NO: 78 - DNA, 138 bp, Artificial Sequence
  Synthetic
atgcatgtccggactgccaagcgcaccggccttactgaggcggttaaccttgcgcctctccatcatcgccgaagcgagtaagtcgatcgagatcaggtacaccgggcagggctacctatagtgagtcgtattatcggc

SEQ ID NO: 79 - DNA, 138 bp, Artificial Sequence
  Synthetic
atgcatgtccggactgccaagtgtaccgactgtgctgaggcggttaaccttgcgcctctccatcatcgccgaagcgagcacagtgatagctttcaggtacaccgggcagggctacctatagtgagtcgtattatcggc

SEQ ID NO: 80 - DNA, 138 bp, Artificial Sequence
  Synthetic
atgcatgtccggactgccaggtgtacgaatcgcactgaggcggttaaccttgcgcctctccatcatcgccgaagcgagtgcgatcggatagctcaggtacaccgggcagggctacctatagtgagtcgtattatcggc

SEQ ID NO: 81 - DNA, 138 bp, Artificial Sequence
  Synthetic
atgcatgtccggactgccaagtgtacctcttccgctgaggcggttaacattgcgcctctccatcaccgccgaagcgggcggaagccttagccacaggtacaccgggcagggctacctatagtgagtcgtattatcggc

SEQ ID NO: 82 - DNA, 161 bp, Artificial Sequence
  Synthetic
atgcgcaggacccggtgagccagtcgagccctagctcattgcgtactgaggcggttaaccttgcgcctctccatcagcgccgaagcgcgtacgcgtagtgatgagtacgactcaggcctagtcgtagcctagcatcgcctatagtgagtcgtattatcggc

SEQ ID NO: 83 - DNA, 161 bp, Artificial Sequence
  Synthetic
atgcgcaggacccggtgagccaagccgagtgatgccgcatggactctgaggcggttaaccttgcgcctctccatcagcgccgaagcgcgcaagtccatgggatgtaagtacgcaggcctagtcgtagcctagcatcgcctatagtgagtcgtattatcggc

SEQ ID NO: 84 - DNA, 161 bp, Artificial Sequence
  Synthetic
atgcgcaggacccggtgagccagtcgggtcgtggcgtaacacactctgaggcggttaaccttgcgcctctccatcagcgccgaagcgcgaagtggtagtgctagcacttagacaggcctagtcgtagcctagcatcgcctatagtgagtcgtattatcggc

SEQ ID NO: 85 - DNA, 161 bp, Artificial Sequence
  Synthetic
atgcgcaggacccggtgagccagaaagcgaatgggttcacgagtactgaggcggttaaccttgcgcctctccatcagcgccgaagcgaggttgtcctaggtctggtgacgaacaggcctagtcgtagcctagcatcgcctatagtgagtcgtattatcggc

SEQ ID NO: 86 - DNA, 161 bp, Artificial Sequence
  Synthetic
atgcgcaggacccggtgagccagtggcggtaaccgcacccgggttctgaggcggttaaccttgcgcctctccatcagcgccgaagcgaggggacgaccaacaggcttggcttcaggcctagtcgtagcctagcatcgcctatagtgagtcgtattatcggc

SEQ ID NO: 87 - DNA, 161 bp, Artificial Sequence
  Synthetic
atgcgcaggacccggtgagccacacgaatagtgagggtcgaaatcctgaggcggttagccttgcgcctctccatcagcgccgaagcgtgtctacgattagggacctgtggcgcaggcctagtcgtagcctagcatcgcctatagtgagtcgtattatcggc

SEQ ID NO: 88 - DNA, 161 bp, Artificial Sequence
  Synthetic
atgcgcaggacccggtgagccataacgggtccacgattgctaagactgaggcggttaaccttgcgcctctccatcaacgccgaagcgtgcaacttgttttggctgacaagcgcaggcctagtcgtagcctagcatcgcctatagtgagtcgtattatcggc

SEQ ID NO: 89 - DNA, 161 bp, Artificial Sequence
  Synthetic
atgcgcaggacccggtgagccacggcgaaagttatcacgggtaacctgaggcggttaaccttgcgcctctccatcagcgccgaagcgtggcaacgactgagtctgtcggcadcaggcctagtcgtagcctagcatcgcctatagtgagtcgtattatcggc

SEQ ID NO: 90 - DNA, 161 bp, Artificial Sequence
  Synthetic
atgcgcaggacccggtgagccagcggcaggtggctcgccgcaagtctgaggcggttaaccttgcgcctctccatcagcgccgaagcgcgacttgatgctcggttgtatcgaacaggcctagtcgtagcctagcatcgcctatagtgagtcgtattatcggc

SEQ ID NO: 91 - DNA, 161 bp, Artificial Sequence
  Synthetic
atgcgcaggacccggtgagccaaacagggttcttttgcttgaacgctgaggcggttaaccttgcgcctctccatcatcgccgaagcgagcgatcgtctttgactagtattctcaggcctagtcgtagcctagcatcgcctatagtgagtcgtattatcggc

SEQ ID NO: 92 - DNA, 161 bp, Artificial Sequence
  Synthetic
atgcgcaggacccggtgagccattggtcgcatggccttccttatcctgaggcggttaaccttgcgcctctccatcaacgccgaagcgagccacaacatgggtcgtgttggcgcaggcctagtcgtagcctagcatcgcctatagtgagtcgtattatcggc

SEQ ID NO: 93 - DNA, 138 bp, Artificial Sequence
  Synthetic
atgcatgtccggactgccaggtgcaaacttcacgctgaggcggttaaccttgcgcctctccatcaccgccgaagcggggcgtgaagccttattcaggtacaccgggcagggctacctatagtgagtcgtattatcggc

SEQ ID NO: 94 - DNA, 138 bp, Artificial Sequence
  Synthetic
atgcatgtccggactgccaggtgtacccattcgactgaggcggttaaccttgcgcctctccatcatcgccgaagcgagtcgttgagctaggctcaggtacaccgggcagggctacctatagtgagtcgtattatcggc

SEQ ID NO: 95 - DNA, 138 bp, Artificial Sequence
  Synthetic
atgcatgtccggactgccaggtgcagatcgcactctgaggcggttaaccttgcgcctctccatcatcgccgaagcgaagagtgcgcgagtgatcaggtacaccgggcagggctacctatagtgagtcgtattatcggc

SEQ ID NO: 96 - DNA, 138 bp, Artificial Sequence
  Synthetic
atgcatgtccggactgccaggtgcaccgtgacgactgaggcggttaaccttgcgcctctccatcatcgccgaagcgaggtcgtcggagaaatccaggtacaccgggcagggctacctatagtgagtcgtattatcggc

SEQ ID NO: 97 - DNA, 138 bp, Artificial Sequence
  Synthetic
atgcatgtccggactgccacggtgcacttatgctctgaggcggttaacattgcgcctctccatcatcgccgaagcgagagcattgacacgtgtcaggtacaccgggcagggctacctatagtgagtcgtattatcggc

SEQ ID NO: 98 - DNA, 149 bp, Artificial Sequence
  Synthetic
atgcgcaggacccggtgagccaggaacctgaacgtaccgcttctacatttataatggggcggggaaccagcgacatccatcatcgccgaagcgtggttcccaggcctagtcgtagcctagcatcgcctatagtgagtcgtattatcggc

SEQ ID NO: 99 - DNA, 161 bp, Artificial Sequence
  Synthetic
atgcgcaggacccggtgagccagctcgggaccaacactcgcaagactgaggcggttaaccttgcgcctctccatcagcgccgaagcgcgtcaagcaatctcaactaaattctcaggcctagtcgtagcctagcatcgcctatagtgagtcgtattatcggc

SEQ ID NO: 100 - DNA, 175 bp, Artificial Sequence
  Synthetic
atgcgcaggacccggtgagccagacgggtcgcttcattcttgttcgtaaatcctgaggcggttaaccttgcgcctctccatcagcgccgaagcgcggactactagcaattggtggtgcagactactcaggcctagtcgtagcctagcatcgcctatagtgagtcgtattatcggc

SEQ ID NO: 101 - DNA, 161 bp, Artificial Sequence
  Synthetic
atgcgcaggacccggtgagccaggcgtaggtgcgcgttagagcacctgaggcggttaaccttgcgcctctccatcaacgccgaagcgaggccaggctaagtgctaaacttggcaggcctagtcgtagcctagcatcgcctatagtgagtcgtattatcggc

SEQ ID NO: 102 - DNA, 161 bp, Artificial Sequence
  Synthetic
atgcgcaggacccggtgagccaggggcgcgtggctccgtgggtcgctgaggcggttaaccttgcgcctctccatcagcgccgaagcgaggatacggtaggtgccgaccatcacaggcctagtcgtagcctagcatcgcctatagtgagtcgtattatcggc

SEQ ID NO: 103 - RNA, 54 nt, Artificial Sequence
  Synthetic
ggaaccccgcuucggcggugauggagaggcgcaagguuaaccgccucagguucc

SEQ ID NO: 104 - RNA, 45 nt, Artificial Sequence
  Synthetic
  misc_feature (8)..(8): n represents a randomized region and can be a, c, g, or u; see specification as filed for detailed description of preferred embodiments
ggaaccunaugauggagaggcgcaagguuaaccgccucagguucc

SEQ ID NO: 105 - RNA, 45 nt, Artificial Sequence
  Synthetic
  misc_feature (8)..(8): n represents a randomized region and can be a, c, g, or u; see specification as filed for detailed description of preferred embodiments
ggaacccngugauggagaggcgcaagguuaaccgccucagguucc

SEQ ID NO: 106 - RNA, 45 nt, Artificial Sequence
  Synthetic
  misc_feature (7)..(9): n represents a randomized region and can be a, c, g, or u; see specification as filed for detailed description of preferred embodiments
ggaaccnnnugauggagaggcgcaagguuaaccgccucagguucc

SEQ ID NO: 107 - RNA, 74 nt, Artificial Sequence
  Synthetic
  misc_feature (8)..(37): n is a, c, g, or u
ggaaccunnnnnnnnnnnnnnnnnnnnnnnnnnnnnnaugauggagaggcgcaagguuaaccgccucagguucc

SEQ ID NO: 108 - RNA, 74 nt, Artificial Sequence
  Synthetic
  misc_feature (8)..(37): n is a, c, g, or u
ggaacccnnnnnnnnnnnnnnnnnnnnnnnnnnnnnngugauggagaggcgcaagguuaaccgccucagguucc

SEQ ID NO: 109 - RNA, 74 nt, Artificial Sequence
  Synthetic
  misc_feature (7)..(38): n is a, c, g, or u
ggaaccnnnnnnnnnnnnnnnnnnnnnnnnnnnnnnnnugauggagaggcgcaagguuaaccgccucagguucc

SEQ ID NO: 110 - RNA, 89 nt, Artificial Sequence
  Synthetic
  misc_feature (8)..(52): n is a, c, g, or u
ggaaccunnnnnnnnnnnnnnnnnnnnnnnnnnnnnnnnnnnnnnnnnnnnnaugauggagaggcgcaagguuaaccgccucagguucc

SEQ ID NO: 111 - RNA, 89 nt, Artificial Sequence
  Synthetic
  misc_feature (8)..(52): n is a, c, g, or u
ggaacccnnnnnnnnnnnnnnnnnnnnnnnnnnnnnnnnnnnnnnnnnnnnngugauggagaggcgcaagguuaaccgccucagguucc

SEQ ID NO: 112 - RNA, 89 nt, Artificial Sequence
  Synthetic
  misc_feature (7)..(53): n is a, c, g, or u
ggaaccnnnnnnnnnnnnnnnnnnnnnnnnnnnnnnnnnnnnnnnnnnnnnnnugauggagaggcgcaagguuaaccgccucagguucc

SEQ ID NO: 113 - RNA, 104 nt, Artificial Sequence
  Synthetic
  misc_feature (8)..(67): n is a, c, g, or u
ggaaccunnnnnnnnnnnnnnnnnnnnnnnnnnnnnnnnnnnnnnnnnnnnnnnnnnnnnnnnnnnnaugauggagaggcgcaagguuaaccgccucagguucc

SEQ ID NO: 114 - RNA, 104 nt, Artificial Sequence
  Synthetic
  misc_feature (8)..(67): n is a, c, g, or u
ggaacccnnnnnnnnnnnnnnnnnnnnnnnnnnnnnnnnnnnnnnnnnnnnnnnnnnnnnnnnnnnngugauggagaggcgcaagguuaaccgccucagguucc

SEQ ID NO: 115 - RNA, 104 nt, Artificial Sequence
  Synthetic
  misc_feature (7)..(68): n is a, c, g, or u
ggaaccnnnnnnnnnnnnnnnnnnnnnnnnnnnnnnnnnnnnnnnnnnnnnnnnnnnnnnnnnnnnnnugauggagaggcgcaagguuaaccgccucagguucc

SEQ ID NO: 116 - RNA, 46 nt, Artificial Sequence
  Synthetic
  misc_feature (1)..(1): n represents a randomized region and can be a, c, g, or u; see specification as filed for detailed description of preferred embodiments
  misc_feature (46)..(46): n represents a randomized region and can be a, c, g, or u; see specification as filed for detailed description of preferred embodiments
ncucgcuucggcgaugauggagaggcgcaagguuaaccgccucagn

SEQ ID NO: 117 - RNA, 46 nt, Artificial Sequence
  Synthetic
  misc_feature (1)..(1): n represents a randomized region and can be a, c, g, or u; see specification as filed for detailed description of preferred embodiments
  misc_feature (46)..(46): n represents a randomized region and can be a, c, g, or u; see specification as filed for detailed description of preferred embodiments
ncccgcuucggcggugauggagaggcgcaagguuaaccgccucagn

SEQ ID NO: 118 - RNA, 46 nt, Artificial Sequence
  Synthetic
  misc_feature (1)..(1): n represents a randomized region and can be a, c, g, or u; see specification as filed for detailed description of preferred embodiments
  misc_feature (3)..(3): n is a, c, g, or u
  misc_feature (14)..(14): n is a, c, g, or u
  misc_feature (46)..(46): n represents a randomized region and can be a, c, g, or u; see specification as filed for detailed description of preferred embodiments
ncncgcuucggcgnugauggagaggcgcaagguuaaccgccucagn

SEQ ID NO: 119 - RNA, 35 nt, Artificial Sequence
  Synthetic
  misc_feature (27)..(27): n represents a randomized region and can be a, c, g, or u; see specification as filed for detailed description of preferred embodiments
ggaaccucgcuucggcgaugauggagncagguucc

SEQ ID NO: 120 - RNA, 35 nt, Artificial Sequence
  Synthetic
  misc_feature (27)..(27): n represents a randomized region and can be a, c, g, or u; see specification as filed for detailed description of preferred embodiments
ggaaccccgcuucggcggugauggagncagguucc

SEQ ID NO: 121 - RNA, 35 nt, Artificial Sequence
  Synthetic
  misc_feature (7)..(7): n is a, c, g, or u
  misc_feature (18)..(18): n is a, c, g, or u
  misc_feature (27)..(27): n represents a randomized region and can be a, c, g, or u; see specification as filed for detailed description of preferred embodiments
ggaaccncgcuucggcgnugauggagncagguucc

SEQ ID NO: 122 - RNA, 74 nt, Artificial Sequence
  Synthetic
  misc_feature (1)..(15): n is a, c, g, or u
  misc_feature (60)..(74): n is a, c, g, or u
nnnnnnnnnnnnnnncucgcuucggcgaugauggagaggcgcaagguuaaccgccucagnnnnnnnnnnnnnnn

SEQ ID NO: 123 - RNA, 74 nt, Artificial Sequence
  Synthetic
  misc_feature (1)..(15): n is a, c, g, or u
  misc_feature (60)..(74): n is a, c, g, or u
nnnnnnnnnnnnnnncccgcuucggcggugauggagaggcgcaagguuaaccgccucagnnnnnnnnnnnnnnn

SEQ ID NO: 124 - RNA, 74 nt, Artificial Sequence
  Synthetic
  misc_feature (1)..(15): n is a, c, g, or u
  misc_feature (17)..(17): n is a, c, g, or u
  misc_feature (28)..(28): n is a, c, g, or u
  misc_feature (60)..(74): n is a, c, g, or u
nnnnnnnnnnnnnnncncgcuucggcgnugauggagaggcgcaagguuaaccgccucagnnnnnnnnnnnnnnn

SEQ ID NO: 125 - RNA, 64 nt, Artificial Sequence
  Synthetic
  misc_feature (27)..(56): n is a, c, g, or u
ggaaccucgcuucggcgaugauggagnnnnnnnnnnnnnnnnnnnnnnnnnnnnnncagguucc

SEQ ID NO: 126 - RNA, 64 nt, Artificial Sequence
  Synthetic
  misc_feature (27)..(56): n is a, c, g, or u
ggaaccccgcuucggcggugauggagnnnnnnnnnnnnnnnnnnnnnnnnnnnnnncagguucc

SEQ ID NO: 127 - RNA, 64 nt, Artificial Sequence
  Synthetic
  misc_feature (7)..(7): n is a, c, g, or u
  misc_feature (18)..(18): n is a, c, g, or u
  misc_feature (27)..(56): n is a, c, g, or u
ggaaccncgcuucggcgnugauggagnnnnnnnnnnnnnnnnnnnnnnnnnnnnnncagguucc

SEQ ID NO: 128 - RNA, 90 nt, Artificial Sequence
  Synthetic
  misc_feature (1)..(23): n is a, c, g, or u
  misc_feature (68)..(90): n is a, c, g, or u
nnnnnnnnnnnnnnnnnnnnnnncucgcuucggcgaugauggagaggcgcaagguuaaccgccucagnnnnnnnnnnnnnnnnnnnnnnn

SEQ ID NO: 129 - RNA, 90 nt, Artificial Sequence
  Synthetic
  misc_feature (1)..(23): n is a, c, g, or u
  misc_feature (68)..(90): n is a, c, g, or u
nnnnnnnnnnnnnnnnnnnnnnncccgcuucggcggugauggagaggcgcaagguuaaccgccucagnnnnnnnnnnnnnnnnnnnnnnn

SEQ ID NO: 130 - RNA, 90 nt, Artificial Sequence
  Synthetic
  misc_feature (1)..(23): n is a, c, g, or u
  misc_feature (25)..(25): n is a, c, g, or u
  misc_feature (36)..(36): n is a, c, g, or u
  misc_feature (68)..(90): n is a, c, g, or u
nnnnnnnnnnnnnnnnnnnnnnncncgcuucggcgnugauggagaggcgcaagguuaaccgccucagnnnnnnnnnnnnnnnnnnnnnnn

SEQ ID NO: 131 - RNA, 79 nt, Artificial Sequence
  Synthetic
  misc_feature (27)..(71): n is a, c, g, or u
ggaaccucgcuucggcgaugauggagnnnnnnnnnnnnnnnnnnnnnnnnnnnnnnnnnnnnnnnnnnnnncagguucc

SEQ ID NO: 132 - RNA, 79 nt, Artificial Sequence
  Synthetic
  misc_feature (27)..(71): n is a, c, g, or u
ggaaccccgcuucggcggugauggagnnnnnnnnnnnnnnnnnnnnnnnnnnnnnnnnnnnnnnnnnnnnncagguucc

SEQ ID NO: 133 - RNA, 79 nt, Artificial Sequence
  Synthetic
  misc_feature (7)..(7): n is a, c, g, or u
  misc_feature (18)..(18): n is a, c, g, or u
  misc_feature (27)..(71): n is a, c, g, or u
ggaaccncgcuucggcgnugauggagnnnnnnnnnnnnnnnnnnnnnnnnnnnnnnnnnnnnnnnnnnnnncagguucc

SEQ ID NO: 134 - RNA, 104 nt, Artificial Sequence
  Synthetic
  misc_feature (1)..(30): n is a, c, g, or u
  misc_feature (75)..(104): n is a, c, g, or u
nnnnnnnnnnnnnnnnnnnnnnnnnnnnnncucgcuucggcgaugauggagaggcgcaagguuaaccgccucagnnnnnnnnnnnnnnnnnnnnnnnnnnnnnn

SEQ ID NO: 135 - RNA, 104 nt, Artificial Sequence
  Synthetic
  misc_feature (1)..(30): n is a, c, g, or u
  misc_feature (75)..(104): n is a, c, g, or u
nnnnnnnnnnnnnnnnnnnnnnnnnnnnnncccgcuucggcggugauggagaggcgcaagguuaaccgccucagnnnnnnnnnnnnnnnnnnnnnnnnnnnnnn

SEQ ID NO: 136 - RNA, 104 nt, Artificial Sequence
  Synthetic
  misc_feature (1)..(30): n is a, c, g, or u
  misc_feature (32)..(32): n is a, c, g, or u
  misc_feature (43)..(43): n is a, c, g, or u
  misc_feature (75)..(104): n is a, c, g, or u
nnnnnnnnnnnnnnnnnnnnnnnnnnnnnncncgcuucggcgnugauggagaggcgcaagguuaaccgccucagnnnnnnnnnnnnnnnnnnnnnnnnnnnnnn

SEQ ID NO: 137 - RNA, 94 nt, Artificial Sequence
  Synthetic
  misc_feature (27)..(86): n is a, c, g, or u
ggaaccucgcuucggcgaugauggagnnnnnnnnnnnnnnnnnnnnnnnnnnnnnnnnnnnnnnnnnnnnnnnnnnnnnnnnnnnncagguucc

SEQ ID NO: 138 - RNA, 94 nt, Artificial Sequence
  Synthetic
  misc_feature (27)..(86): n is a, c, g, or u
ggaaccccgcuucggcggugauggagnnnnnnnnnnnnnnnnnnnnnnnnnnnnnnnnnnnnnnnnnnnnnnnnnnnnnnnnnnnncagguucc

SEQ ID NO: 139 - RNA, 94 nt, Artificial Sequence
  Synthetic
  misc_feature (7)..(7): n is a, c, g, or u
  misc_feature (18)..(18): n is a, c, g, or u
  misc_feature (27)..(86): n is a, c, g, or u
ggaaccncgcuucggcgnugauggagnnnnnnnnnnnnnnnnnnnnnnnnnnnnnnnnnnnnnnnnnnnnnnnnnnnnnnnnnnnncagguucc

SEQ ID NO: 140 - RNA, 138 nt, Artificial Sequence
  Synthetic
  misc_feature (53)..(82): n is a, c, g, or u
gccgauaauacgacucacuauagguagcccugcccgguguaccugggaaccunnnnnnnnnnnnnnnnnnnnnnnnnnnnnnaugauggagaggcgcaagguuaaccgccucagguuccuggcaguccggacaugcau

SEQ ID NO: 141 - RNA, 138 nt, Artificial Sequence
  Synthetic
  misc_feature (53)..(82): n is a, c, g, or u
gccgauaauacgacucacuauagguagcccugcccgguguaccugggaacccnnnnnnnnnnnnnnnnnnnnnnnnnnnnnngugauggagaggcgcaagguuaaccgccucagguuccuggcaguccggacaugcau

SEQ ID NO: 142 - RNA, 138 nt, Artificial Sequence
  Synthetic
  misc_feature (52)..(83): n is a, c, g, or u
gccgauaauacgacucacuauagguagcccugcccgguguaccugggaaccnnnnnnnnnnnnnnnnnnnnnnnnnnnnnnnnugauggagaggcgcaagguuaaccgccucagguuccuggcaguccggacaugcau

SEQ ID NO: 143 - RNA, 153 nt, Artificial Sequence
  Synthetic
  misc_feature (53)..(97): n is a, c, g, or u
gccgauaauacgacucacuauagguagcccugcccgguguaccugggaaccunnnnnnnnnnnnnnnnnnnnnnnnnnnnnnnnnnnnnnnnnnnnnaugauggagaggcgcaagguuaaccgccucagguuccuggcaguccggacaugcau

SEQ ID NO: 144 - RNA, 153 nt, Artificial Sequence
  Synthetic
  misc_feature (53)..(97): n is a, c, g, or u
gccgauaauacgacucacuauagguagcccugcccgguguaccugggaacccnnnnnnnnnnnnnnnnnnnnnnnnnnnnnnnnnnnnnnnnnnnnngugauggagaggcgcaagguuaaccgccucagguuccuggcaguccggacaugcau

SEQ ID NO: 145 - RNA, 153 nt, Artificial Sequence
  Synthetic
  misc_feature (52)..(98): n is a, c, g, or u
gccgauaauacgacucacuauagguagcccugcccgguguaccugggaaccnnnnnnnnnnnnnnnnnnnnnnnnnnnnnnnnnnnnnnnnnnnnnnnugauggagaggcgcaagguuaaccgccucagguuccuggcaguccggacaugcau

SEQ ID NO: 146 - RNA, 168 nt, Artificial Sequence
  Synthetic
  misc_feature (53)..(112): n is a, c, g, or u
gccgauaauacgacucacuauagguagcccugcccgguguaccugggaaccunnnnnnnnnnnnnnnnnnnnnnnnnnnnnnnnnnnnnnnnnnnnnnnnnnnnnnnnnnnnaugauggagaggcgcaagguuaaccgccucagguuccuggcaguccggacaugcau

SEQ ID NO: 147 - RNA, 168 nt, Artificial Sequence
  Synthetic
  misc_feature (53)..(112): n is a, c, g, or u
gccgauaauacgacucacuauagguagcccugcccgguguaccugggaacccnnnnnnnnnnnnnnnnnnnnnnnnnnnnnnnnnnnnnnnnnnnnnnnnnnnnnnnnnnnngugauggagaggcgcaagguuaaccgccucagguuccuggcaguccggacaugcau

SEQ ID NO: 148 - RNA, 168 nt, Artificial Sequence
  Synthetic
  misc_feature (52)..(113): n is a, c, g, or u
gccgauaauacgacucacuauagguagcccugcccgguguaccugggaaccnnnnnnnnnnnnnnnnnnnnnnnnnnnnnnnnnnnnnnnnnnnnnnnnnnnnnnnnnnnnnnugauggagaggcgcaagguuaaccgccucagguuccuggcaguccggacaugcau

SEQ ID NO: 149 - RNA, 138 nt, Artificial Sequence
  Synthetic
  misc_feature (46)..(60): n is a, c, g, or u
  misc_feature (105)..(119): n is a, c, g, or u
gccgauaauacgacucacuauagguagcccugcccgguguaccugnnnnnnnnnnnnnnncucgcuucggcgaugauggagaggcgcaagguuaaccgccucagnnnnnnnnnnnnnnnuggcaguccggacaugcau

SEQ ID NO: 150 - RNA, 139 nt, Artificial Sequence
  Synthetic
  misc_feature (46)..(60): n is a, c, g, or u
  misc_feature (105)..(119): n is a, c, g, or u
gccgauaauacgacucacuauagguagcccugcccgguguaccugnnnnnnnnnnnnnnncccgcuucggcggugauggagaggcgcaagguuaaccgccucagnnnnnnnnnnnnnnnuuggcaguccggacaugcau

SEQ ID NO: 151 - RNA, 139 nt, Artificial Sequence
  Synthetic
  misc_feature (46)..(60): n is a, c, g, or u
  misc_feature (62)..(62): n is a, c, g, or u
  misc_feature (73)..(73): n is a, c, g, or u
  misc_feature (105)..(119): n is a, c, g, or u
gccgauaauacgacucacuauagguagcccugcccgguguaccugnnnnnnnnnnnnnnncncgcuucggcgnugauggagaggcgcaagguuaaccgccucagnnnnnnnnnnnnnnnuuggcaguccggacaugcau

SEQ ID NO: 152 - RNA, 128 nt, Artificial Sequence
  Synthetic
  misc_feature (72)..(101): n is a, c, g, or u
gccgauaauacgacucacuauagguagcccugcccgguguaccugggaaccucgcuucggcgaugauggagnnnnnnnnnnnnnnnnnnnnnnnnnnnnnncagguuccuggcaguccggacaugcau

SEQ ID NO: 153 - RNA, 128 nt, Artificial Sequence
  Synthetic
  misc_feature (72)..(101): n is a, c, g, or u
gccgauaauacgacucacuauagguagcccugcccgguguaccugggaaccccgcuucggcggugauggagnnnnnnnnnnnnnnnnnnnnnnnnnnnnnncagguuccuggcaguccggacaugcau

SEQ ID NO: 154 - RNA, 128 nt, Artificial Sequence
  Synthetic
  misc_feature (52)..(52): n is a, c, g, or u
  misc_feature (63)..(63): n is a, c, g, or u
  misc_feature (72)..(101): n is a, c, g, or u
gccgauaauacgacucacuauagguagcccugcccgguguaccugggaaccncgcuucggcgnugauggagnnnnnnnnnnnnnnnnnnnnnnnnnnnnnncagguuccuggcaguccggacaugcau

SEQ ID NO: 155 - RNA, 161 nt, Artificial Sequence
  Synthetic
  misc_feature (50)..(72): n is a, c, g, or u
  misc_feature (74)..(74): n is a, c, g, or u
  misc_feature (85)..(85): n is a, c, g, or u
  misc_feature (117)..(139): n is a, c, g, or u
gccgauaauacgacucacuauaggcgaugcuaggcuacgacuaggccugnnnnnnnnnnnnnnnnnnnnnnncncgcuucggcgnugauggagaggcgcaagguuaaccgccucagnnnnnnnnnnnnnnnnnnnnnnnuggcucaccggguccugcgcau

SEQ ID NO: 156 - RNA, 149 nt, Artificial Sequence
  Synthetic
  misc_feature (56)..(56): n is a, c, g, or u
  misc_feature (67)..(67): n is a, c, g, or u
  misc_feature (75)..(119): n is a, c, g, or u
gccgauaauacgacucacuauaggcgaugcuaggcuacgacuaggccugggaaccncgcuucggcgnugauggannnnnnnnnnnnnnnnnnnnnnnnnnnnnnnnnnnnnnnnnnnnncagguuccuggcucaccggguccugcgcau

SEQ ID NO: 157 - RNA, 175 nt, Artificial Sequence
  Synthetic
  misc_feature (50)..(79): n is a, c, g, or u
  misc_feature (81)..(81): n is a, c, g, or u
  misc_feature (92)..(92): n is a, c, g, or u
  misc_feature (124)..(153): n is a, c, g, or u
gccgauaauacgacucacuauaggcgaugcuaggcuacgacuaggccugnnnnnnnnnnnnnnnnnnnnnnnnnnnnnncncgcuucggcgnugauggagaggcgcaagguuaaccgccucagnnnnnnnnnnnnnnnnnnnnnnnnnnnnnnuggcucaccggguccugcgcau

SEQ ID NO: 158 - RNA, 164 nt, Artificial Sequence
  Synthetic
  misc_feature (56)..(56): n is a, c, g, or u
  misc_feature (67)..(67): n is a, c, g, or u
  misc_feature (75)..(134): n is a, c, g, or u
gccgauaauacgacucacuauaggcgaugcuaggcuacgacuaggccugggaaccncgcuucggcgnugauggannnnnnnnnnnnnnnnnnnnnnnnnnnnnnnnnnnnnnnnnnnnnnnnnnnnnnnnnnnncagguuccuggcucaccggguccugcgcau

SEQ ID NO: 159 - DNA, 100 bp, Artificial Sequence
  Synthetic
ggcaacgatcgcgtctcctctccgcaccttcgaataagtacaagttgtacgcatcttttcatcccttccgcgatcgttgcctatagtgagtcgtattatc

SEQ ID NO: 160 - DNA, 15 bp, Artificial Sequence
  Synthetic
  misc_feature (1)..(15): n is a, c, g, or t
nnnnnnnnnnnnnnn

SEQ ID NO: 161 - DNA, 23 bp, Artificial Sequence
  Synthetic
  misc_feature (1)..(23): n is a, c, g, or t
nnnnnnnnnnnnnnnnnnnnnnn

SEQ ID NO: 162 - DNA, 30 bp, Artificial Sequence
  Synthetic
  misc_feature (1)..(30): n is a, c, g, or t
nnnnnnnnnnnnnnnnnnnnnnnnnnnnnn

SEQ ID NO: 163 - DNA, 45 bp, Artificial Sequence
  Synthetic
  misc_feature (1)..(45): n is a, c, g, or t
nnnnnnnnnnnnnnnnnnnnnnnnnnnnnnnnnnnnnnnnnnnnn

SEQ ID NO: 164 - DNA, 46 bp, Artificial Sequence
  Synthetic
  misc_feature (1)..(46): n is a, c, g, or t
nnnnnnnnnnnnnnnnnnnnnnnnnnnnnnnnnnnnnnnnnnnnnn

SEQ ID NO: 165 - DNA, 60 bp, Artificial Sequence
  Synthetic
  misc_feature (1)..(60): n is a, c, g, or t
nnnnnnnnnnnnnnnnnnnnnnnnnnnnnnnnnnnnnnnnnnnnnnnnnnnnnnnnnnnn

SEQ ID NO: 166 - RNA, 14 nt, Artificial Sequence
  Synthetic
  misc_feature: OS1 at 5' end, 0S2 at 3' end
  misc_feature (11)..(12): site of inside stem (IS)
ccgugauggagcag